(19) United States
(12) Patent Application Publication
(10) Pub. No.: US 2015/0026840 A1
(43) Pub. Date: Jan. 22, 2015

(54) CONSTRUCTS AND SYSTEMS AND METHODS FOR PRODUCING MICROCOMPARTMENTS

(71) Applicant: The Regents of the University of California, Oakland, CA (US)

(72) Inventors: Cheryl A. Kerfeld, Walnut Creek, CA (US); Jonathan K. Lassila, San Francisco, CA (US); James N. Kinney, Clayton, CA (US)

(73) Assignee: The Regents of the University of California, Oakland, CA (US)

(21) Appl. No.: 14/214,172

(22) Filed: Mar. 14, 2014

Related U.S. Application Data
(60) Provisional application No. 61/800,118, filed on Mar. 15, 2013.

(51) Int. Cl. C12N 15/82; C12N 15/70; C12N 15/81; C12N 15/75

(52) U.S. Cl. CPC C12N 15/8261; C12N 15/75; C12N 15/70; C12N 15/81
USPC 800/278; 435/471; 435/320.1; 435/252.33; 435/252.31; 435/254.21; 435/257.2; 435/419; 800/298; 800/317.3; 435/317.1; 435/468

(57) ABSTRACT

To produce a bacterial microcompartment shell, or a designed shell based on naturally occurring bacterial microcompartment shells in a new host organism, a synthetic operon is constructed that contains the desired shell protein genes and translation efficiency is controlled by host specific ribosomal binding sites. Proteins or other molecules can be encapsulated in the microcompartment shells by various methods described herein. The constructs can also be used to express self-assembling sheets comprised of shell proteins.

### STATEMENT OF GOVERNMENTAL SUPPORT

This invention was made with government support under Contract No. DE-AC02-05CH11231 awarded by the U.S. Department of Energy, under Contract No. DE-0000200 awarded by the Department of Energy ARPA-E, and under Grant Nos. MCB0851094 and MCB1160614 awarded by the National Science Foundation. The government has certain rights in the invention.

### CROSS-REFERENCE TO RELATED APPLICATIONS

This application is a nonprovisional of and claims priority to U.S. Provisional Patent Application No. 61/800,118, filed on Mar. 15, 2013, which is hereby incorporated by reference in its entirety for all purposes.

This application is related to co-pending nonprovisional application U.S. patent application Ser. No. 13/564,676, filed on Aug. 1, 2012, hereby incorporated by reference in its entirety. This application is related to and incorporates by reference U.S. patent application Ser. No. 13/367,260, filed on Feb. 6, 2012 in its entirety for all purposes.

### REFERENCE TO SEQUENCE LISTING AND TABLES

This application also incorporates by reference the attached sequence listings which is also found in computer-readable form in a *.txt file entitled, “IB3335US_seqlisting_ST25.txt”, created on Mar. 14, 2014.

### BACKGROUND OF THE INVENTION

1. Field of the Invention

The present invention relates to synthetic biology, especially using operons and synthetic constructs to produce microcompartments and bacterial microcompartment shells and to integrate molecules and proteins into these microcompartments, or on the microcompartment surface.

2. Related Art

Bacterial microcompartments (BMCs) encapsulate enzymes and metabolic pathways. The most well known type of BMC is the carboxysome, which fixes CO₂ in cyanobacteria. Several other types of BMC gene clusters have been identified in prokaryotes, including the propanediol utilization and ethanolamine utilization microcompartment gene clusters.

The shells of BMCs are composed of multiple paralogs of proteins containing BMC domains pfam00936 and pfam03319. Three types of shell proteins have been identified: single pfam00936 domains (“hexamer”), fusion proteins composed of two pfam00936 domains (“tandem domain”), and single pfam03319 domains (“pentamer”). Hexamer and tandem domain proteins are the major components of known microcompartment shells, while pentamer proteins are minor components. Natural BMC gene clusters vary widely in composition and gene arrangement and are defined by genes that encode shell proteins. Three types of BMC shell proteins exist, identified here as hexamers or BMC-H, tandem domains or BMC-T, and pentamers or BMC-P, that together form polyhedral shells (FIG. 1). BMC-H polypeptides contain a single domain of the pfam00936 family from the pfam database (Punta, M., Coggill, P. C., Eberhardt, R. Y., Mistry, J. & Tate, J. e. a. (2012). The Pfam protein families database. Nucleic Acids Research 40, D290-D301), about 90 amino acids, that assembles into a six-fold symmetric hexamer in crystal structures. This type of subunit represents the most abundant component of characterized BMC shells. Tandem domains (BMC-T) contain two pfam00936 domains in a single polypeptide. These proteins form trimers with a pseudo-hexameric configuration that are sometimes found stacked into a double layer in crystal structures. (Klein, M. G., Zwart, P., Bagby, S. C., Cai, F., Chisholm, S. W., Heinhorst, S., Cannon, G. C. & Kerfeld, C. A. (2009). Identification and structural analysis of a novel carboxysome shell protein with implications for metabolite transport. Journal of Molecular Biology 392, 319-333; Cai, F., Sutter, M., Cameron, J. C., Stanley, D. N., Kinney, J. N. & Kerfeld, C. A. (2013). The structure of CcmP, a tandem bacterial microcompartment domain protein from the β-carboxysome forms a subcompartment within a microcompartment. Journal of Biological Chemistry 288, 16055-16063). The third type, referred to here as pentamers or BMC-P, contain a single domain of the pfam03319 family. The five-fold symmetric assemblies formed by these proteins are presumed to occupy the vertices of icosahedral shells. Accordingly, they are a minor component of characterized BMC shells; only 60 copies of the gene product (12 pentamers) are required to close an icosahedral shell.

Previously others have expressed only naturally existing microcompartment operons or partial operons in heterologous hosts. See Bonacci W, Teng P K, Afonso B, Niederholtmeyer H, Grob P, Silver P A, Savage D F, Modularity of a carbon-fixing protein organelle, Proc. Natl. Acad. Sci. USA 2012 Jan. 10; 109(2):478-83.Epub 2011 Dec. 19. However, a general approach for producing synthetic microcompartment shell operons, synthetic microcompartment shells and integrating molecules into microcompartments has not been described.

### BRIEF SUMMARY OF THE INVENTION

The present invention provides for constructs and systems and methods for producing microcompartments and microcompartment shells or protein scaffolds based on microcompartment architecture.

In one embodiment, the present invention provides for a construct or an expression cassette comprising a polynucleotide encoding a cluster of microcompartment genes, wherein the cluster comprises a set of microcompartment genes necessary for the expression of a microcompartment shell in a host cell, and wherein at least one of the genes is preceded by a ribosomal binding site to control expression in said host cell.

The expression cassette can be used to provide a cell comprising in its genome at least one stably incorporated expression cassette, where the expression cassette comprising a heterologous nucleotide sequence or a fragment thereof operably linked to a promoter that drives expression in the cell and operably linked to a ribosomal binding site that controls expression efficiency in the cell.

The present invention further describes methods for production of BMCs in bacterial hosts such as Escherichia coli and other host organisms. In one embodiment, a method comprising producing a synthetic operon for expression of shell protein genes in a host organism with a specific ordering of the genes and a specific set of ribosomal binding site (RBS) sequences to produce the shell proteins in desired ratios. In some embodiments, the method further comprises coexpression with peptide tag sequences for incorporation of proteins into the microcompartment shells. In other embodiments, the method further comprises coexpression of microcompartments with proteins without peptide tag sequences for incorporation of proteins into the microcompartment shells.

Also provided are methods for enhancing metabolic activity in an organism. In one method, comprising introducing into an organism at least one expression cassette operably linked to a promoter that drives expression in the organism, where the expression cassette comprising a cluster of microcompartment genes identified from a bacterial species, wherein the cluster comprising a set microcompartment genes necessary for the expression of a microcompartment that has metabolic activity, wherein the microcompartment genes further comprise a polynucleotide or a fragment thereof which acts as a ribosomal binding site that controls expression efficiency in the organism.

In various embodiments, a common motif (peptide) found in a subset of proteins presumed to be encapsulated in functionally diverse bacterial microcompartments (BMCs), and adjacent linker regions can also be included in the construct for targeting proteins to BMCs. All BMC targeting peptides share general properties such as a region predicted to have an alpha helical conformation, adjacent to poorly conserved segment(s) of primary structure enriched in proline and glycine; for each type of encapsulated protein, for each functionally distinct BMC. Amino acid properties are conserved in many of the positions within these peptides. In some embodiments, previously identified consensus targeting peptides specific to various BMC types can be used with the present constructs, systems and methods.

In one embodiment, shell proteins are described and methods for assembling Haliangium ochraceum shell proteins into a synthetic operon, producing the H. ochraceum microcompartment shells in Escherichia coli as the host organism, and incorporating proteins into the microcompartment shells using H. ochraceum peptide tag sequences are described.

### BRIEF DESCRIPTION OF THE SEQUENCES

SEQ ID NO:1 is a high ribosomal binding site (RBS) sequence from E. coli.

SEQ ID NO:2 is a medium RBS sequence that promotes medium translation efficiency in E. coli.

SEQ ID NO:3 is a low RBS sequence that promotes low translation efficiency in E. coli.

SEQ ID NO:4 is the natural RBS site from the H. neapolitanus shell protein CsoS1C.

SEQ ID NO:5 is a DNA sequence that encodes a polypeptide of Verminephrobacter eiseniae EF01-2.

SEQ ID NO:6 is a BMC gene product identified from Haliangium ochraceum SMP-2, DSM 14365.

SEQ ID NO:7 is a DNA sequence selected to encode the protein sequence of SEQ ID NO:6 while utilizing high-frequency codons from E. coli, the host organism.

SEQ ID NO:8 is a BMC gene product identified from Haliangium ochraceum SMP-2, DSM 14365.

SEQ ID NO:9 is a DNA sequence selected to encode the protein sequence of SEQ ID NO:8 while utilizing high-frequency codons from E. coli, the host organism.

SEQ ID NO:10 is a BMC gene product identified from Haliangium ochraceum SMP-2, DSM 14365.

SEQ ID NO:11 is a DNA sequence selected to encode the protein sequence of SEQ ID NO:10 while utilizing high-frequency codons from E. coli, the host organism.

SEQ ID NO:12 is a BMC gene product identified from Haliangium ochraceum SMP-2, DSM 14365.

SEQ ID NO:13 is a DNA sequence selected to encode the protein sequence of SEQ ID NO:12 while utilizing high-frequency codons from E. coli, the host organism.

SEQ ID NO:14 is a BMC gene product identified from Haliangium ochraceum SMP-2, DSM 14365.

SEQ ID NO:15 is a DNA sequence selected to encode the protein sequence of SEQ ID NO:14 while utilizing high-frequency codons from E. coli, the host organism.

SEQ ID NO:16 is a BMC gene product identified from Haliangium ochraceum SMP-2, DSM 14365.

SEQ ID NO:17 is a DNA sequence selected to encode the protein sequence of SEQ ID NO:16 while utilizing high-frequency codons from E. coli, the host organism.

SEQ ID NO:18 is a BMC gene product identified from Haliangium ochraceum SMP-2, DSM 14365.

SEQ ID NO:19 is a DNA sequence selected to encode the protein sequence of SEQ ID NO:18 while utilizing high-frequency codons from E. coli, the host organism.

SEQ ID NO:20 is a RBS sequence.

SEQ ID NO:21 is a RBS sequence.

SEQ ID NO:22 is a RBS sequence.

SEQ ID NO:23 is a protein sequence that was fused to the N-terminus of GFP in a Tag-GFP construct.

SEQ ID NO:24 is a DNA sequence used with Tag-GFP construct of SEQ ID: 23 derived from the N-terminus of an aldehyde dehydrogenase from H. ochraceum.

SEQ ID NO:25 is a protein sequence that was fused to the N-terminus of GFP of a full Enzyme-GFP construct with protein derived from aldehyde dehydrogenase (Protein accession number YP_{—}003270182; Locus tag: Hoch_{—}5813).

SEQ ID NO:26 is DNA sequence used to encode the fused protein of SEQ ID NO:25.

SEQ ID NO:27 is a protein sequence fused to C-terminus of GFP of a noncognate Tag-GFP construct.

SEQ ID NO:28 is a DNA sequence fused to the 3′ end of the DNA encoding GFP of a noncognate Tag-GFP construct.

SEQ ID NO:29 is a RBS sequence for operon of Example 4.

SEQ ID NO:30 is a protein sequence of YP_{—}884687.

SEQ ID NO:31 is a DNA sequence of YP_{—}884687.

SEQ ID NO:32 is a RBS sequence for operon of Example 4.

SEQ ID NO:33 is a protein sequence of YP_{—}884690.

SEQ ID NO:34 is a DNA sequence of YP_{—}884690.

SEQ ID NO:35 is a RBS sequence for operon of Example 4.

SEQ ID NO:36 is a protein sequence of YP_{—}884688.

SEQ ID NO:37 is a protein sequence of YP_{—}884688.

SEQ ID NO:38 is a non-native enzyme Rubisco protein sequence.

SEQ ID NO:39 is a non-native enzyme Rubisco DNA sequence.

SEQ ID NO:40 is the hexamer protein sequence for synthetic operon of Example 6 from Thermosynechococcus elongatus BP-1: NC_{—}004113.

SEQ ID NO:41 is the hexamer DNA sequence for synthetic operon of Example 6 from Thermosynechococcus elongatus BP-1: NC_{—}004113. This sequence has been codon-optimized for expression in E. coli.

SEQ ID NO:42 is the tandem domain protein sequence for synthetic operon of Example 6 from Thermosynechococcus elongatus BP-1: NC_{—}004113.

SEQ ID NO:43 is the tandem domain DNA sequence for synthetic operon of Example 6 from Thermosynechococcus elongatus BP-1: NC_{—}004113. This sequence has been codon-optimized for expression in E. coli.

SEQ ID NO:44 is the pentamer protein sequence for synthetic operon of Example 6 from Thermosynechococcus elongatus BP-1: NC_{—}004113.

SEQ ID NO:45 is the pentamer DNA sequence for synthetic operon of Example 6 from Thermosynechococcus elongatus BP-1: NC_{—}004113. This sequence has been codon-optimized for expression in E. coli.

SEQ ID NO:46 is the B1010 ribosome binding site used in the expression constructs in example 6.

SEQ ID NO:47 is the B1001 ribosome binding site used in the expression constructs in example 6.

SEQ ID NO:48 is the protein sequence of the SuperFolderGFP used in the SFGFP-CcmK2 fusion proteins.

SEQ ID NO:49 is the DNA sequence of the SuperFolderGFP. This sequence does not contain a stop codon.

SEQ ID NO:50 is the protein sequence for the Haliangium ochraceum targeting peptide found on the N-terminus of the aldolase gene encoded in the operon (Hoch_{—}4427) with RDDLVRVIREELVRAL (SEQ ID NO: 51) corresponding to the predicted alpha helix/EP based on the criteria of Kinney et al., 2011. SEQ ID NO: 52 is the DNA sequence which encodes the targeting peptide optimized for E. coli.

SEQ ID NO:53 is the DNA sequence for the Haliangium ochraceum targeting peptide found on the N-terminus of the aldehyde dehydrogenase gene encoded in the operon (Hoch_{—}4427) codon optimized for E. coli. SEQ ID NO: 54 is the targeting peptide sequence found on the N-terminus of the aldehyde dehydrogenase gene: ALREDRIAEIVERVLARL.

### BRIEF DESCRIPTION OF THE DRAWINGS AND TABLES

FIG. 1A is diagram of synthetic operon construction. Shell proteins are placed under control of ribosomal binding sites (RBS) of varying translation start efficiency depending on their expected roles in BMC shell assembly. FIG. 1B shows three types of shell proteins that assemble to form icosahedral BMC shells. One polypeptide chain is indicated in color in example crystal structures for each type.

FIG. 2. Diagram of the construct used to express H. ochraceum shell proteins in E. coli. Above each gene are listed the protein accession numbers for the protein sequences encoded by the genes.

FIG. 3 is an electron microscopy image of microcompartment shells isolated from E. coli cell extract.

FIG. 4 shows a Western blot with anti-GFP antibody of microcompartment shells expressed with four types of constructs: GFP alone, GFP attached to an encapsulation tag from a different organism, GFP attached to a tag from Haliangium ochraceum, and GFP attached to the aldehyde dehydrogenase enzyme associated with the Haliangium ochraceum microcompartment. When encapsulation tags were included, GFP was detected in the microcompartment fraction.

FIG. 5 illustrates the construction of a synthetic operon for expression of shell protein genes from Mycobacterium smegmatis in E. coli host cells.

FIG. 6 shows negatively-stained electron microscope image of shells produced from expression of M. smegmatis shell proteins in E. coli.

FIG. 7 shows the capture of the non-native enzyme Rubisco by the microcompartment shells produced by H. ochraceum. Left is SDS-PAGE gel. Right is a Western blot with anti-Rubisco.

FIG. 8 shows (A) Ultracentrifuge pull-down of shell proteins upon expression of H. ochraceum BMC shell protein construct in E. coli. The shell proteins copurified with sucrose gradient ultracentrifugation or agarose gel electrophoresis. Locus tags are indicated for proteins identified by mass spectrometry. (B) The synthetic operon for expression of the seven H. ochraceum shell proteins with locus tags indicated.

FIG. 9 shows (A) Negatively stained electron microscopy image of H. ochraceum microcompartment shells purified from E. coli. White bar indicates 50 nm. (B) and (C) Negatively stained electron microscopy image of shells incubated with 5 nm gold particles conjugated to anti-rabbit and polyclonal antibodies raised against H. ochraceum BMC-T(5812). Black bar indicates 50 nm. The distances from shells to gold particles is consistent with the ˜7 nm expected distance given the antibody dimensions.

FIG. 10 shows testing requirements for shell formation. (A) TEM images of pure BMC-H protein show two-dimensional assemblies of packed hexagons. The white bar indicates 50 nm. (B) SDS-PAGE of purified compartments formed upon shell protein expression with or without the BMC-P proteins. (C) TEM images of purified compartments formed upon expression with or without BMC-P. Black bars indicate 20 nm. (D) SDS-PAGE of ultracentrifuge extracts from cells expressing the full H. ochraceum construct and mutants. Stop codons were inserted early in each of the three BMC-T genes indicated, labeled by their locus tag numbers. Preparations were done in parallel and identical volumes of sample were loaded.

FIG. 11 shows (A) Models for shell protein stoichiometry. Four models are considered, with different stoichiometry of shell proteins in each hexagonal unit. In these models, all BMC-T units are treated identically. The number of each type of unit per icosahedral face is indicated in parentheses and possible layouts of the icosahedral face are shown. (B) Model 5 allows for different stoichiometries and structural roles for the different BMC-T units. The model is speculative, but supported by experimental data as described in the text. (C) The structural model for the icosahedron, with 13 hexagonal units per face, is well defined by the dimensions of the shells. The model is colored according to the structural roles suggested in Model 5, with BMC-H in blue, BMC-T in red and pink, and BMC-P in green, as in FIG. 1B.

FIG. 12 shows Western blot with anti-GFP antibody of purified shell fractions following co-expression of shell proteins with three constructs. SDS-PAGE for the Hoch_{—}5812 BMC-T unit is shown to indicate that equivalent quantities of shell protein were loaded.

FIG. 13 shows A) TEM image of synthetic microcompartments (shells) composed of the proteins CcmO(BMC-T), CcmK2(BMC-H), CcmL(BMC-P), and a SFGFP(ns)CcmK2(BMC-H) fusion from Thermosynechococcus elongatus stained with 1% ammonium molybdate. 30,000× Mag. Scale bar=20 nm. B) TEM of synthetic Thermosynechococcus elongatus CcmO(BMC-T)-CcmK2(BMC-H)-CcmL(BMC-P)-SFGFP(ns)CcmK2(BMC-H) shells stained with 1% uranyl acetate. 15,000× Mag.). Scale bar=50 nm. “ns” indicates no stop in reading frame between superfolder gfp and ccmk2 genes to produce a SFGFP-ccmK2(BMC-H) fusion protein. C) Diagram of shell encoding synthetic operon construction. Shell proteins, identified by NCBI Accession numbers, are placed in control of ribosome binding sites (RBS) of various translation start efficiency, their relative strengths indicated as either Med (medium) or High, within a pET-23b expression vector. ADW83736_NP_{—}681737_fusion identifies the SuperFolderGFP(SFGFP)-CcmK2(BMC-H) fusion protein. B1010 corresponds to ribosome binding site (SEQ ID NO:46) and B1010 corresponds to ribosome binding site (SEQ ID NO:47).

FIG. 14: A) TEM image of synthetic shells composed of the proteins CcmO(BMC-T), CcmK2(BMC-H), and CcmL(BMC-P) from Thermosynechococcus elongatus stained with 1% uranyl acetate. 30,000× Mag. Scale bar=50 nm. B) Diagram of shell encoding synthetic operon construction. Shell proteins, identified by NCBI Accession numbers, are placed in control of ribosome binding sites (RBS) of various translation start efficiency, their relative strengths indicated as either Med (medium) or High, within a pET-23b expression vector. B1010 corresponds to ribosome binding site (SEQ ID NO:46) and B1001 corresponds to ribosome binding site (SEQ ID NO:47).

FIG. 15 shows A) TEM image of synthetic shells composed of the proteins CcmO(BMC-T), CcmK2(BMC-H), and CcmL(BMC-P) from Thermosynechococcus elongatus stained with 1% uranyl acetate. 18,500× Mag. Scale bar=100 nm. Shells shown were precipitated out of solution at an ammonium sulfate concentration of 5%. Not shown is an SDS-PAGE gel of ammonium sulfate precipitation purified shells. Percent ammonium sulfate in sample is indicated at the top of each lane. Bands likely corresponding CcmK2(BMC-H), CcmO(BMC-T), CcmL(BMC-P) shell proteins and molecular weights are indicated. B) Diagram of shell encoding synthetic operon construction. Shell proteins, identified by NCBI Accession numbers, are placed in control of various translation start efficiency within a pET-23b expression vector. B1010 corresponds to ribosome binding site (SEQ ID NO:46).

FIG. 16A-C shows evidence of icosahedral construction. TEM images of unlabeled shells showed thinning in some cases with angles consistent with icosahedral symmetry. In the graph in FIG. 16C, the observed distribution of angles, shown in the histogram bars, suggests a higher representation of 120°-140° and 160°-180° angles, as would be expected for vertices of icosahedral particles projected onto two dimensions. Examples of these angles that are expected to be more highly represented in icosahedral particles are shown in FIG. 16D.

FIG. 17 has two graphs plotting the standard deviation/mean versus shell-protein stoichiometry. The data plotted in the left panel are those calculated with simple ratios of shell units as given Table 2. The right panel graph shows the shell-protein stoichiometry in models 1-5 with BMC-P or with no BMC-P.

FIG. 18 is a graph showing the distribution of shell thickness in nm for 31 different shells. Measurements from 31 different shells were taken from TEM images collected on two different instruments and from two different shell preparations.

FIG. 19 is two images of SDS-PAGE and western blotting of the fractions collected from the purification step for co-purification of GFP-labeled constructs with microcompartment shells. Following ultracentrifugation, the supernatants were separated with sucrose step gradients (20%-70% sucrose in TEMB buffer with 5% step sizes). BMC-containing fractions were pooled and concentrated by ultracentrifugation, then placed over a second sucrose gradient.

### DETAILED DESCRIPTION OF THE PREFERRED EMBODIMENT

### Introduction

Bacterial microcompartments (BMCs) are organelles composed of a protein coat and enzymes that may constitute a (partial) metabolic pathway or cycle, encapsulated by the protein. BMCs separate enzyme-catalyzed reactions from the rest of the cellular environment, allowing the concentration of intermediates in pathways or the sequestering of toxic species from the cellular environment or the enhancement of enzymatic function. Bacterial microcompartments (BMCs) encapsulate functionally related proteins. The bacterial microcompartment shell is composed of multiple paralogs of proteins. BMC shell proteins and the components they encapsulate are typically found in gene clusters (putative operons). The shells of BMCs are composed of multiple paralogs of proteins containing BMC domains pfam00936 and pfam03319. Three types of shell proteins have been identified: single pfam00936 domains (“hexamer”), fusion proteins composed of two pfam00936 domains (“tandem domain”), and single pfam03319 domains (“pentamer”). Hexamer and tandem domain proteins are the major components of known microcompartment shells, while pentamer proteins are presumed to be minor components.

In studying the structure and function of bacterial microcompartments including carboxysomes for CO₂ fixation, we sought a method to rapidly produce engineered microcompartments that could contain desired combinations of enzymes for diverse new synthetic functions. Herein we describe methods, compositions and systems for rapid production and purification of engineered enzyme microcompartments for new synthetic functions and for potential transfer of existing microcompartment functions (including CO₂ fixation machinery) to other organisms.

Many industries including those producing commercial chemicals, pharmaceuticals, and biofuels rely on natural and engineered biosynthetic pathways. Encapsulating and sequestering some of these enzymatic steps in BMCs offers the potential to increase the flux through specific steps in metabolic pathways (as in natural CO₂ fixation pathways) or to prevent cellular toxicity of specific steps in metabolic pathways (as in the processing of aldehyde intermediates in the breakdown of alcohol feedstocks by bacteria). The ability to design and construct new BMCs allows the possibility of achieving these benefits in new natural and engineered biosynthetic pathways. Further, a streamlined synthetic process for construction of BMCs may allow incorporation of, for example, carboxysome-based carbon fixation into new organisms, including plants of interest for biofuels, potentially increasing biomass production in these new host organisms. In addition, engineered microcompartments could be deployed extracellularly, for example to introduce catalytic modules into the environment or for use in precipitation of minerals.

Bacterial microcompartments (BMCs) sequester functionally linked enzymes and metabolic pathways from the cytoplasmic environment by encapsulation inside a selectively permeable protein shell. To develop a system that would allow engineering of new metabolic microcompartments, we screened diverse sets of natural BMC shell proteins, many from organelles of unknown function, for production of BMC shells. From this screen, the seven BMC shell proteins of the halophilic myxobacterium Haliangium ochraceum were found to produce remarkably homogeneous shells in high yield when expressed from a designed synthetic operon in E. coli. These shells were smaller and more uniform than any previously described BMCs, with measured diameters of 39±2 nm. The size and nearly uniform geometry allowed the construction of a precise icosahedral model. This structural model was combined with measurements of stoichiometry, protein structural data, and gold-labeling electron microscopy data to produce a detailed model for shell composition that suggests distinct roles for the seven shell proteins. Further, we found that new proteins could be targeted for encapsulation into the shells by fusion to a predicted targeting peptide sequence. The defined composition and tractability of this system will enable both fundamental studies of the effects of metabolic pathway compartmentalization and the design of new synthetic BMCs. By refactoring shell protein genes from diverse BMC types known only bioinformatically into a synthetic operon, we have introduced a general strategy for the design and construction of novel protein-based synthetic encapsulating structures, carboxysome shells and organelles.

Herein is described a general strategy for production of microcompartment shells in a heterologous host environment. Host species such as E. coli, B. subtilis, or S. cerevisiae are more amenable to laboratory experiments and industrial-scale production than are many prokaryotic organisms that produce BMCs. In particular, many microcompartment containing-containing organisms are difficult to manipulate genetically and pose difficulties in the production and purification of microcompartments. We developed a general strategy for production of microcompartments in heterologous host environments to allow microcompartments including carboxysomes to be produced in bacterial strains that are suitable for genetic manipulation and industrial-scale production, such as E. coli.

### DEFINITIONS

An “expression vector” or “expression cassette” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

By “host cell” is meant a cell that contains an expression vector and supports the replication or expression of the expression vector. Host cells may be prokaryotic cells including but not limited to, E. coli, cyanobacteria including but not limited to, Synechococcus elongatus, or eukaryotic cells including but not limited to, yeast, insect, amphibian, or mammalian cells such as CHO, HeLa and the like, e.g., cultured cells, explants, and cells in vivo.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Amino acid polymers may comprise entirely L-amino acids, entirely D-amino acids, or a mixture of L and D amino acids. The use of the term “peptide or peptidomimetic” in the current application merely emphasizes that peptides comprising naturally occurring amino acids as well as modified amino acids are contemplated.

“Pfam00936 domains” and “Pfam03319 domains” as used herein refer to proteins that are recognized as members of the protein families of those names in the pfam database (Website pfam.sanger.ac.uk). A “hexamer(s)” as used herein is a protein that contains a single pfam00936 domain. A “tandem domains” as used herein is a protein that contains two pfam00936 domains. A “pentamer” as used herein is a protein that contains a pfam03319 domain.

Any “gene” is meant to refer to the polynucleotide sequence that encodes a protein, i.e., after transcription and translation of the gene a protein is expressed. As understood in the art, there are naturally occurring polymorphisms for many gene sequences. Genes that are naturally occurring allelic variations for the purposes of this invention are those genes encoded by the same genetic locus. Thus, any “bacterial microcompartment gene”, “microcompartment gene” as referred to herein is meant to include any polynucleotide that encodes a Pfam00936 domain or Pfam03319 domain protein or variants thereof.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel.

The terms “identical” or percent “identity,” in the context of two or more polypeptide sequences (or two or more nucleic acids), refer to two or more sequences or subsequences that are the same or have a specified percentage of amino acid residues or nucleotides that are the same e.g., 60% identity, preferably 65%, 70%, 75%, 80%, 85%, 90%, 91%, 92%, 93%, 94%, 95%, 96%, 97%, 98%, 99% or 100% identity over a specified region (such as the first 15 amino acids of SEQ ID NOS:20-22), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are typically used.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, polypeptide-nucleic acids (PNAs). Unless otherwise indicated, a particular nucleic acid sequence also encompasses “conservatively modified variants” thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem., 260:2605-2608 (1985); Rossolini et al., Mol. Cell. Probes, 8:91-98 (1994)). The term nucleic acid can be used interchangeably with gene, cDNA, mRNA, oligonucleotide, and polynucleotide.

A “label” or “detectable label” is a composition detectable by spectroscopic, photochemical, biochemical, immunochemical, or chemical means. For example, useful labels include radioisotopes (e.g., ³H, ³⁵S, ³²P, ⁵¹Cr, or ¹²⁵I), fluorescent dyes, electron-dense reagents, enzymes (e.g., alkaline phosphatase, horseradish peroxidase, or others commonly used in an ELISA), biotin, digoxigenin, or haptens and proteins for which antisera or monoclonal antibodies are available (e.g., proteins such as SEQ ID NOS: 23, 25, or 27 can be made detectable, e.g., by incorporating a radiolabel into the protein, and used to detect antibodies specifically reactive with the protein).

### DESCRIPTIONS OF THE EMBODIMENTS

In one embodiment, a bacterial microcompartment (BMC) and metabolic pathway is selected to be synthesized and/or engineered in a host cell. A polynucleotide encoding the bacterial compartment and enzymes in the metabolic pathway can be inserted into a host organism and if needed, expressed using an inducible expression system. When referring to the bacterial compartments or microcompartments, it is meant to include any number of proteins, shell proteins or enzymes (e.g., dehydrogenases, aldolases, lyases, etc.) that comprise or are encapsulated in the compartment.

Naturally existing BMC operons may contain a few or several of these three types of shell proteins. Prior strategies to produce microcompartment shells in heterologus hosts have transformed the host system with the natural operon sequences of the original organism. However, in a natural organism, the required shell proteins may not be placed together on the chromosome, they may be intermixed with enzymes or other proteins, and the ordering and regulatory mechanisms may not be useful in a new host organism.

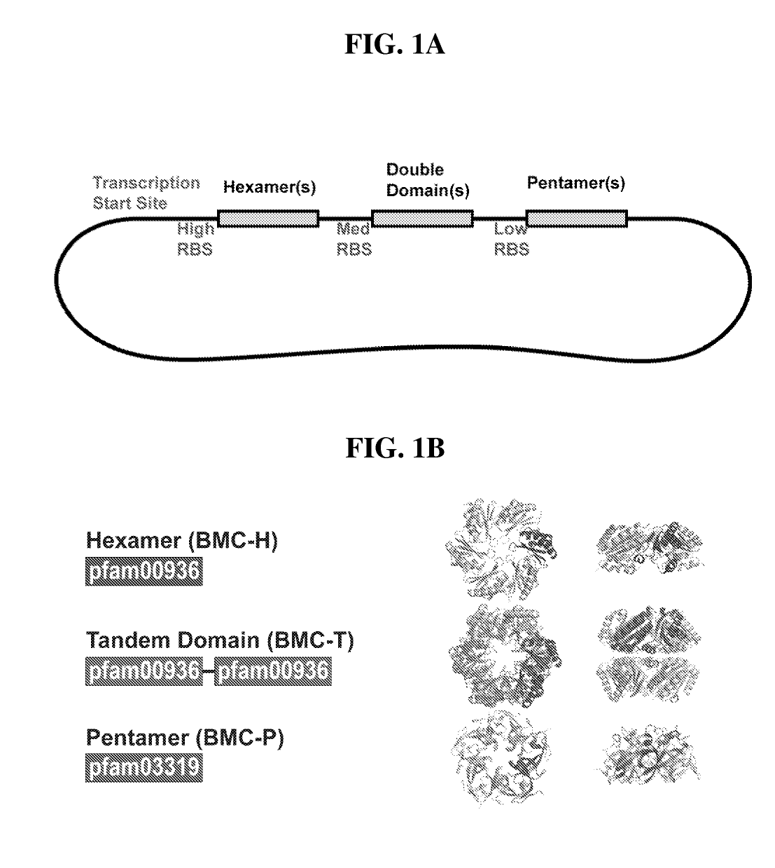

The present approach does not rely on the natural operon sequences. Instead individual BMC components are identified according to their domain types and their roles in the BMC shell construction and their expression levels are adjusted according to these roles by incorporating host-specific ribosomal binding sites in the expression cassette or operon construction (See FIG. 1). Thus, herein is described an experimentally validated strategy and methods to produce a range of naturally occurring microcompartment shells in E. coli and other host organisms.

In one embodiment, polynucleotides encoding bacterial microcompartment shell proteins, are cloned into an appropriate plasmid, inserted into an expression vector, and used to transform cells from any host organism. Suitable host organisms include, but are not limited to, bacteria such as E. coli, B. subtilis, S. cerevisiae, cyanobacteria such as S. elongatus, plants such as Nicotiana tabacum and Camelina sativa, algae, fungi, or other eukaryotic organisms.

In one embodiment, the polynucleotides are in an inducible expression system which maintains the expression of the inserted genes silent unless an inducer molecule (e.g., IPTG) is added to the medium containing the host cell. The expression vector or construct may be a vector for coexpression or in some embodiments, it may be a neutral site vector for insertion into a host genome such as Synechococcous elongatus. The construct may include either inducible transcription elements or may be constitutively expressed in the host organism

Bacterial colonies are allowed to grow after gene expression has begun, or if required, after induction of gene expression. Thus, in some embodiments, expression vectors comprising a promoter operably linked to a heterologous nucleotide sequence or a fragment thereof, that encodes a microcompartment RNA or proteins are further provided. The expression vectors of the invention find use in generating transformed plants, plant cells, microorganisms, algae, fungi, and other eukaryotic organisms as is known in the art and described herein. The expression vector will include 5′ and 3′ regulatory sequences operably linked to a polynucleotide of the invention. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The vector may additionally contain at least one additional gene to be co-transformed into the organism. Alternatively, the additional gene(s) can be provided on multiple expression vectors or cassettes. Such an expression vector is provided with a plurality of restriction sites and/or recombination sites for insertion of the polynucleotide that encodes a microcompartment RNA or polypeptide to be under the transcriptional regulation of the regulatory regions. The expression vector may additionally contain selectable marker genes.

The expression vector will include in the 5′-3′ direction of transcription, a transcriptional initiation region (i.e., a promoter), a cluster of bacterial compartment genes each preceded by a translational initiation site (RBS) specific to the organism and type of shell protein and followed by a translation termination signal (stop codon), and, optionally, a transcriptional termination region functional in the host organism. The regulatory regions (i.e., promoters, transcriptional regulatory regions, ribosomal binding sites and translational termination regions) and/or any targeting sequences may be native or analogous to those found in the host cell or to each other. Alternatively, the regulatory regions and/or the targeting regions may be heterologous to the host cell or to each other. As used herein, “heterologous” in reference to a sequence that originates from a foreign species, or, if from the same species, is modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

In various embodiments, an expression vector further comprising a ribosomal binding site sequence that is specific for the host cell, wherein the ribosomal binding site sequence is placed in the vector adjacent and precedent to a bacterial compartment gene so as to control the translation efficiency of the gene it precedes.

Ribosomal binding sites (RBS) are sequences that precede the coding region of a gene whereby the RBS allows the ribosome to bind the transcript and initiate translation. Ribosomal binding site sequences have been found in various organisms and control and are used herein to vary translation start efficiency in organisms. For example in E. coli, having the sequence of TTTAGAGAAAGAGGAGAAATACTAG (SEQ ID NO:1) is a high ribosomal binding site (RBS) sequence which means that any gene directly following this sequence (i.e., directly 3′- to this sequence) will be translated at a higher rate. This is turn provides for more or greater expression levels of the protein encoded by the gene which follows a high RBS sequence. Likewise other sequences are known to promote a medium or low translation efficiency in E. coli, such as TTTAGAGATTAAAGAGGAGAAATACTAG (SEQ ID NO:2, medium RBS) and TTTAGAGTCACACAGGAAACCTACTAG (SEQ ID NO:3, low RBS).

Therefore, in various embodiments, to produce a BMC shell in a new host organism, a synthetic operon is constructed that contains the desired shell proteins. For each individual protein, an RBS is selected depending on the type. For example, hexamers (BMC-H) are given an RBS with the highest level of translation initiation. Tandem domains are given an RBS at a reduced level of translation intiation (for example, 60% of the predicted value for hexamers). Pentamers are given an RBS with the lowest level of translation initiation (for example, 5% of the predicted value for hexamers. Thus, in some embodiments, the expression vector might further comprise high, medium and/or low ribosomal binding site sequences for a host organism that are inserted in the vector adjacent to and preceding various bacterial compartment genes in the cluster. FIG. 1 shows an example of such a construct where a high RBS sequence precedes the hexamer protein encoding genes, the tandem domain genes are preceded by a medium RBS, and the pentamer genes are preceded by a low RBS sequence. This resulted in high, medium and low expression levels of these proteins after induction.

RBS sequences may be obtained from various sources. They may be designed, for example, by using a calculation to predict translation initiation rates (e.g., Salis, H. M. (2011) The Ribosome Binding Site Calculator, Methods in Enzymology 498: 19-42). RBS sequences may be selected from DNA sequences of natural organisms (e.g. the natural RBS site from the H. neapolitanus shell protein CsoS1C, GATTTTGAATGAGTCTTTATTGAGGAGAGAAGAA (SEQ ID NO:4)). RBS sequences may also be used from databases of biological sequences, including the Registry of Standard Biological Parts (http://partsregistry.org).

The selected microcompartment genes are placed onto the construct using the following general strategy. Single pfam00936 domains and their RBS sequences (“hexamers”) are placed first in the synthetic operon, followed by tandem pfam00936 (“tandem domains”) and their RBS sequences, and finally followed by pfam03319 (“pentamer”) domains and their RBS sequences. Therefore, in various embodiments, an expression vector comprising a transcription start site sequence, one or more nucleic acid sequences for bacterial compartment genes and with ribosomal binding site sequences that are specific for the host cell, wherein the ribosomal binding site sequence is placed in the vector adjacent and directly 5′- to a bacterial compartment gene.

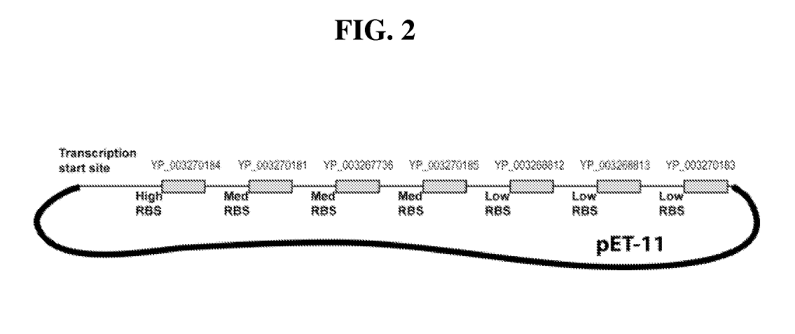

In various embodiments, the synthetic operon contains all the microcompartment genes and their RBS sequences as shown in FIGS. 1 and 2.

In other embodiments, microcompartment genes can be incorporated into multiple expression vectors and/or under multiple promoter control. For example, the specific microcompartment genes encoding H. ochraceum shell proteins could be assembled with one or more expression vectors using one or more different transcription initiation points. The shell proteins need not be placed on a single operon. For example, hexamers could be placed under control of one promoter, tandem domains could be placed under the control of another promoter, and pentamers could be placed under the control of a third promoter. Furthermore, the high RBS sequence and the hexamer gene may be present on one synthetic operon, while the tandem domain genes and its RBS sequence are present on a second synthetic operon and the pentamer gene and its low RBS sequence are presented on a third synthetic operon, or any combination thereof.

Where appropriate, the polynucleotides may be optimized for increased expression in the transformed organism. For example, the polynucleotides can be synthesized using preferred codons for improved expression.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression vector can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). The above list of selectable marker genes is not meant to be limiting. Any selectable marker gene can be used in the present invention.

In another embodiment, it may be beneficial to express the gene from an inducible promoter. The gene product may also be co-expressed with a targeting polypeptide or fragment thereof, such that the polypeptide is in the C-terminal or N-terminal region of any other gene in the construct.

In various embodiments, microcompartment shells may be produced from another organism in a bacterial host cell, such as E. coli, by construction of a synthetic operon as described herein. For example, in one embodiment, the microcompartment shells produced are cyanobacterial carboxysome shell proteins produced in E. coli.

However, in other embodiments, microcompartment shells may be produced in another non-bacterial host organism by construction of a synthetic operon and adjusting the RBS sequences to that host organism. For example, Haliangium ochraceum shells or other types of shells may be produced in cyanobacteria or plants, such as might be done to incorporate Rubsico and carbonic anhydrase to produce a synthetic carboxysome in plants.

In one embodiment, an in-vitro transcription/translation system (e.g., Roche RTS 100 E. coli HY) can be used to produce cell-free microcompartments or expression products.

In some embodiments, it is preferred that the microcompartments expressed in the non-native host organism should provide the host organism enhanced enzymatic activity, enhanced biomass production and CO₂ sequestration abilities, or produce valuable intermediates (Acetyl CoA), or sequester and protect oxygen-sensitive enzymes (engineered or native) or encapsulate reactions that would otherwise be toxic to the cell but however, be non-toxic or have low toxicity levels to humans, animals and plants or other organisms that are not the target.

In some embodiments, the microcompartment proteins are preferably incorporated into the genome of the host microorganism or eukaryote (plant, algae, yeast/fungi) to provide new or enhanced metabolic activity. In some embodiments, the microcompartment proteins are incorporated to provide enhanced carbon fixation and sequestration activity in the plant or organism (i.e., addition of a carboxysome) or produce valuable intermediates (Acetyl CoA), or sequester and protect oxygen-sensitive enzymes (engineered or native) or encapsulate reactions that would otherwise be toxic to the cell.

Genes which encode the enzymes or proteins to carry out these enhanced reactions or activities and which will be encapsulated by the microcompartment may be targeted to the microcompartment by adding encapsulation tags specific for the microcompartment shell. Methods and compositions describing this in greater detail are described previously by some of the inventors in U.S. application Ser. No. 13/367,260 filed on Feb. 6, 2012, published as US-2002/02104590-A1 (“Design and Implementation of Novel and/or Enhanced Bacterial Microcompartments for Customizing Metabolism”), and also described in Lassila, J. K., Bernstein, S. L., Axen S. D., Kinney J. N. and Kerfeld, C. A. Assembly of Robust Bacterial Microcompartment Shells using Building Blocks from an Organelle of Unknown Function. Journal of Molecular Biology in press, both of which are hereby incorporated by reference in their entirety. Such encapsulation tags and the genes encoding the proteins to be encapsulated may be incorporated in the microcompartment expression vector itself or by co-expression of such encapsulation tagged genes which are on a second vector added to the host cell.

In one embodiment, a polynucleotide sequence encoding a targeting peptide or a fragment thereof as described can be inserted into the polynucleotide that encodes a protein of interest in the N-terminus or C-terminus or between functional domains of the proteins, thereby permitting the encapsulation of that protein into the BMC upon expression. Example 3 provides a description of co-expression of encapsulation tagged genes to express proteins which are encapsulated in the microcompartments which are produced in the E. coli host cell. A DNA sequence (SEQ ID NO:24) encoding a polypeptide derived from the N-terminus of aldehyde dehdyrogenase from H. ochraceum was incorporated into an expression construct at the 5′ end of a sequence encoding green fluorescent protein (GFP) for expression in H. ochraceum, which resulted in green fluorescent protein being encapsulated into the shells.

(SEQ ID NO: 24)
ATGGCACTGCGTGAAGATCGTATCGCTGAAATCGTGGAACGTGTCCTGGC
CCGTCTGGATGGCAACTCGGGCTCGTCGGCTGCACCGCATAGCGGCTCTG
GTGCGGGTAGCGGTTCGGGTTCGGGCTCTGGTTCTGGTAGTGGCAGTGGT
AGTGGTTCGGGCTCCGGATCC

In other embodiments, proteins may be incorporated into shells without using encapsulation tags by overexpressing proteins of interest, by electrostatic or hydrophobic or other types of protein-protein interactions that allow association of proteins with microcompartment shell protein, or by fusing proteins to other proteins that associate with shells. For example, an enzyme of interest could be fused to a Rubisco that interacts directly with a shell protein or an enzyme of interest could be directly fused to a shell protein.

In another embodiment, using an encapsulation tag or other approach as listed above, a mechanism is provided for targeting biological molecules that would benefit from being compartmentalized and/or recombining them with other molecules and biological molecules within a bacterial microcompartment shell. This will enable the engineering of new or enhanced bacterial microcompartments. An example strategy is in one embodiment, a carboxysome shell protein is co-expressed with a fluorescent protein-peptide fusion. These protein-peptide fusions can be transferred among organisms (e.g. bacteria, fungi, plants, algae) using basic molecular techniques, followed by directed evolution to optimize phenotype. Alternatively, the modules are stable in solution or can be engineered to be (e.g., via reversible bonds/crosslinks), stable in solution, thus carrying out catalysis in cell free, non-biological systems.

In another embodiment, this allows one to engineer new metabolic modules (essentially organelles of specific function) into a host organism such as bacteria or a plant and provides a new approach to designing and optimizing catalysis in solution. For example, insertion of polynucleotides encoding for the expression of the peptides provided for in SEQ ID NOS: 23 and 27 or for example, at least the localization peptide regions in the polypeptides of SEQ ID NOS: 23 and 27 into the synthetic construct or expression cassette, allows the incorporation or encapsulation of the expression products SEQ ID NOS: 23 and 27 into the microcompartment shell

This strategy allows a fully synthetic and modular approach to design of new microcompartments or for production of existing microcompartments in new host organisms. An additional benefit of this construction method is that it will allow engineering of new pore selectivities by making amino acid substitutions at the perimeter of the pores. The ability to alter the pore selectivities is a crucial step toward being able to develop microcompartments as reaction chambers for any desired new metabolic pathway.

In another embodiment, proteins or other molecules may be introduced into synthetic microcompartments in vitro by dissociating the microcompartment shells in the presence of molecules to be encapsulated and then altering conditions so that the shells re-assemble, thereby trapping the cargo to be encapsulated.

In other embodiments, the constructs for expressing microcompartments described herein may be used for delivery of proteins, biomolecules, drugs or other agents in another organism. In other embodiments, the present constructs and methods may be used to synthetically produce, in bacteria for example, large quantities of the microcompartments encapsulating or incorporating the proteins, biomolecules, drugs or other agents, which after extraction are delivered to another organism needing treatment.

In some embodiments, the synthetic microcompartment described herein comprising shell proteins from H. ochraceum could be used for a broad range of applications in biotechnology in addition to those described above, including as a scaffold for engineered vaccine constructs, as a vehicle for delivery of protein or small molecule drug agents, or as a capsule for stabilizing biocatalyst systems.

In another embodiment, the synthetic microcompartments described herein comprising shell proteins from H. ochraceum or T. elongatus may be used to produce a synthetic carboxysome by incorporation of rubisco and carbonic anhydrase, and by engineering the pores for selective permeability for carbon fixation activity. For example, the expression construct described in Example 5 may be designed to also incorporate the genes for the H. ochraceum enzymes rubisco and carbonic anhydrase, and the genes for selectively permeable pores, with these genes under the correct RBS sequence control. This expression construct could then be inserted into an organism such as bacteria, yeast or a plant such as Tobacco or Camelina.

### Example 1

### Design and Construction of a Synthetic Operon for Expression in E. coli

We have proposed and experimentally validated a strategy to produce a range of naturally occurring microcompartment shells in E. coli and other host organisms. Prior strategies to produce microcompartment shells in heterologus hosts have transformed the host system with the natural operon sequences of the original organism. Our approach does not rely on the natural operon sequences. Individual BMC components are identified according to their domain types and their roles in the BMC shell construction and their expression levels are adjusted according to these roles (FIG. 1).

We surveyed a diverse set of natural BMC gene clusters, many of unknown function, to identify new shell building blocks. The cohort of shell proteins encoded in the genome of the halophilic myxobacterium Haliangium ochraceum³² stood out as an unusual combination of different shell protein types, unlike any other BMC gene cluster. It is atypical in that it is enriched in BMC-T and BMC-P genes relative to BMC-H, which typically occur in multiple copies in BMC gene clusters. Moreover two of the BMC-T genes are part of the small family of BMC-T proteins predicted to form double-stacked dimers of trimers.

Seven BMC gene products were identified from Haliangium ochraceum SMP-2, DSM 14365. DNA sequences were selected to encode the protein sequences while utilizing high-frequency codons from E. coli, the host organism. Thus, the DNA sequences are not those of the native organism, H. ochraceum, even though the protein sequences are the same.

A synthetic construct containing the seven H. ochraceum BMC shell proteins expressed in E. coli resulted in the production of robust shells in high yield. The homogeneity and small size of these shells enabled us to propose a model for shell construction that suggests possible roles for the individual shell proteins. New proteins were targeted to these shells by fusion to peptide encapsulation tags, opening up the possibility of future design of new synthetic microcompartments or three-dimensional scaffolds. The tractability of this system should enable fundamental structural and mechanistic investigations of BMC function.

YP_003270184 Protein
(Hexamer; SEQ ID NO: 6)
MADALGMIEVRGFVGMVEAADAMVKAAKVELIGYEKTGGGYVTAVVRGDVAAVKAATEAGQRAA
ERVGEVVAVHVIPRPHVNVDAALPLGRTPGMDKSA
YP_003270184 Gene
(BMC-H; SEQ ID NO: 7)
ATGGCGGACGCACTGGGTATGATTGAAGTTCGTGGTTTTGTTGGTATGGTGGAAGCGGCGGATG
CTATGGTGAAAGCGGCTAAAGTTGAACTGATTGGTTATGAAAAAACCGGCGGTGGCTACGTGAC
GGCAGTGGTTCGTGGTGATGTCGCAGCAGTTAAGGCAGCTACCGAAGCCGGTCAGCGTGCAGCA
GAACGTGTTGGTGAAGTCGTGGCAGTTCATGTCATCCCGCGTCCGCACGTGAACGTTGATGCAG
CTCTGCCGCTGGGTCGTACGCCGGGTATGGACAAAAGCGCGTAA
YP_003270181 Protein
(BMC-T; SEQ ID NO: 8)
MDHAPERFDATPPAGEPDRPALGVLELTSIARGITVADAALKRAPSLLLMSRPVSSGKHLLMMR
GQVAEVEESMIAAREIAGAGSGALLDELELPYAHEQLWRFLDAPVVADAWEEDTESVIIVETAT
VCAAIDSADAALKTAPVVLRDMRLAIGIAGKAFFTLTGELADVEAAAEVVRERCGARLLELACI
ARPVDELRGRLFF
YP_003270181 Gene
(BMC-T; SEQ ID NO: 9)
ATGGACCACGCTCCGGAACGCTTTGATGCGACCCCGCCGGCAGGTGAACCGGACCGCCCGGCAC
TGGGTGTGCTGGAACTGACCTCAATTGCTCGTGGTATCACCGTTGCGGATGCGGCCCTGAAACG
TGCACCGAGTCTGCTGCTGATGTCCCGCCCGGTCAGCTCTGGCAAGCATCTGCTGATGATGCGT
GGCCAGGTGGCAGAAGTTGAAGAATCAATGATTGCAGCTCGCGAAATCGCTGGTGCAGGTTCGG
GTGCTCTGCTGGATGAACTGGAACTGCCGTATGCGCACGAACAACTGTGGCGCTTTCTGGACGC
ACCGGTGGTTGCAGATGCATGGGAAGAAGACACCGAAAGCGTCATTATCGTGGAAACCGCGACG
GTGTGCGCGGCCATTGATAGTGCCGACGCAGCTCTGAAAACGGCACCGGTCGTGCTGCGTGATA
TGCGCCTGGCCATTGGTATCGCTGGCAAGGCGTTTTTCACCCTGACGGGTGAACTGGCAGACGT
GGAAGCGGCCGCAGAAGTTGTCCGTGAACGTTGCGGTGCACGTCTGCTGGAACTGGCATGTATC
GCACGCCCGGTTGATGAACTGCGTGGCCGCCTGTTTTTCTAA
YP_003267736 Protein
(BMC-T; SEQ ID NO: 10)
MELRAYTVLDALQPQLVAFLQTVSTGFMPMEQQASVLVEIAPGIAVNQLTDAALKATRCQPGLQ
IVERAYGLIEMHDDDQGQVRAAGDAMLAHLGAREADRLAPRVVSSQIITGIDGHQSQLINRMRH
GDMIQAGQTLYILEVHPAGYAALAANEAEKAAPIKLLEVVTFGAFGRLWLGGGEAEIAEAARAA
EGALAGLSGRDNRG
YP_003267736 Gene
(BMC-T; SEQ ID NO: 11)
ATGGAACTGCGTGCTTATACGGTCCTGGATGCCCTGCAGCCGCAACTGGTCGCCTTTCTGCAAA
CGGTGTCAACGGGTTTCATGCCGATGGAACAGCAAGCGAGCGTTCTGGTCGAAATTGCACCGGG
TATCGCTGTCAACCAGCTGACCGACGCAGCACTGAAAGCAACGCGTTGCCAGCCGGGTCTGCAA
ATTGTGGAACGTGCGTATGGCCTGATCGAAATGCATGATGACGATCAGGGTCAAGTTCGTGCAG
CTGGTGACGCAATGCTGGCACACCTGGGTGCACGTGAAGCTGATCGTCTGGCACCGCGTGTGGT
TAGCTCTCAGATTATCACCGGTATTGACGGCCATCAGAGTCAACTGATCAACCGTATGCGCCAC
GGTGATATGATTCAGGCAGGCCAAACGCTGTATATCCTGGAAGTTCATCCGGCAGGTTACGCAG
CACTGGCAGCTAATGAAGCCGAAAAAGCGGCCCCGATTAAGCTGCTGGAAGTCGTGACCTTTGG
TGCATTCGGTCGTCTGTGGCTGGGTGGTGGTGAAGCAGAAATCGCAGAAGCAGCTCGTGCGGCA
GAAGGTGCACTGGCTGGTCTGTCCGGCCGTGATAATCGCGGCTAA
YP_003270185 Protein
(BMC-T; SEQ ID NO: 12)
MSITLRTYIFLDALQPQLATFIGKTARGFLPVPGQASLWVEIAPGIAINRVTDAALKATKVQPA
VQVVERAYGLLEVHHFDQGEVLAAGSTILDKLEVREEGRLKPQVMTHQIIRAVEAYQTQIINRN
SQGMMILPGESLFILETQPAGYAVLAANEAEKAANVHLVNVTPYGAFGRLYLAGSEAEIDAAAE
AAEAAIRSVSGVAQESFRDR
YP_003270185 Gene
(BMC-T; SEQ ID NO: 13)
ATGTCAATCACCCTGCGCACCTATATCTTTCTGGACGCCCTGCAACCGCAACTGGCAACCTTCA
TCGGCAAAACGGCTCGTGGCTTCCTGCCGGTCCCGGGTCAGGCAAGCCTGTGGGTGGAAATTGC
TCCGGGTATTGCGATCAACCGTGTGACCGATGCGGCCCTGAAAGCTACGAAGGTGCAGCCGGCG
GTTCAAGTGGTTGAACGCGCGTATGGCCTGCTGGAAGTTCATCACTTCGATCAGGGCGAAGTCC
TGGCAGCTGGTAGTACCATCCTGGACAAACTGGAAGTTCGTGAAGAAGGTCGCCTGAAGCCGCA
GGTGATGACCCATCAAATTATCCGTGCTGTTGAAGCGTATCAGACGCAAATTATCAACCGCAAT
AGTCAGGGCATGATGATTCTGCCGGGTGAATCCCTGTTTATCCTGGAAACCCAACCGGCAGGTT
ACGCAGTCCTGGCAGCCAATGAAGCCGAAAAAGCAGCTAACGTTCACCTGGTCAATGTGACGCC
GTATGGCGCATTCGGTCGTCTGTACCTGGCCGGCTCAGAAGCAGAAATTGATGCGGCCGCAGAA
GCTGCGGAAGCCGCAATCCGCAGCGTTTCTGGTGTCGCGCAGGAATCGTTTCGTGACCGCTAA
YP_003268812 Protein
(BMC-P; SEQ ID NO: 14)
MYLGRVIGTVVAERKVAGLEGAKLLLVQPLDDALSPVGGVQAAVDTVQAGPDDLVYLVGSREAA
LALTPSFVPVDAAIVGIVDDVHAPERAS
YP_003268812 Gene
(BMC-P; SEQ ID NO: 15)
ATGTATCTGGGTCGTGTGATTGGTACCGTGGTGGCTGAACGCAAAGTGGCGGGTCTGGAAGGCG
CAAAACTGCTGCTGGTGCAACCGCTGGATGACGCACTGAGTCCGGTCGGTGGTGTGCAGGCAGC
AGTTGATACCGTCCAAGCAGGTCCGGATGACCTGGTGTATCTGGTTGGTAGCCGTGAAGCAGCT
CTGGCGCTGACGCCGTCTTTTGTGCCGGTTGATGCGGCCATTGTCGGCATCGTTGATGACGTGC
ATGCACCGGAACGCGCTAGCTAA
YP_003268813 Protein
(BMC-P; SEQ ID NO: 16)
MRLCRVLGSVVATVKHPVYNGLPLMIVQPLDDAGRDAGASFLAVDNVQSGPGDRVLVLTEGGGV
RQILALGDQVPIRSLIVGVVDAVDGVAATGVDDAGGAADSAAAAKSVRADELPADASAAGRGE
YP_003268813 Gene
(BMC-P; SEQ ID NO: 17)
ATGCGTCTGTGTCGTGTTCTGGGCTCCGTCGTCGCCACCGTCAAGCACCCGGTCTACAATGG
TCTGCCGCTGATGATCGTTCAACCGCTGGATGACGCAGGTCGTGATGCAGGCGCTAGTTTTCTG
GCTGTTGATAACGTCCAGTCCGGTCCGGGTGACCGTGTCCTGGTGCTGACCGAAGGTGGTGGTG
TGCGTCAGATTCTGGCACTGGGTGATCAAGTCCCGATTCGCAGCCTGATCGTGGGCGTGGTTGA
TGCAGTGGACGGTGTTGCAGCAACGGGTGTTGATGACGCAGGTGGTGCAGCTGATAGCGCAGCA
GCAGCTAAATCTGTCCGTGCAGATGAACTGCCGGCAGACGCAAGCGCGGCCGGTCGCGGCGAAT
AA
YP_003270183 Protein
(BMC-P; SEQ ID NO: 18)
MVLGKVVGTVVASRKEPRIEGLSLLLVRACDPDGTPTGGAVVCADAVGAGVGEVVLYASGSSAR
QTEVTNNRPVDATIMAIVDLVEMGGDVRFRKD
YP_003270183 Gene
(Pentamer; SEQ ID NO: 19)
ATGGTCCTGGGTAAAGTCGTGGGTACGGTGGTGGCGAGCCGCAAAGAACCGCGCATTGAAGGTC
TGAGCCTGCTGCTGGTCCGTGCCTGCGATCCGGACGGTACCCCGACGGGTGGTGCAGTGGTTTG
TGCAGATGCAGTGGGTGCAGGTGTTGGTGAAGTCGTGCTGTATGCGAGTGGCAGCTCTGCCCGT
CAGACCGAAGTCACGAACAATCGCCCGGTTGATGCAACCATTATGGCTATCGTTGACCTGGTCG
AAATGGGCGGTGATGTGCGTTTTCGCAAAGACTAA

Synthetic operons were produced with these protein sequences. The protein sequences were preceded by ribosomal binding sites with high, medium, and low levels of predicted translation initiation in E. coli. In one example, the RBS sequences immediately 5′- to the gene listed were:

For Hexamer (BMC-H):
(SEQ ID NO: 20)
TCTAGAAATAATTTTGTTTAGAGAAAGAGGAGAAATACTAG
For Tandem domain (BMC-T):
(SEQ ID NO: 21)
TTTAGAGATTAAAGAGGAGAAATACTAG
For Pentamer (BMC-P):
(SEQ ID NO: 22)
TTTAGAGTCACACAGGAAACCTACTAG

These sequences were inserted into the pET-11 expression vector as shown in FIG. 2.

A synthetic operon was constructed as described. Ribosomal binding site sequences from the Community RBS Collection of the Registry of Standard Biological Parts (http://partsregistry.org) were used as follows:

| (SEQ ID NO: 55) | |
|---|---|
| For BMC-H: | TCTAGAGAAAGAGGAGAAATACTAGATG |
| (SEQ ID NO: 56) | |
| For BMC-T: | TCTAGAGATTAAAGAGGAGAAATACTAGATG |
| (SEQ ID NO: 57) | |
| For BMC-P: | TCTAGAGTCACACAGGAAACCTACTAGATG |

The full sequence of the synthetic H. ochraceum operon was as follows and identified as SEQ ID NO:58 with the highlighted regions being the BMC hexamer, tandem domain or pentamer sequences:

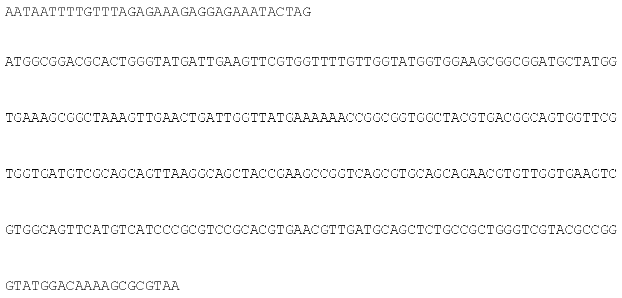

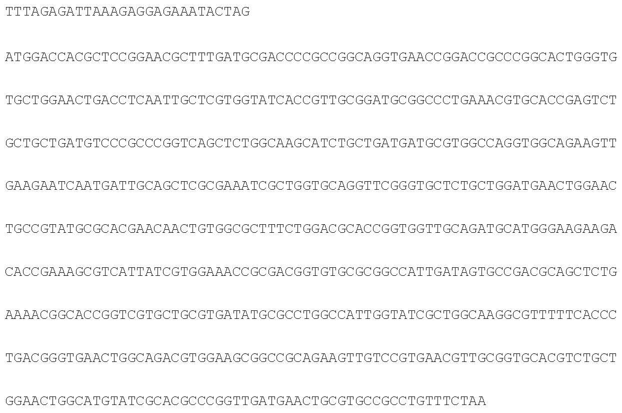

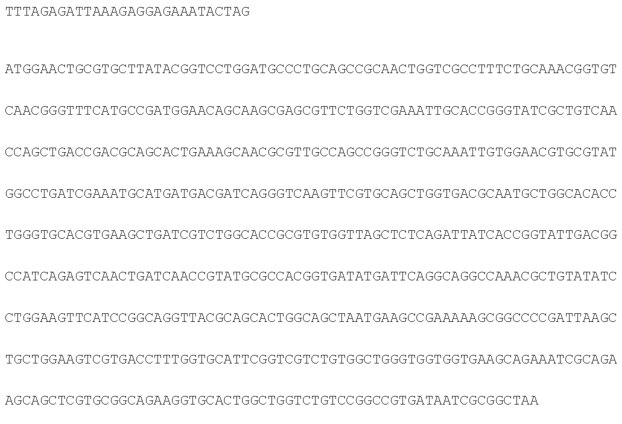

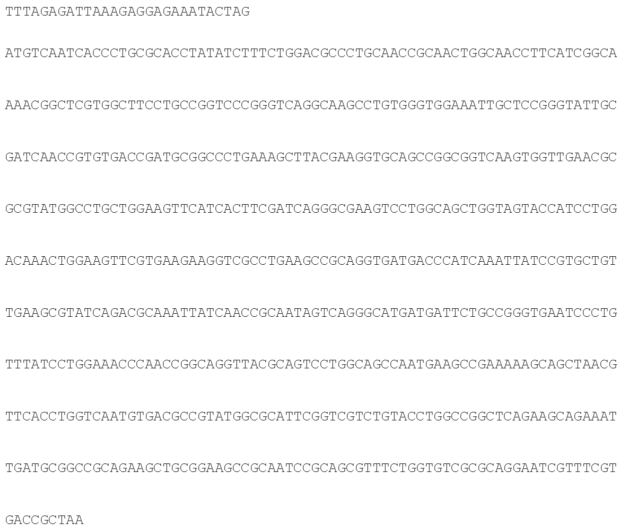

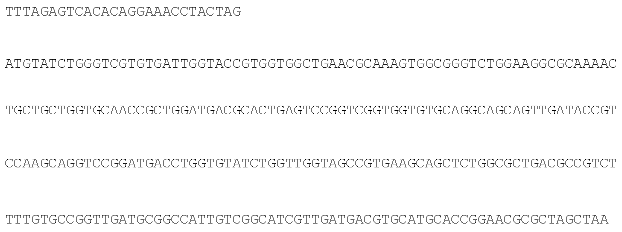

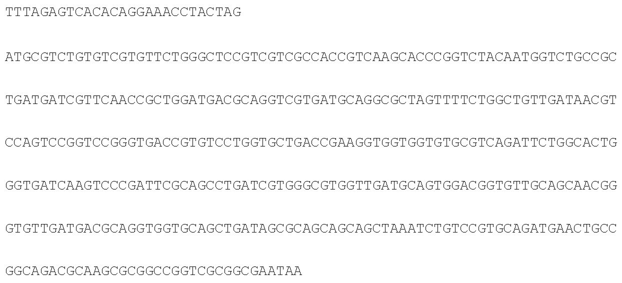

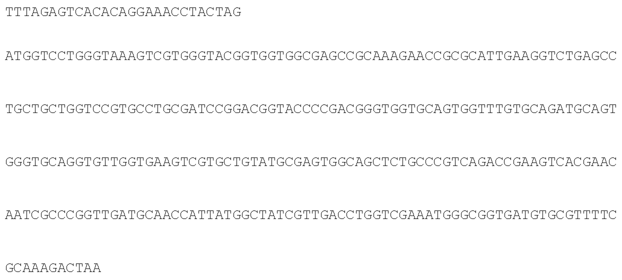

### Example 2

### Expressing and Producing Microcompartment Shells in E. coli

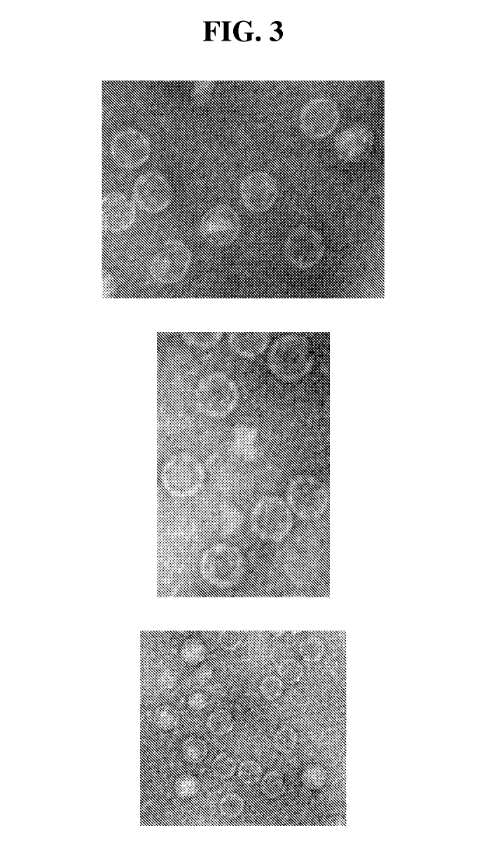

The construct was expressed from an IPTG-inducible pET-11 vector in E. coli BL21(DE3) RIL cells. Expression was induced with 0.4 mM IPTG. The cells were lysed with BPER detergent (Pierce) and centrifuged at low speed (12,000×g). The supernatant was then centrifuged at high speed (60,000×g) to separate assembled microcompartments from soluble proteins. The microcompartments were purified further using density gradient centrifugation. FIG. 3 shows negatively-stained electron microscopy images of purified microcompartments.

The purified shells were analyzed by SDS-PAGE, trypsin digest, and mass spectrometry and found to be composed of the shell proteins from Haliangium ochraceum as designed. Purified shells were incubated on Formvar-coated grids (Electron Microscopy Sciences), negatively stained using ammonium molybdate, and imaged using a Tecnai 12 TEM instrument. FIG. 3. Electron microscopy image of microcompartment shells isolated from E. coli cell extract.

### Example 3

### Using Encapsulation Tags to Incorporate Proteins into Microcompartment Shells

The synthetic operon described above in Example 2 was coexpressed with a second vector, an IPTG-inducible pCOLA-DUET-1 vector, encoding either green fluorescent protein (GFP) with no tag or GFP linked to a tag or full enzyme construct. Microcompartment fractions were isolated as described above.

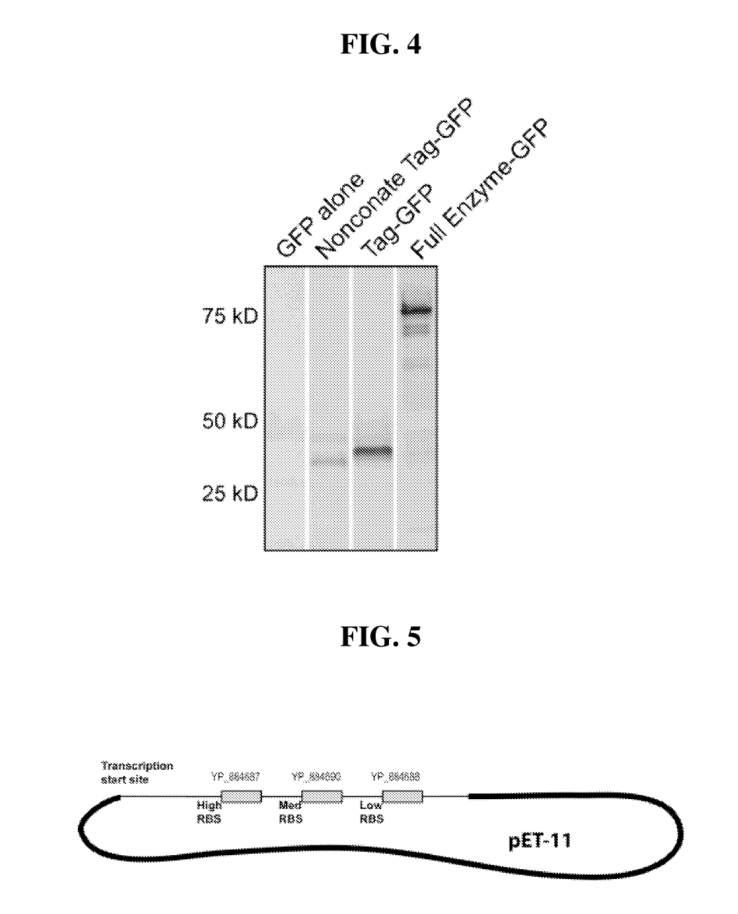

Three types of constructs were tested and were pulled down with the microcompartment shells as shown in FIG. 4.

1. Tag-GFP construct (this uses the Tag sequence that was previously predicted for this organism; the tag is shown in bold). The following protein sequence was fused to the N-terminus of GFP:

(SEQ ID NO: 23)
MALREDRIAEIVERVLARLDGNSGSSAAPHSGSGAGSGSGSGSGSGSG
SGSGSGS.

The DNA sequence used was:

(SEQ ID NO: 24)
ATGGCACTGCGTGAAGATCGTATCGCTGAAATCGTGGAACGTGTCCTGG
CCCGTCTGGATGGCAACTCGGGCTCGTCGGCTGCACCGCATAGCGGCTC
TGGTGCGGGTAGCGGTTCGGGTTCGGGCTCTGGTTCTGGTAGTGGCAGT
GGTAGTGGTTCGGGCTCC.

The sequence of the previously predicted tag is underlined. Additional amino acids were used to extend the linker between the peptide and the GFP because reduced GFP fluorescence was observed without an extended linker. The previously predicted tag was identified as Aldehyde dehydrogenase Nterm (HochDRAFT_{—}00990) with the Accession Number ZP_{—}03875711. The updated Protein Accession Number is YP_{—}003270182 and Locus tag: Hoch_{—}5813.

2. Full Enzyme-GFP construct (the whole enzyme from which the tag was predicted was fused to GFP): The protein sequence is from an aldehyde dehyrdogenase—the same as listed above in #1: Protein accession number YP_{—}003270182; Locus tag: Hoch_{—}5813. The following protein sequence was fused to the N-terminus of GFP with the sequence of the previously predicted tag shown in bold:

(SEQ ID NO: 25)
MALREDRIAEIVERVLARLDGNSGSSAAPHSGSGAPAATAGGASLDIPR
GTLGVYADADAAVNAARRGFAANEALPLRTRQAMIDAMRKVARAHIPEL
ARYAVAETGLGRYEDKLAKNELVIAKTPGPEILAPVAYTGDDGLTLTER
APYGVIGAITPCTNPTETVICNAIGMLSGGNAVVFNVHPSAARVCNWLV
HLLNEAIMSVGGPRDAITSVESPTIDSAQTLMTHAGVRLVVVTGGPGVV
RAAMKSGKKVIAAGPGNPPAVVDETANLAKAAAAIIKGASIDNNIICTA
EKEIVAVASIADELSRLLGQRGALVLGDAQVRALERVVLDGEHVNKEWV
GKDASRIAEQIGLRGHGSDLRLLVCPVDEGHPFVQHELLMPVIGLVRVS
DATEAMATAVRVEHGFCHTAVMHSTHIDRLSAMARVCNASIFVKNDCNL
AGLGLGGEGFTSFTIASPTGEGLTTARDFTRVRRCTLKESFRFVGSGSG
SGSGSGSGSGSGSGS

The DNA sequence used to encode the above fused protein was the following sequence and identified as SEQ ID NO: 26:

ATGGCTCTGCGTGAAGATCGTATCGCTGAAATCGTGGAACGTGTCCTGGC
CCGTCTGGATGGTAACTCCGGCTCGTCGGCTGCCCCGCACAGCGGCTCTG
GTGCGCCGGCGGCCACCGCTGGCGGTGCGAGCCTGGATATTCCGCGTGGC
ACGCTGGGTGTGTATGCAGATGCTGACGCAGCTGTTAACGCAGCACGTCG
CGGTTTTGCAGCTAATGAAGCCCTGCCGCTGCGTACCCGTCAGGCAATGA
TTGATGCAATGCGTAAAGTTGCGCGCGCCCATATCCCGGAACTGGCACGT
TATGCAGTGGCTGAAACCGGCCTGGGTCGCTACGAAGATAAACTGGCTAA
GAACGAACTGGTTATTGCGAAGACGCCGGGCCCGGAAATCCTGGCACCGG
TGGCATATACGGGCGATGACGGTCTGACCCTGACGGAACGCGCCCCGTAC
GGCGTTATTGGTGCAATCACCCCGTGCACGAACCCGACCGAAACGGTGAT
TTGTAATGCCATCGGTATGCTGTCAGGCGGTAACGCAGTGGTTTTCAATG
TGCATCCGTCGGCGGCCCGTGTTTGCAACTGGCTGGTCCACCTGCTGAAT
GAAGCTATTATGAGTGTTGGCGGTCCGCGCGATGCCATTACCAGTGTCGA
ATCCCCGACGATCGACTCCGCTCAAACCCTGATGACGCACGCGGGCGTTC
GTCTGGTCGTGGTTACCGGCGGTCCGGGCGTCGTGCGTGCAGCTATGAAA
TCAGGTAAAAAGGTTATCGCGGCGGGTCCGGGCAACCCGCCGGCGGTTGT
GGATGAAACCGCTAATCTGGCGAAAGCAGCTGCGGCCATTATCAAGGGTG
CATCGATCGATAACAATATTATCTGTACCGCGGAAAAAGAAATTGTCGCG
GTGGCCAGCATCGCAGACGAACTGTCTCGTCTGCTGGGTCAGCGTGGTGC
GCTGGTCCTGGGCGATGCTCAAGTGCGTGCGCTGGAACGCGTGGTTCTGG
ACGGCGAACATGTGAACAAAGAATGGGTTGGCAAGGATGCCAGCCGTATT
GCAGAACAGATCGGTCTGCGTGGCCACGGTTCTGATCTGCGTCTGCTGGT
CTGCCCGGTGGACGAAGGCCATCCGTTTGTCCAACACGAACTGCTGATGC
CGGTGATTGGTCTGGTTCGTGTCAGCGATGCCACCGAAGCAATGGCTACG
GCGGTGCGCGTTGAACATGGCTTTTGTCACACCGCGGTGATGCATAGTAC
GCACATTGACCGTCTGTCCGCGATGGCGCGTGTGTGCAATGCGTCTATCT
TCGTCAAAAACGATTGTAATCTGGCAGGTCTGGGTCTGGGCGGTGAAGGT
TTTACCTCATTCACGATCGCATCGCCGACCGGTGAAGGTCTGACCACGGC
ACGTGATTTCACCCGCGTTCGTCGCTGCACGCTGAAAGAATCTTTTCGCT
TCGTGGGTTCTGGTAGTGGCTCTGGTTCAGGCAGTGGCTCAGGCTCAGGC
TCGGGCAGTGGTTCT

3. Noncognate Tag-GFP construct (the whole enzyme from which the tag was predicted was fused to GFP): In this case, a different tag formerly identified as Aldehyde dehydrogenases Cterm (Ckl_{—}1074; YP_{—}001394464) and (Ckl_{—}1076, YP_{—}001394466). This tag is a non-cognate tag, in other words it was predicted to associate with shells from a different organism. There appears to still be some association with these shells. In this case, the protein sequence was fused to the C-terminus of GFP.

Protein sequence fused to C-terminus of GFP with the tag in bold:

(SEQ ID NO: 27)
GSGSGSGSGSEPEDNEDVQAIVKAIMAKLNL

DNA sequence fused to the 3′ end of the DNA encoding GFP:

(SEQ ID NO: 28)
CTCGAGGGCAGCGGCAGCGGCAGCGGCAGCGGCTCTGAACCGGAAGACAA
TGAAGATGTGCAGGCAATCGTGAAAGCAATTATGGCTAAACTGAACCTG

FIG. 4 shows western blots of these microcompartment fractions with anti-GFP antibody, indicating that GFP is associated with the microcompartment shells when encapsulation tags are present.

### Example 4

### Microcompartment Shells from Mycobacterium Smegmatis Produced in E. coli

Shell proteins from Mycobacterium smegmatis were used to construct a new synthetic operon as shown in FIG. 5.

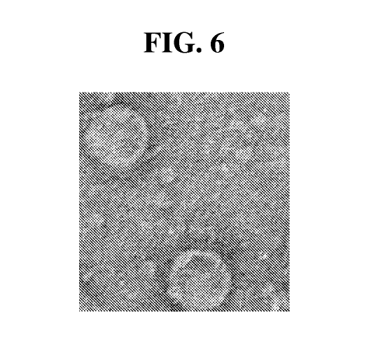

The construct was placed under the control of the IPTG-inducible pET-11 vector and expressed in BL21(DE3)RIL cells. Cells were induced with 0.4 mM IPTG and harvested by centrifugation. Cells were lysed using BPER-2 detergent with sonication. Cell suspensions were clarified using a low speed centrifugation step (10,000×g). The supernatant was centrifuged at high speed (60,000×g) to pellet shell assemblies. Further purification with sucrose density gradient centrifugation and agarose gel electrophoresis (0.2%) was performed. Shells were visualized using negatively-stained electron microscopy with a JEOL 1200EX TEM instrument (FIG. 6).

The operon was designed using the sequences as follows:

RBS DNA sequence:
(SEQ ID NO: 29)
TTTTGTTTAGAGAAAGAGGAGAAATACTAG
YP_884687
Protein sequence:
(SEQ ID NO: 30)
MSSNAIGLIETKGYVAALAAADAMVKAANVTITDRQQVGDGLVAVIVTG
EVGAVKAATEAGAETASQVGELVSVHVIPRPHSELGAHFSVSSK
DNA sequence:
(SEQ ID NO: 31)
ATGAGCAGCAATGCAATCGGTCTGATCGAAACGAAAGGCTATGTGGCGG
CACTGGCAGCGGCGGATGCAATGGTGAAGGCAGCAAATGTCACCATTAC
GGATCGTCAGCAAGTTGGCGACGGTCTGGTGGCGGTTATCGTCACCGGC
GAAGTGGGTGCCGTTAAAGCGGCCACCGAAGCAGGCGCTGAAACGGCAA
GTCAAGTGGGTGAACTGGTGTCCGTTCATGTCATTCCGCGTCCGCACAG
CGAACTGGGTGCACATTTTAGCGTTAGCTCTAAGTAA
RBS DNA Sequence:
(SEQ ID NO: 32)
TTTAGAGATTAAAGAGGAGAAATACTAG
YP_884690
Protein sequence:
(SEQ ID NO: 33)
MAELRSFIFIDRLQPQTMSYLGTWIKGALPRANMAAQIIEVAPGLDIEG
VTDVALKHAEVKAGILVVERQFGYLEFHGETGAVKAAADAALDYLGGDP
DAAVRPEILASRIISSIDHQHAFLINRNKIGSMVLPGESLFVLEVAPAS
YAILATNEAEKAADVKVVDFRMIGATGRVYLSGTEADVRQAADAARDAL
AVLQGA
DNA sequence:
(SEQ ID NO: 34)
ATGGCCGAACTGCGTAGCTTCATTTTCATTGACCGCCTGCAACCGCAAA
CGATGTCCTATCTGGGCACCTGGATTAAGGGTGCTCTGCCGCGTGCGAA
CATGGCGGCCCAGATTATCGAAGTTGCCCCGGGCCTGGATATTGAAGGT
GTTACCGACGTCGCCCTGAAACATGCAGAAGTCAAGGCTGGCATCCTGG
TGGTTGAACGCCAATTTGGTTATCTGGAATTTCATGGCGAAACGGGTGC
GGTGAAAGCAGCTGCGGATGCCGCACTGGACTACCTGGGTGGTGATCCG
GACGCTGCAGTTCGTCCGGAAATTCTGGCCTCTCGCATTATCAGCTCTA
TCGATCATCAGCACGCATTTCTGATTAACCGTAATAAGATCGGCAGTAT
GGTCCTGCCGGGTGAATCCCTGTTCGTGCTGGAAGTTGCTCCGGCGAGC
TATGCGATTCTGGCGACCAATGAAGCGGAAAAAGCCGCAGATGTTAAGG
TCGTGGACTTTCGTATGATCGGTGCAACCGGTCGTGTCTACCTGTCGGG
CACGGAAGCTGATGTGCGTCAGGCTGCAGATGCAGCACGCGACGCACTG
GCAGTGCTGCAAGGTGCCTAA
RBS:
(SEQ ID NO: 35)
TTTAGAGTCACACAGGAAACCTACTAG
YP_884688
Protein sequence:
(SEQ ID NO: 36)
MLRATVTGNVWSTRRIEGIPAGAFLEVEVEGTGSRMIAFDVLGSGVGEH
VLIAQGSVASSWFTGTPPPIDALIIGSIDTRSDSNPAE
DNA Sequence:
(SEQ ID NO: 37)
ATGCTGCGTGCTACCGTTACCGGCAATGTCTGGTCTACCCGTCGTATCG
AAGGCATCCCGGCTGGTGCTTTTCTGGAAGTGGAAGTCGAAGGCACCGG
TTCACGTATGATTGCCTTTGATGTCCTGGGCTCGGGTGTGGGCGAACAT
GTTCTGATCGCGCAGGGTAGCGTTGCCAGCTCTTGGTTCACCGGTACGC
CGCCGCCGATTGACGCACTGATTATCGGTAGTATCGATACGCGCAGTGA
CTCCAACCCGGCTGAATAA

### Example 5

### Production of a Synthetic Metabolic Microcompartment

To produce a synthetic microcompartment to carry out a new metabolic pathway, we designed a strategy to coexpress the H. ochraceum shell proteins with Rubisco and carbonic anhydrase for use in cyanobacteria and ultimately for use in transforming plants for increased carbon fixation efficiency. The H. ochraceum shell proteins are placed in a neutral site vector for insertion into the genome of Synechococcus elongatus PCC 7942 and used to transform S. elongatus. Rubisco and carbonic anhydrase sequences are fused to encapsulation tags and coexpressed with the shell proteins. The construct, and variants thereof, particularly of amino acids at pore sites implicated in determining selectivity of pore permeability, are tested for their ability to increase growth of cyanobacterial strains that are dependent on high CO₂ concentrations.

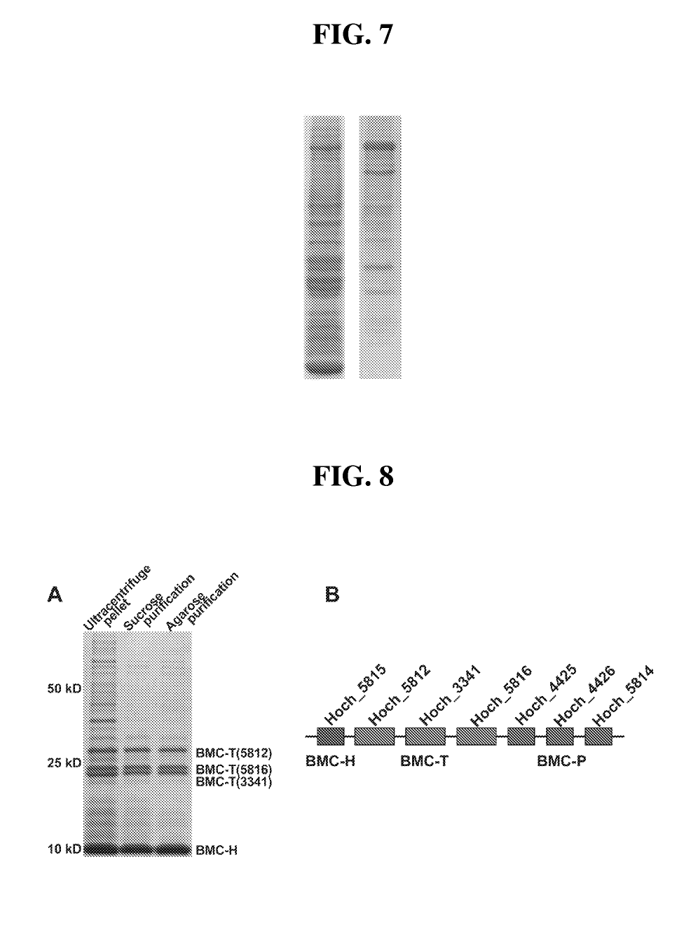

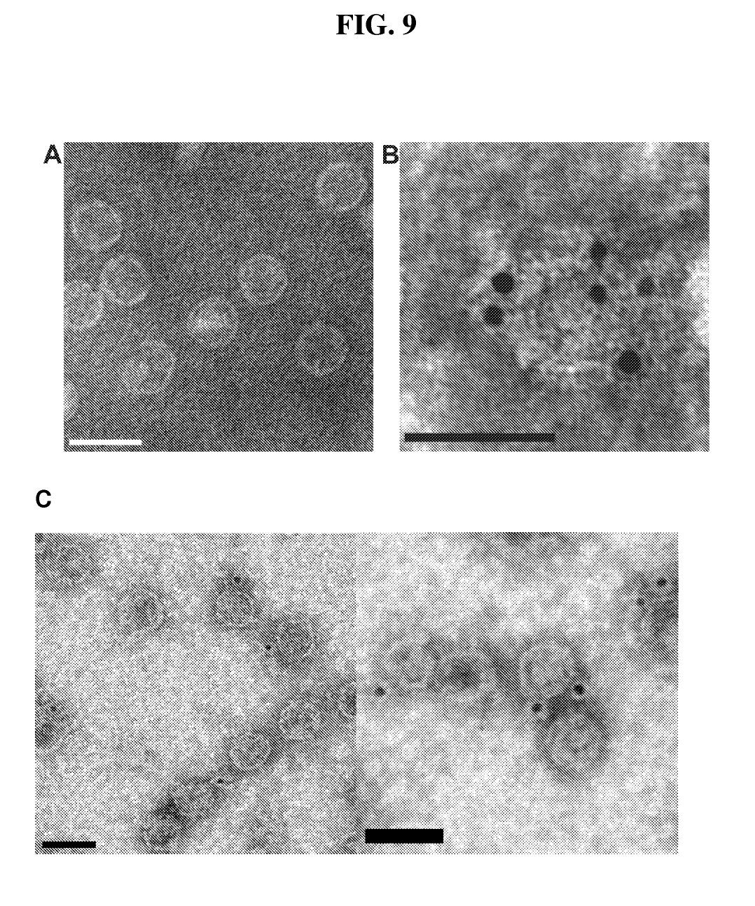

In an initial test, the shell proteins from Haliangium ochraceum (as described in prior example) were co-expressed with a Rubisco variant in E. coli and Rubisco was found to associate with the shells (FIG. 7).

FIG. 7 shows the capture of the non-native enzyme Rubisco by the microcompartment shells produced by H. ochraceum. (Left) SDS-PAGE gel showing microcompartment shell proteins pulled down by ultracentrifugation step. (Right) Western blot with anti-Rubisco indicating that Rubisco was pulled down with the shells. Further experiments (not shown) indicate that Rubisco co-purifies with the shells in sucrose density gradient ultracentrifugation steps.

Rubisco Protein sequence:
(SEQ ID NO: 38)
MAAKKYSAGVKEYRQTYWTPDYVPLDTDLLACFKVTPQPGVPREEAAAA
VAAESSTGTWTTVWTDLLTDMDYYKGRCYRIEDVPGDDESFYAFIAYPL
DLFEEGSVTNVLTSLVGNVFGFKALRALRLEDIRFPMAYVKTCAGPPHG
IQVERDKMNKYGRPLLGCTIKPKLGLSAKNYGRAVYECLRGGLDFTKDD
ENINSQPFQRWRDRFEFVAEAVEKAEAETGERKGHYLNVTAPTPEEMYK
RAEFAKELGAPIIMHDYITGGFTANTGLAKWCRDNGVLLHIHRAMHAVI
DRHPNHGIHFRVLAKCLRLSGGDHLHTGTVVGKLEGDRASTLGYIDLLR
ESFIPEDRSRGIFFDQDWGSMPGVFAVASGGIHVWHMPALVSIFGDDSV
LQFGGGTLGHPWGNAAGAAANRVALEACVQARNEGRDIEKEGKDILTEA
AKHSPELAIALETWKEIKFEFDTVDKLDTQ
Rubisco DNA sequence:
(SEQ ID NO: 39)
CATATGGCAGCGAAAAAATACAGCGCAGGCGTGAAAGAATACCGCCAAA
CCTACTGGACTCCCGATTATGTTCCCCTCGATACGGACCTCCTGGCCTG
CTTTAAAGTTACCCCCCAGCCAGGTGTGCCCCGCGAAGAGGCAGCAGCT
GCAGTCGCAGCAGAAAGCTCGACTGGCACCTGGACCACGGTTTGGACCG
ACCTGCTCACGGATATGGACTACTATAAGGGTCGCTGTTACCGCATCGA
GGATGTGCCTGGCGATGACGAAAGCTTTTACGCTTTCATTGCATATCCA
TTGGATCTGTTTGAAGAGGGCTCGGTTACTAACGTGCTGACCAGTCTCG
TCGGTAATGTTTTTGGCTTCAAAGCCCTGCGCGCGCTCCGCTTGGAAGA
TATCCGCTTCCCGATGGCCTACGTGAAGACCTGCGCAGGTCCCCCGCAT
GGCATTCAAGTCGAACGCGATAAAATGAACAAGTATGGTCGCCCCTTGC
TGGGCTGCACGATCAAACCGAAGCTGGGTCTCTCGGCTAAAAATTACGG
CCGCGCCGTGTATGAATGTTTGCGCGGCGGTCTGGATTTTACCAAGGAT
GACGAGAACATTAATAGCCAGCCCTTCCAACGCTGGCGCGATCGCTTTG
AATTTGTGGCGGAAGCTGTCGAGAAAGCAGAAGCCGAGACGGGCGAGCG
CAAGGGCCATTACCTGAACGTCACCGCGCCTACGCCAGAAGAGATGTAT
AAACGCGCTGAATTTGCAAAGGAGCTCGGCGCTCCCATCATTATGCACG
ATTACATCACCGGCGGTTTCACTGCCAACACCGGTTTGGCGAAATGGTG
CCGCGACAATGGCGTTCTCTTGCACATCCATCGCGCCATGCACGCGGTG
ATTGATCGCCACCCGAATCATGGCATCCACTTTCGCGTCCTCGCGAAAT
GTTTGCGCCTGAGTGGCGGTGATCACTTGCATACGGGTACTGTGGTCGG
CAAGTTGGAAGGTGACCGCGCCAGCACCCTGGGCTATATTGATCTGCTC
CGCGAGAGCTTTATCCCGGAAGATCGCTCGCGCGGCATCTTTTTCGATC
AGGACTGGGGCTCGATGCCCGGTGTGTTCGCAGTCGCTAGTGGTGGTAT
CCATGTGTGGCACATGCCGGCGCTCGTCAGTATTTTTGGCGATGACAGC
GTGCTGCAGTTCGGTGGTGGTACCCTCGGTCATCCTTGGGGTAACGCTG
CAGGTGCAGCAGCTAATCGCGTCGCTCTGGAGGCATGCGTTCAAGCCCG
CAACGAAGGTCGCGACATCGAAAAAGAGGGCAAGGATATTCTCACTGAG
GCAGCCAAGCACAGCCCGGAACTCGCAATCGCCTTGGAAACGTGGAAAG
AGATTAAGTTTGAATTTGATACGGTCGACAAACTGGATACTCAATAG

### Example 6

### Microcompartment Shells from T. elongatus Shell Proteins Produced in E. coli

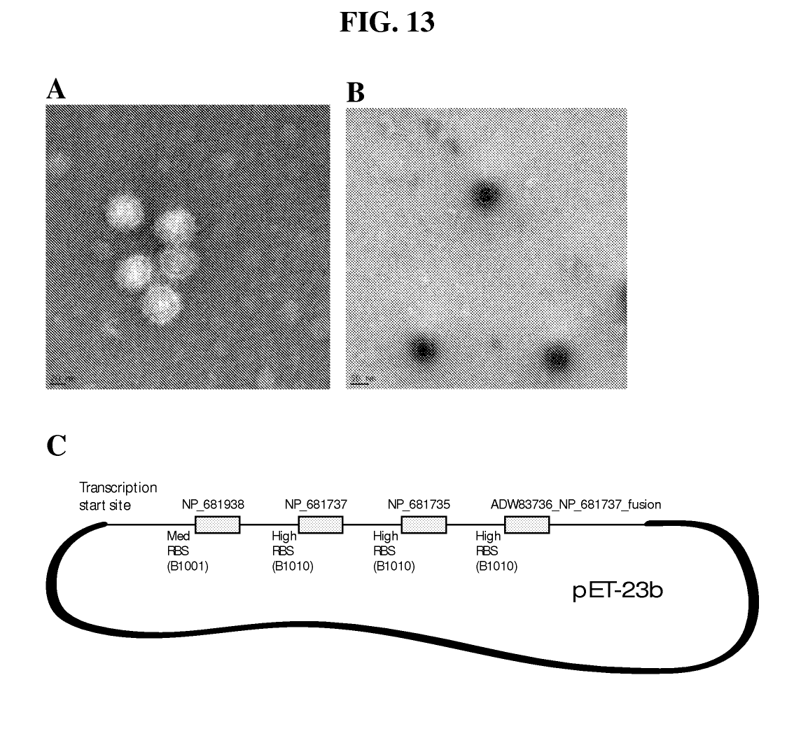

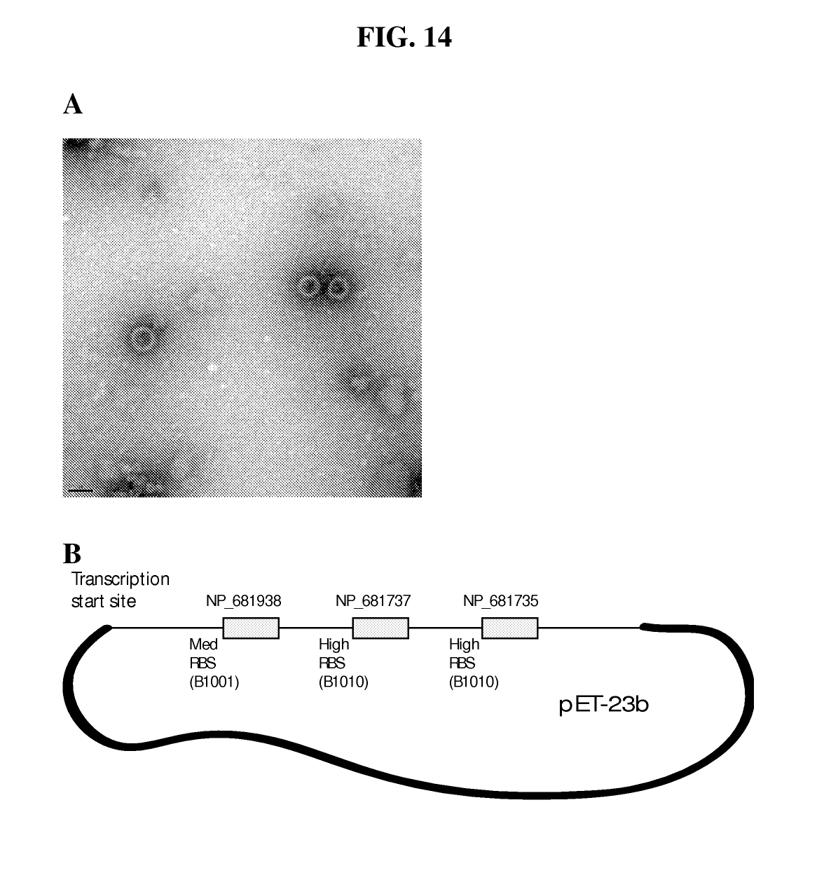

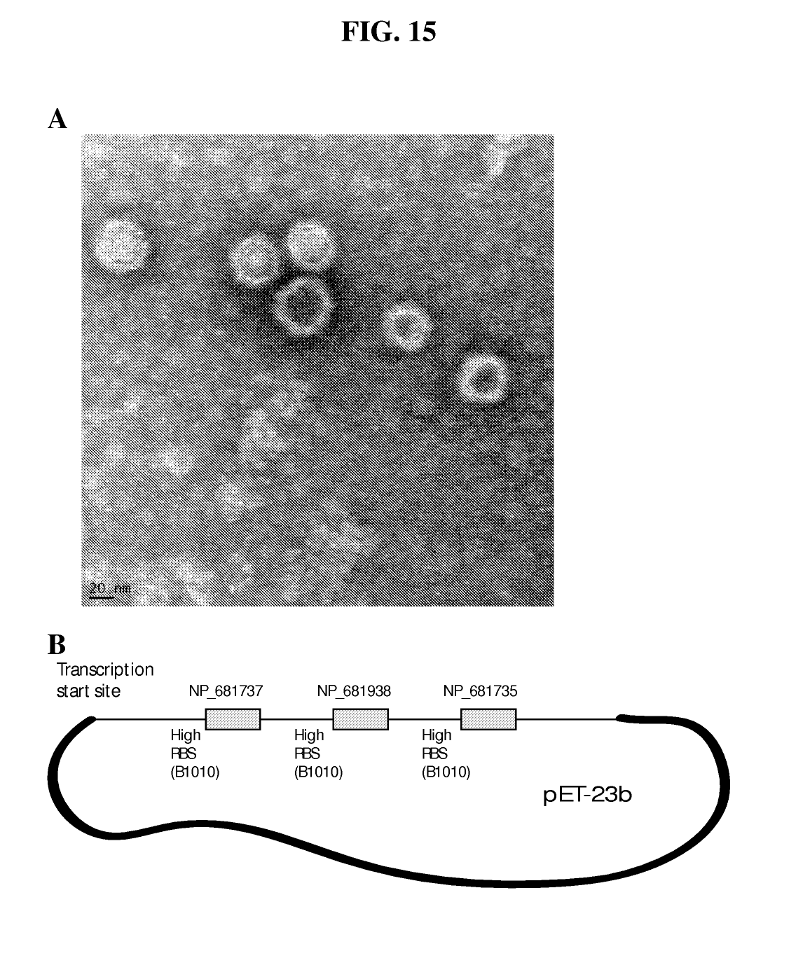

FIGS. 13-15 show synthetic micrcompartments (shells) produced by the expression of shell proteins (CcmK(BMC-H), CcmO(BMC-O), and CcmL(BMC-P)) from T. elongatus along with diagrams of the various synthetic operons used to express the proteins contained in the shells. All constructs were placed under the control of a pET23b IPTG-inducible vector and expressed in BL21(DE3) cells. Cells were induced with IPTG and harvested by centrifugation. Cells were lysed and cell suspensions were clarified using a low speed centrifugation step. The supernatant was centrifuged at high speed to pellet shell assemblies. Further purification with sucrose density gradient centrifugation was performed. Shells were visualized using negatively-stained electron microscopy with a JEOL 1200EX TEM instrument. FIG. 13 shows GFP labeled shells produced by fusing GFP to the hexamer, CcmK2(BMC-H), and expressing this fusion protein on the same vector as the hexamer, pentamer, and tandem domain proteins; a diagram of this expression construct is shown in FIG. 13C. The construct was expressed as above and shells were purified as above. The electron microscopy image shown in FIG. 13A shows projections consistent with GFP attached to the surface of microcompartment shells. FIG. 14A depicts TEM of unlabeled shells composed of only the shell proteins CcmO(BMC-T), CcmK2(BMC-H), and CcmL(BMC-P) from T. elongatus. These represent carboxysome shells containing the minimum complement of BMC domain proteins required to form a shell: one hexameric BMC domain protein (pfam00936), one tandem BMC domain (pfam00936), and one pentameric BMC domain protein (pfam03319), CcmK2(BMC-H), CcmO(BMC-T), and CcmL(BMC-P), respectively. FIG. 15 shows shells purified using ammonium sulfate precipitation purification and an SDS-PAGE gel showing different shell containing fractions of the ammonium sulfate precipitation purification. The samples that contained the shells shown in FIG. 15 were of greater purity than the samples that contained the shells shown in FIGS. 13 and 14, which had a moderate amount of impurities. In the synthetic operon shown in FIG. 15C the shell protein encoding genes are arranged in the following order: ccmK2(BMC-H), ccmO(BMC-T), and ccmL(BMC-P). This arrangement differs from the arrangements shown in FIGS. 13C and 14B; on those synthetic operons the shell protein encoding genes are arranged as follows: ccmO(BMC-T), ccmK2(BMC-H), and ccmL(BMC-P). All of the shells depicted in FIGS. 13-15 were found to have diameters of approximately 50 nm.

CcmK2 Hexamer (BMC-H) carbon dioxide concentrating mechanism protein [Thermosynechococcus elongatus BP-1:NP_{—}681737]

(SEQ ID NO: 40)
MPIAVGMIETRGFPAVVEAADAMVKAARVTLVGYEKIGSGRVTVIVRGDV
SEVQASVAAGVDSAKRVNGGEVLSTHIIARPHENLEYVLPIRYTEAVEQF
RN

carbon dioxide concentrating mechanism protein [Thermosynechococcus elongatus BP-1: GI:22298490(codon optimized)]

(SEQ ID NO: 41)
ATGCCAATTGCCGTGGGTATGATTGAAACCCGTGGTTTTCCAGCCGTGGT
GGAAGCGGCCGATGCCATGGTGAAAGCCGCGCGTGTTACCCTGGTGGGTT
ACGAGAAAATCGGTAGTGGTCGTGTGACCGTGATTGTGCGTGGTGATGTG
AGTGAAGTGCAAGCCAGTGTTGCGGCCGGTGTGGATAGTGCCAAACGTGT
GAATGGTGGCGAAGTGCTGAGTACCCATATCATTGCCCGTCCACATGAAA
ATCTGGAATACGTGCTGCCAATCCGTTACACCGAAGCCGTTGAACAATTT
CGTAAT

CcmO Tandem Domain (BMC-T)

hypothetical protein tll1148 [Thermosynechococcus elongatus BP-1:NP_{—}681938]

(SEQ ID NO: 42)
MERRDDFTDLALGLVSVQSFPAIVGIADHMLKSSDVLLVGYEKIGGGHCT
AIVRGRIADVRLAVEEGAERAQQFGQELSTLVIPRPDPNLEKILPIGSLL
AQIASKSRGHRLSSHAVGLLETRGFPAMVGAADAMLKAADVMLTAYETIG
AGLCTAIIRGTASNTAIALEAGMAEADRIGELHAVMLVPRPLEDLDQSLP
LAPALQRELQPLRLPLTLKQKETEPLALQGAAQASVAVEAAAERVPVDPP
ANP

hypothetical protein tll1148 [Thermosynechococcus elongatus BP-1: GI:22298691 (codon optimized)]

(SEQ ID NO: 43)
ATGGAACGTCGTGATGATTTTACCGATCTGGCCCTGGGTCTGGTGAGTGT
GCAAAGTTTTCCGGCCATCGTGGGTATCGCCGATCATATGCTGAAGAGTA
GTGATGTGCTGCTGGTTGGTTACGAAAAAATCGGTGGTGGCCATTGCACG
GCGATCGTGCGTGGTCGCATTGCGGACGTGCGCCTGGCGGTGGAAGAGGG
TGCCGAACGTGCCCAACAATTTGGTCAAGAACTGAGTACCCTGGTGATTC
CACGTCCAGATCCAAATCTGGAAAAGATTCTGCCGATTGGTAGTCTGCTG
GCGCAAATCGCGAGTAAAAGTCGTGGTCATCGTCTGAGCAGTCATGCCGT
TGGCCTGCTGGAGACCCGTGGTTTCCCAGCCATGGTGGGTGCGGCGGATG
CCATGCTGAAAGCGGCCGATGTGATGCTGACGGCCTACGAGACCATTGGT
GCCGGTCTGTGTACCGCCATCATTCGCGGCACGGCCAGTAATACCGCGAT
TGCCCTGGAAGCCGGTATGGCCGAAGCCGATCGTATTGGTGAACTGCATG
CGGTTATGCTGGTGCCACGCCCGCTGGAAGACCTGGATCAAAGTCTGCCG
CTGGCCCCAGCCCTGCAACGCGAGCTGCAACCACTGCGTCTGCCACTGAC
CCTGAAACAAAAAGAAACCGAGCCACTGGCGCTGCAAGGTGCCGCCCAAG
CCAGTGTGGCCGTTGAAGCCGCCGCCGAGCGTGTTCCAGTTGATCCGCCA
GCCAATCCA

CcmL Pentamer (BMC-P)

carbon dioxide concentrating mechanism protein [Thermosynechococcus elongatus BP-1: NP_{—}681735]

(SEQ ID NO: 44)
MKIARVCGTVTSTQKEDTLTGVKFLVLQYLGEDGEFLPDYEVAADTVGAG
QDEWVLVSRGSAARHIINGTDKPIDAAVVAIIDTVSRDNYLLYSKRTQY

carbon dioxide concentrating mechanism protein [Thermosynechococcus elongatus BP-1: GI:22298488(codon optimized)]

(SEQ ID NO: 45)
ATGAAAATTGCCCGTGTGTGTGGTACCGTGACCAGTACCCAAAAAGAAGA
TACCCTGACCGGTGTGAAGTTTCTGGTGCTGCAATACCTGGGTGAAGATG
GTGAATTTCTGCCAGATTACGAAGTTGCGGCGGACACCGTTGGTGCCGGT
CAAGATGAATGGGTGCTGGTGAGTCGCGGTAGTGCCGCCCGTCACATTAT
CAATGGCACCGATAAACCAATTGATGCCGCCGTGGTGGCCATTATTGATA
CCGTTAGTCGTGATAATTACCTGCTGTATAGTAAACGTACCCAGTACTAA

B1010 ribosome binding site used in the expression constructs in example 6.

(SEQ ID NO: 46)
T TTAAGA AGGAGA TATACC

B1001 ribosome binding site used in the expression constructs in example 6.

(SEQ ID NO: 47)
GG CTAACA TAGGGT GGATCT

SFGFP

Expression product of synthetic construct sfGFP_iGEM gene, complete cds [synthetic contstruct, KF410612]

(SEQ ID NO: 48)
MRKGEELFTGVVPILVELDGDVNGHKFSVRGEGEGDATNGKLTLKFICT
TGKLPVPWPTLVTTLTYGVQCFARYPDHMKQHDFFKSAMPEGYVQERTI
SFKDDGTYKTRAEVKFEGDTLVNRIELKGIDFKEDGNILGHKLEYNFNS
HNVYITADKQKNGIKANFKIRHNVEDGSVQLADHYQQNTPIGDGPVLLP
DNHYLSTQSVLSKDPNEKRDHMVLLEFVTAAGITHGMDELYK

Synthetic construct sfGFP_iGEM gene, complete cds [synthetic construct, GI:532528632]

(SEQ ID NO: 49)
ATGCGTAAAGGCGAAGAGCTGTTCACTGGTGTCGTCCCTATTCTGGTGG
AACTGGATGGTGATGTCAACGGTCATAAGTTTTCCGTGCGTGGCGAGGG
TGAAGGTGACGCAACTAATGGTAAACTGACGCTGAAGTTCATCTGTACT
ACTGGTAAACTGCCGGTACCTTGGCCGACTCTGGTAACGACGCTGACTT
ATGGTGTTCAGTGCTTTGCTCGTTATCCGGACCATATGAAGCAGCATGA
CTTCTTCAAGTCCGCCATGCCGGAAGGCTATGTGCAGGAACGCACGATT
TCCTTTAAGGATGACGGCACGTACAAAACGCGTGCGGAAGTGAAATTTG
AAGGCGATACCCTGGTAAACCGCATTGAGCTGAAAGGCATTGACTTTAA
AGAAGACGGCAATATCCTGGGCCATAAGCTGGAATACAATTTTAACAGC
CACAATGTTTACATCACCGCCGATAAACAAAAAAATGGCATTAAAGCGA
ATTTTAAAATTCGCCACAACGTGGAGGATGGCAGCGTGCAGCTGGCTGA
TCACTACCAGCAAAACACTCCAATCGGTGATGGTCCTGTTCTGCTGCCA
GACAATCACTATCTGAGCACGCAAAGCGTTCTGTCTAAAGATCCGAACG
AGAAACGCGATCATATGGTTCTGCTGGAGTTCGTAACCGCAGCGGGCAT
CACGCATGGTATGGATGAACTGTACAAA

### Example 7

### Synthetic BMC Operons Derived from Diverse Natural BMC Genes

The spatial organization of enzymes in cells provides a means to regulate and accelerate metabolic pathways. To isolate and enhance specific pathways, bacteria have evolved proteinaceous organelles called bacterial microcompartments (BMCs) that sequester enzymatic steps from the cytoplasm.[1] By encapsulating enzymes and pathway intermediates within a selectively permeable shell, BMCs may increase reaction rates, retain volatile intermediates, protect the cytoplasm from toxic intermediates, and enable recycling of enzyme cofactors.[1-4] The incorporation of synthetic microcompartments in engineered biological systems could similarly increase flux through specific metabolic pathways and allow new strategies for control of information and metabolites in the cell.

The potential of synthetic BMCs has been recognized for various applications. Incorporation of new carbon-fixing BMCs (synthetic carboxysomes) into plants could increase biomass and carbon flux to biosynthetic pathways.[5, 6] Other synthetic microcompartments could be used to isolate engineered metabolic pathways from host cells to reduce toxicity and increase output.[7, 8] Encapsulation could increase shelf lives or facilitate catalyst removal in biocatalytic systems.[9] Microcompartments have additional potential application in therapeutics, including use as drug delivery vehicles and as scaffolds for vaccine development.[10] These diverse applications will require microcompartment systems that are robust to engineering experiments, are of precisely defined composition, and are amenable to detailed physical characterization.

To facilitate the design of new microcompartments, we sought to identify BMC shell systems that meet requirements for engineering: reliable purification of homogeneous compartments from genetically tractable organisms like E. coli, stability and amenability to mutation, and the capability of encapsulating new enzymes. While heterologous expression in E. coli has been reported for the three most well-characterized BMC systems, carboxysomes, propanediol utilization (pdu) compartments, and ethanolamine utilization (eut) compartments, substantial heterogeneity and low yields have been reported for these preparations.[11-13] Thus, we reasoned that screening the microcompartment shell proteins from the diversity of natural BMC proteins in sequenced genomes might enable discovery of shell systems more amenable to engineering and physical characterization.

We surveyed a diverse set of natural BMC gene clusters, many of unknown function, to identify new shell building blocks. We then designed a general strategy for producing synthetic BMC operons and found that one construct stood out in producing robust preparations of shells in high yield from expression in E. coli. This construct contained the seven BMC shell proteins found in the recently sequenced genome of the halophilic myxobacterium Haliangium ochraceum.[14] The homogeneity and small size of these shells enabled the construction of a structural model, suggesting roles for the individual shell proteins. New proteins could be targeted to these shells by fusion to peptide encapsulation tags, enabling design of new synthetic microcomparments. The tractability of this system should enable fundamental structural and mechanistic investigations of BMC function. Moreover, this system demonstrates the feasibility of producing self-assembling shells from diverse BMC building blocks selected from genomic sequence data without regard to native function or genomic context.

To identify BMC systems with properties needed for engineering, we selected a set of diverse, mostly uncharacterized natural BMC-associated genes and developed a general strategy for design of synthetic operons for heterologous expression of their shell genes in E. coli.

Natural BMC gene clusters vary widely in composition and gene arrangement, but all contain genes that encode shell proteins. Three types of BMC shell proteins exist, identified here as hexamers or BMC-H, tandem domains or BMC-T, and pentamers or BMC-P, that together form polyhedral shells (FIG. 1). BMC-H polypeptides contain a single domain of the pfam00936 family from the pfam database[15], about 90 amino acids, that assembles into a six-fold symmetric hexamer in available crystal structures.[16-18] This type of subunit represents the most abundant component of characterized BMC shells.[19-21] Tandem domains (BMC-T) contain two pfam00936 domains in a single polypeptide. These proteins form trimers with a pseudo-hexameric configuration that is sometimes found stacked into a double layer in crystal structures.[17, 18, 22, 23] The third and least abundant shell proteins, referred to here as pentamers or BMC-P, contain a single domain of the pfam03319 family. The five-fold symmetric assemblies formed by these units are presumed to occupy the vertices of icosahedral shells.[17, 18, 24]

Shell protein genes from eight BMC systems, many of unknown function, were selected for experimental testing (Table 1). These systems were chosen to sample from the diversity of BMCs in sequenced genomes in the number and types of shell proteins and the enzymes clustered with them. In several cases, the genes encoding shell proteins are found on multiple loci within the bacterial chromosome. The lack of a conserved number and order for shell protein genes form the various BMC gene clusters and the differences in transcription and translational initiation signals among organisms necessitated a generalized strategy for constructing synthetic BMC shell operons for testing.

TABLE 1
BMC clusters selected for testing.

| | Proposed BMC | Hexamers | Tandem | Pentamers |
|---|---|---|---|---|
| Organism | function | BMC-H | BMC-T | BMC-P |
| Alkaliphilus | Unknown | 3 | 0 | 1 |
| metalliredigens | | | | |
| QYMF | | | | |
| Clostridium | Unknown | 4 | 1 | 1 |
| phytofermentans | | | | |
| ISDg | | | | |
| Clostridium | Ethanolamine | 2 | 3 | 1 |
| phytofermentans | utilization | | | |
| ISDg | | | | |
| Leptotrichia buccalis | Ethanolamine | 4 | 2 | 1 |
| C-1013-b, DSM 1135 | utilization? | | | |
| Mycobacterium | Unknown | 1 | 2 | 1 |
| smegmatis MC2 155 | | | | |
| Planctomyces | Unknown | 2 | 0 | 3 |
| limnophilus Mu 290, | | | | |
| DSM 3776 | | | | |
| Rhodopseudomonas | Unknown | 3 | 2 | 1 |
| palustris BisB18 | | | | |
| Haliangium | Unknown | 1 | 3 | 3 |
| ochraceum SMP-2, | | | | |
| DSM 14365 | | | | |

The H. ochraceum shell proteins are found on three distinct chromosomal loci (FIG. 2A). The most extensive locus contains genes encoding four shell proteins (one BMC-H, two BMC-T, and one BMC-P) and a putative aldehyde dehydrogenase. A second locus encodes two BMC-P proteins and a putative aldolase. The amino acid sequences of both the aldehyde dehydrogenase and aldolase contain BMC encapsulation peptides at their N- and C-termini, respectively. These were predicted using the criteria of Kinney et al ((2012). Elucidating essential role of conserved carboxysomal protein CcmN reveals common feature of bacterial microcompartment assembly. Journal of Biological Chemistry 287, 17729-17736); encapsulation peptides are primary structure extensions at the N- or C-termini (relative to homologs found in genomes lacking BMC loci), the extension contains a short region (15-20 amino acids) that is predicted to form an alpha helix by typical secondary structure prediction tools (e.g. JPRED; Website compbio.dundee.ac.uk/www-jpred/). This helix is separated from the functional domain of the protein by a poorly conserved and often low complexity linker. The helical conformation of an encapsulation peptide has recently been confirmed by NMR solution structure analysis (Lawrence, A. D., Frank, S., Newnham, S., Lee, M. J., Brown, I. R., Xue, W-F., Rowe, M. L., Mulvihill, D. P., Prentice, M. B., Howard, M. J. and Warren, M. J. Solution structure of a bacterial microcompartment targeting peptide and its application in the construction of an ethanol bioreactor. ACS Synthetic Biology). Based on these criteria, the sequence of the predicted aldolase encapsulation peptide is RDDLVRVIREELVRALA (SEQ ID NO:52) while that of the aldehyde dehydrogenase is ALREDRIAEIVERVLARL (SEQ ID NO: 54). Moreover, the closest homologs to both H. ochraceum aldehyde dehydrogenase and aldolase also contain the extensions at their N- and C-termini and are found in other BMC gene clusters, strongly suggesting that they are involved in the metabolic function of the BMC. A third locus contains a gene encoding a BMC-T subunit; its adjacent genes do not resemble any other BMC-associated genes. Although the H. ochraceum shell protein genes are spatially separated, they are very likely associated with a single BMC. A scattered distribution of BMC genes encoding a single type of organelle is not uncommon; the beta-type carboxysome is a well-known precedent. The presence of an aldehyde dehydrogenase gene suggests that, like other catabolic BMCs, the H. ochraceum BMC sequesters an aldehyde intermediate. However, gene context does not provide further clues to the function of this uncharacterized BMC. Transmission electron microscopy of thin sections of H. ochraceum grown under standard cultivation conditions (DSM catalog 14365) show no obvious evidence of BMCs (see Supplemental Material). Expression of the organelle likely requires the presence of the substrate as observed for all catabolic BMCs characterized to-date (i.e. Eut, Pdu, and two distinct types of fucose catabolizing metabolosomes found in Clostridium phytofermentans and Planctomyces limnophilus). See Roof, D. M. a. R., J. R. (1988). Ethanolamine utilization in Salmonella typhimurium. Journal of Bacteriology 170, 3855-3863; Bobik, T. A., Ailon, M., Roth, J. R. (1992). A single regulatory gene integrates control of vitamin B12 synthesis and propanediol degradation. Journal of Bacteriology 174, 2253; Petit E, L. W., Coppi M V, Warnick T A, Currie D, Romashko I, Deshpande S, Haas K, Alvelo-Maurosa J G, Wardman C, Schnell D J, Leschine S B, Blanchard J L. (2013). Involvement of a Bacterial Microcompartment in the Metabolism of Fucose and Rhamnose by Clostridium phytofermentans. PLoS One 8:e54337; and Erbilgin, O., McDonald, K. L. and Kerfeld, C. A. Characterization of a Planctomycetal Organelle: A Novel Bacterial Microcompartment for the Aerobic Degradation of Plant Saccharides. Applied and Environmental Microbiology in press.

The fact that these genes are found on distinct chromosomal loci and the absence of information about transcriptional and translational levels of the H. ochraceum shell proteins necessitated the construction of a synthetic operon for expression of the H. ochraceum genes (FIG. 2B). We designed the operon to mimic shell protein ratios typical of natural BMCs that have been isolated from native organisms. Shell genes from H. ochraceum were synthesized with E. coli codon optimization in a single operon: the BMC-H subunit was placed first, followed by the genes for the BMC-T and then the BMC-P proteins. The BMC-H gene was preceded by a ribosomal binding site (RBS) with high predicted translation initiation rates, the BMC-T genes were preceded by intermediate RBS sequences, and BMC-P genes with low-level RBS sequences.

These gene constructs were expressed in E. coli under control of the T7 promoter. Because BMCs typically sediment upon centrifugation at ˜50,000×g,[19-21] the cell extracts were ultracentrifuged and screened for the presence of shell proteins in the pelleted fraction. From this screen, a construct built from the shell proteins of Haliangium ochraceum was identified as producing a distinct pattern of four proteins upon expression and ultracentrifugation, suggesting the presence of assembled structures (FIG. 2A). We discuss purification, characterization, and encapsulation experiments with the resulting shells in the following sections. In addition to shells identified from H. ochraceum, ultracentrifuge extracts revealed shell proteins in a few other cases (data not shown) and thin-section electron microscopy of E. coli cells expressing the synthetic operons showed a diverse range of cellular structures as has been previously observed upon expression of pdu and eut BMC shell genes in E. coli.[12, 13]

Purification of Microcompartment Shells.

Purification and electron microscopy experiments established that assembled microcompartment shells were produced upon heterologous expression of the H. ochraceum construct in E. coli. Ultracentrifuge extracts from expression of the H. ochraceum construct were fractionated by density gradient ultracentrifugation as well as by electrophoresis on 0.2% agarose[19], revealing in both cases a consistent pattern of four bands by SDS-PAGE (FIG. 2). Trypsin digests and mass spectrometry unambiguously identified these bands as the single BMC-H and three BMC-T proteins encoded by the synthetic H. ochraceum operon (FIG. 2B). The three BMC-T proteins are distinguished here with their gene locus tag numbers. The protein encoded by locus tag Hoch_{—}5812 is referred to as BMC-T(5812), for example. BMC-P proteins are expected to represent minor components of shells[17, 18, 24] and were not detected by mass spectrometry. Thus, from this construct, four major shell protein components are expected in assembled shells, and all four major components copurified in constant ratios.

Negatively stained electron microscopy with these purified fractions revealed distinct microcompartment shells (FIG. 3A). To allow further identification of the shells as products of heterologous expression, we specifically labeled the shell proteins. Upon immunogold labeling of these extracts with antibodies raised against H. ochraceum shell proteins, electron microscopy (TEM) indicated the association of gold particles with the shells (FIG. 3B). The expected icosahedral symmetry was supported by the appearance of facets in some TEM images, a nonrandom distribution of angles between gold particles, and the observation of triangular placement of gold particles (See SI Appendix).

We tested whether shells could be visualized in TEM images of sectioned E. coli cells. Shells were not readily evident in these images. The difficulty in viewing shells in sectioned cells may be due to the fact that the thickness of the TEM sections (40 nm) is similar to the shell diameter. Thus, cellular material is expected to contribute substantially to the TEM signal relative to the shell outline that is seen clearly with purified compartments in FIG. 3A. Because unambiguous visualization of the shells within E. coli cells was not obtained, there remains the possibility that the shells could assemble during the purification process rather than in vivo. Nevertheless, it is contemplated that the dark areas visible in the TEM images may contain a combination of densely packed shells and overexpressed shell proteins. Further experiments to label the shell proteins within cells may distinguish between these possible scenarios. Herein, we focus on characterizing the components and stoichiometry of the purified shells and we propose models for their construction.

The shells were remarkably homogeneous and could be isolated in high yield. Measured diameters of the shells from TEM images were 39±2 nm (79 shells measured). The particles were thus smaller and more homogeneous in size than those of previously isolated BMCs, including carboxysomes, pdu, or eut compartments, which have ranged in diameter from about 90-150 nm and have had standard deviations, where reported, of more than 20% of the diameter.[11-13, 20, 21, 25] Shell particle yields were also high. From protein concentrations, the quantity of shells was estimated at 10¹²-10¹¹ particles per L of culture. Consistent with this figure, a lower limit of 10¹¹-10¹⁰ particles per L of culture yield was estimated based on particle densities on electron microscopy grids. The shells appeared stable for two months or more at 4° C., and could be maintained in simple buffers or water.

Requirements for Shell Formation.

As a step toward determining the composition and construction of these shells, we sought to identify which of the seven Haliangium ochraceum genes were required for shell formation. Constructs were generated to systematically knockout individual shell genes either by complete removal or by mutagenesis to insert stop codons early in the gene. These constructs were tested for the presence of shell proteins in ultracentrifuge pellets and for the production of shells detectable by TEM.

Knockout of the single BMC-H gene led to loss of all shell proteins in ultracentrifuge extracts as well as loss of all detectable shells with TEM. This result was expected, as hexamers represent the major component of the shells. Although necessary, the BMC-H subunit alone was not sufficient to produce shells. When only the BMC-H subunit was expressed, shells were not detected in TEM studies of ultracentrifuge extracts. However, some BMC-H was detected in these extracts by SDS-PAGE, suggesting the possibility of higher-order structures or insolubility. Indeed, TEM images from pure BMC-H protein revealed extensive sheets of packed hexagons (FIG. 4A). Spacing of individual units in these images was estimated at 7.5 nm per hexamer, consistent with ˜70 Å diameter of BMC-H homologs that have crystallized in layers.[16-18] These two-dimensional assemblies are discussed further below.

In contrast to BMC-H, BMC-P subunits are expected to be minor components of icosahedral shells; only 60 monomers or 12 pentamers are required to close an icosahedral shell. The calculated molecular weights of the three BMC-P proteins (9-13 kD) overlap with that of the abundantly produced BMC-H subunit (10 kD), so these proteins cannot be distinguished by SDS-PAGE. Mass spectrometry of the BMC-H band from SDS-PAGE did not reveal the presence of any of the three BMC-P proteins. Thus, it remains unclear whether BMC-P subunits were present and undetectable by mass spectrometry or whether BMC-P proteins were not present at all in the purified shells. This is not without precedent; despite decades of research on purified carboxysomes, it was only with the availability of antibodies that BMC-P proteins were identified in Western Blots.[17, 18, 24] We were not able to produce antibodies to H. ochraceum BMC-P proteins in this work. No cross-reactivity was found with antibodies for the CsoS4B BMC-P protein from the Prochlorococcus marinus MED4 carboxysome, which has less than 30% sequence identity with any of the three H. ochraceum BMC-P proteins.

To determine whether or not the BMC-P genes were required in the synthetic operon for production of shells, we knocked out each individual BMC-P gene and also deleted all three BMC-P genes simultaneously from the operon. We found that any individual BMC-P gene could be knocked out, and further, that shells were formed even upon simultaneous deletion of all three BMC-P genes. Expression of a construct consisting only of BMC-H and three BMC-T proteins resulted in an SDS-PAGE banding pattern identical to that from the full seven-gene construct (FIG. 4B). Moreover, shells produced without the BMC-P genes were identical in diameter from those produced from the seven-gene construct (FIG. 4C). These results indicate that the BMC-P subunits are not required for formation of the shells. E. coli BL21 (DE3) cells also contain a gene of the pfam03319 family, with 36%, 36%, and 43% identity with the three pfam03319 proteins of H. ochraceum. It is possible that a small number of these natural E. coli proteins could also be incorporated into the shells. However, because these genes were not overexpressed and would not naturally be upregulated under the growth conditions, we strongly expect these proteins to be present in very small numbers. No E. coli shell proteins were detected by mass spectrometry.

This work is consistent with prior work that has indicated that carboxysomes are assembled in H. neapolitanus even upon deletion of all BMC-P genes.⁴⁰ BMC-P units may thus act in some systems more like caps to seal the vertices of the shells than crucial structural elements. In other systems, BMC-P proteins may have more essential structural roles, as a CcmL deletion resulted in elongation of the majority of beta carboxysomes formed⁴¹ and PduN was required for heterologous production of the Pdu compartment.²⁹ On the other hand, BMC shells have been reported to form in E. coli by the expression of a single BMC-H protein from the Eut system; these shells are therefore devoid of any BMC-P subunits²⁸.[26].

Our results do not allow us to distinguish between two different possible scenarios for BMC-P proteins expressed from this synthetic H. ochraceum operon system. The BMC-P proteins may be present in shells but not detectable and, at the same time, not required for shell formation. Alternatively, it is also possible that the BMC-P proteins are not expressed or are not present in the shells. Experiments to produce antibodies against BMC-P proteins and to increase expression levels of the BMC-P protein may be necessary to determine whether or not BMC-P proteins can be incorporated into shell vertices. Because BMC-P proteins are expected to seal the shell, thereby completing the diffusional barrier required for substrate concentration,⁴⁰ the production H. ochraceum-based synthetic shells for sequestering substrates around encapsulated enzymes will require the presence of BMC-P pentamers.

We next probed the roles of the distinct BMC-T subunits. As noted above, expression of the hexamer subunit alone did not produce detectable shells, suggesting a requirement for BMC-T subunits. FIG. 4D shows the results of knocking out BMC-T proteins by mutagenesis to place a single stop codon within in the first 10 amino acids. Removal of BMC-T(5812) led to a >10-fold reduction in yield of shells. In contrast, deletion of BMC-T(5816) led to little or no reduction in shell yield and deletion of BMC-T(3341) led to ˜2-fold reduction in shell yield. Shells could be detected by TEM in extracts from all three deletion mutants, although in substantially reduced numbers for the BMC-T(5812) deletion construct. The different effects of stop codons on shell yields among the three BMC-T units suggest that BMC-T(5812) may play a different or more crucial role, or that BMC-T(3341) and BMC-T(5816) may be functionally interchangeable. We note that, upon deletion of BMC-T(3341) or BMC-T(5816), the levels of the remaining BMC-T do not increase to the extent expected for full replacement of the missing BMC-T (FIG. 4D), indicating either that these two proteins may not be fully interchangeable or that their concentration in the cell is limiting. As discussed in the next section, sequence comparisons also reflect distinctions between BMC-T(5812) and the other two BMC-T units.

While the fact that shell yields were not reduced substantially upon knockout of BMC-T(5816) or BMC-T(3341) suggests possible redundancy of these two proteins, different results were obtained when the BMC-P subunits were deleted. A construct comprising only three genes, BMC-H, BMC-T(5812), and BMC-T(3341) (or, in other words, lacking both BMC-T(5816) and the BMC-P genes) was tested for the presence of shells in ultracentrifuge pull-down experiments. In these experiments, no shell proteins were visible by SDS-PAGE in the extracts from ultracentrifugation, indicating a dramatic reduction in shell yield. These results suggest that BMC-T(5816) could play a distinct role from BMC-T(3341) or that its presence could help to stabilize the icosahedron in the absence of pentameric caps.

Collectively, these results suggest that shell formation is facilitated by the energetically favorable formation of two-dimensional arrays from hexamers (FIG. 4A), and that tandem BMC domain proteins may be required to produce the curvature needed for complete shells even in the absence of BMC-P. We next evaluated the composition of shells more quantitatively with the goal of identifying specific roles for individual BMC proteins.

A Model for Shell Construction.

We used the available data to build a structural model for the shells. The remarkably consistent diameter measured in TEM experiments (39±2 nm, see above) combined with the dimensions of packed BMC-H hexamers (FIG. 4A), available crystal structures for shell protein homologs, and icosahedral construction placed strong constraints on possible geometries for the shell model. The resulting structural model comprised 12 pentagonal vertices and 260 hexagonal units, with 13 hexagons per face (FIG. 5C). In viral capsid nomenclature, this model corresponds to a triangulation number of T=27.[27]

We next used additional data to propose possible models for placement of individual shell proteins within the shell structure. The stoichiometry of individual shell proteins in purified shells was estimated by three methods. First, densitometry of SDS-PAGE gels with coomassie or silver staining gave mass ratios of 3:1:1:1 and molar ratios of 7:1:1:1 for monomeric units of the BMC-H and three BMC-T proteins, respectively. Because pentamers were not identified by mass spectrometry and no difference in stoichiometric ratio was detected between the 7- and 4-gene constructs, pentamers were not considered in determining stoichiometry. The molecular weights of the three BMC-T proteins are within 5% of each other and thus mass ratios and molar ratios of the BMC-T proteins are the same within error. Second, concentrations of individual purified shell proteins were determined by amino acid analysis and compared by titration on SDS-PAGE gels to purified shells to give molar ratios of 8:1:1:1. Third, amino acid analysis was performed directly on purified shells and the molar ratio was optimized to minimize the variation in particle concentration determined from each of the 14 individual amino acids that could be measured. This analysis also yielded a molar ratio of 8:1:1:1.

To place the shell protein stoichiometry within the context of the structural model, information about the biological assemblies of BMC-T and BMC-H subunits was required. BMC-T subunits have been shown to form double-layered hexameric assemblies as well as single-layered trimers in crystal structures.^{21; 43; 44; 45} Similarly, BMC-H proteins could form single-layered hexamers or, as recently proposed, double-layered dodecamers.[31] Thus, in calculating the number of hexagonal units in the icosahedral model, we considered possible scenarios in which both BMC-T and BMC-H subunits could be either single- or double-layered (FIG. 5A).

Combining these models with molar ratios of monomeric subunits of 8:1:1:1 and a structural model with 13 hexagons per face yields calculated values for the number of BMC-T or BMC-H hexagons in each face of the icosahedron (FIG. 5A, bottom). Models 2 and 3, in which both BMC-H and BMC-T are single- or double-stacked, yield a possible symmetric arrangement of hexagons, suggestive of different functional roles for the proteins. In these models, BMC-T pseudohexamers could serve the purpose of interacting with BMC-P pentamers to form five-fold vertices and producing curvature at intersections of the icosahedral faces. In contrast, Models 1 and 4 yield only asymmetric or random distributions of BMC-T and BMC-H.

To produce a more detailed model for shell construction, we considered sequence identities of the individual BMC-T subunits with available crystallographic structures. BMC-T(3341) and BMC-T(5816) have the greatest sequence identities (57% and 52%, respectively) with CcmP, a beta-type carboxysome BMC-T subunit that forms double-stacked layers in crystal structures.[22] These proteins contain conserved residues that line the trimer-trimer interface and are proposed to play a role in determining whether BMC-T units are single- or double-layered.[23] In contrast, BMC-T(5812) has the greatest sequence similarity (36%) with a PduT, a BMC-T unit from the pdu compartment that forms single-layered trimers.[28] These sequence similarities are consistent with the observations from BMC-T knock-out studies, suggesting that BMC-T(5812) may play a distinct role from BMC-T(5816) and BMC-T(3341). These differences are incorporated into Model 5 (FIG. 5B), which assigns the BMC-T subunits to distinct structural roles in shell construction. In this model, BMC-T(5812) forms a single layer that forms junctions between icosahedral faces while interacting with BMC-T(5812) and BMC-H units.

Model 5, although speculative, is consistent with the amino acid analysis data. Measurements of the thicknesses of shells from TEM images were consistent with an overall double-layered shell, although the limitations of this type of measurement do not allow us to reliably determine the thickness and a single-layered shell cannot be ruled out.

Immunogold labeling with antibodies raised against BMC-T(5812) showed triangular shapes, as seen in FIG. 3B, consistent with placement of these subunits at the vertices of the shells. Antibodies raised against BMC-T(3341) or the BMC-H subunit either showed no labeling or showed shell labeling without notable geometric patterns. Some TEM images showed thinning of the shell wall with angles that match those between the vertices of icosahedral structures, consistent with a reduced thickness from placement of single-layered BMC-T(5812) at the vertices (see SI Appendix). As noted above, deletion of BMC-T(5812) subunits substantially reduced the shell yield, supporting a distinct and non-redundant function for this type of subunit. Exact stoichiometries for Models 1-5 were all consistent with amino acid analysis data. Crystallographic structure determination and/or systematic perturbation of the individual shell proteins may provide further evidence to define the shell construction.

Together, the data support a shell structure composed of 260 hexagonal units and 13 hexagons per face. Based on stoichiometries of proteins in purified shells, we can propose five possible models for the placement of individual shell proteins. We emphasize that Models 1-5 are speculative and, although experimental evidence is consistent with these models, our data do not allow us to differentiate between them. We suggest that the energetically favorable self-association of BMC-H units to form layers (FIG. 4A) plays a key role in shell formation and that the different BMC-T proteins are required to stabilize the five-fold vertices and promote the curvature needed to form shells.

Targeting Proteins for Association with the Shell

The development of BMCs for compartmentalizing reactions or as polyvalent scaffolds will require the incorporation of enzymes that may not normally be expressed or associated with a BMC in the host organism. We tested whether a new protein (GFP) could be associated with the shells by fusion to either the aldehyde dehydrogenase (Hoch_{—}5813, FIG. 2A) of the native H. ochraceum BMC or its predicted encapsulation peptide. Encapsulation peptides have been recently predicted at the N- and C-termini of many BMC enzymes and are proposed to form alpha-helical structures that interact with shell proteins in order to incorporate the enzymes into the BMC.[32, 33] A gene encoding a putative aldehyde dehydrogenase that lies adjacent to BMC shell genes in the H. ochraceum genome (locus tag Hoch_{—}5813) is predicted to have an encapsulation peptide sequence at its N-terminus.[32]

Three constructs were co-expressed with the H. ochraceum shell proteins: GFP alone, GFP fused to the full-length sequence of the aldehyde dehydrogenase, and GFP fused to the first 34 residues at the N-terminus of this enzyme, the predicted encapsulation peptide (FIG. 6). A (GlySer)₁₀ linker was used in both fusions. Western blots of purified shells from these coexpressions indicated that GFP alone was not associated with the shells, whereas GFP fused to either the N-terminal region or the full enzyme was associated with the purified shells (FIG. 6). BMCs produced in the presence of these GFP constructs did not differ in size or morphology in TEM images from those produced without the GFP constructs. Both GFP fusions were found to copurify with shell proteins in sucrose density gradient ultracentrifugation experiments. Individual compartments were not visualized by fluorescence microscopy using the GFP label, as the cells glowed uniformly green, presumably due to excess GFP not associated with the compartments.

The ability to incorporate new proteins into the shells enables the design of new metabolic compartments as well as fundamental studies to understand the quantitative effects of enzyme encapsulation. The efficiency of association of the peptide tag complex with shells was lower than that of the full enzyme complex (FIG. 6). This observation suggests that either there are additional features of the full enzyme that help to recruit it to the shells or that the GFP-tag complex that we selected was not optimally suited for recruitment due to aggregation, suboptimal linker structure, or other factors.

Although the efficiency of targeting to the compartments was not high—western blots were needed to detect the associated GFP—these results do provide evidence that new proteins can be targeted for association with the compartments by fusion to encapsulation peptides or to enzymes associated with the native compartment. The simplest interpretation of these results is that these proteins are encapsulated within the lumen of the compartment as would be expected for the natural BMC enzyme. However, our experiments are not able to identify the location of the proteins, and it remains possible that the proteins are associated with the shells or the exterior of the compartments rather than encapsulated within the compartments. Indeed, the suggestion that some BMC enzymes associate with the external surface of the shell and “inject” metabolites into the compartment has recently been proposed for the Eut BMC (Huseby, D. L. & Roth, J. R. (2013). Evidence that a metabolic microcompartment contains and recycles private cofactor pools. Journal of Bacteriology 195, 2864). Other studies reporting encapsulation peptide-mediated targeting to BMCs were likewise unable to distinguish if the targeted protein localized to the lumen or was superficially associated with the shell (Sargent, F., Davidson, F. A., Kelly, C. L., Binny, R., Christodoulides, N., Gibson, D., Johansson, E., Kozyrska, K., Lado, L. L., MacCallum, J., Montague, R., Ortmann, B., Owen, R., Coulthurst, S. J., Dupuy, L., Prescott, A. R. and Palmer, T. (2013). A synthetic system for expression of components of a bacterial microcompartment. Microbiology 159, 2427-2436; Lawrence, A. D., Frank, S., Newnham, S., Lee, M. J., Brown, I. R., Xue, W-F., Rowe, M. L., Mulvihill, D. P., Prentice, M. B., Howard, M. J. and Warren, M. J. Solution structure of a bacterial microcompartment targeting peptide and its application in the construction of an ethanol bioreactor. ACS Synthetic Biology. Unequivocally identifying the location of heterologous cargo in synthetic shells is important for determining their potential applications. Future experiments will be needed to distinguish between these alternative possibilities; the results will dictate whether the H. ochraceum system is better suited for compartmentalization of enzymes or as a three-dimensional scaffold.

### Further Implications

Engineered BMCs as either scaffolds or compartments have the potential to increase product yields in metabolic engineering and to open up new avenues in biotechnology and synthetic biology. Further, understanding the self-assembly and function of BMCs will shed light on diverse biological processes including CO₂ fixation and bacterial pathogenicity in the mammalian intestine.[1, 34] Here we have described a new synthetic shell system based on the composition of a BMC that has only been characterized bioinformatically. This synthetic shell system offers essential advantages for both fundamental physical studies and engineering applications: a defined and homogeneous structural composition, reliable purification in high yield from E. coli, stability and robustness, and the potential for targeted association with new proteins.

The microcompartment shell system described here differs substantially from natural BMC particles that have been isolated previously, with a much smaller and more uniform size.[11-13, 20, 21, 25] The size and uniformity enabled the construction of a structural model that includes 260 hexagonal units total and 13 hexagonal units per icosahedral face (FIG. 5C). A further quantitative analysis of shell composition was used, in combination with sequence analysis, to suggest possible placements of the individual shell proteins within the shell structure. We propose that the BMC-H units readily self-assemble to form sheets as in FIG. 4A and that BMC-T units may serve to produce the curvature needed for shells and to produce the binding sites for pentameric vertex assemblies. Our model further suggests that two types of BMC-T units may have distinct roles in producing the icosahedral assembly. BMC-P units, in contrast, did not appear to be necessary for the formation of shells in this system, although they are expected to be a necessary component of the diffusive barrier needed for metabolic function.[26] The defined composition of these shells should enable systematic perturbations to further understand structural and functional roles of each unit.

This shell construct was produced synthetically, retaining no information about gene order, genome context, or potential biological function of the putative H. ochraceum microcompartment. The synthetic operon was designed to produce expression levels that mimic protein abundances in natural BMCs, the shell proteins were assembled into a new operon. It remains unknown whether the structure and properties of the shells are the same as those in the native H. ochraceum microcompartment in vivo, or whether an alternative type of shell is produced from this synthetic construct in E. coli. The native BMC has not been characterized and is not evident in TEM images of the organism grown under standard media conditions. Visualization of native BMCs by TEM typically requires induction of BMC production (Roof, D. M. a. R., J. R. (1988). Ethanolamine utilization in Salmonella typhimurium. Journal of Bacteriology 170, 3855-3863; Bobik, T. A., Ailon, M., Roth, J. R. (1992). A single regulatory gene integrates control of vitamin B12 synthesis and propanediol degradation. Journal of Bacteriology 174, 2253; Petit E, et al; Erbilgin, O., McDonald, K. L. and Kerfeld, C. A. Characterization of a Planctomycetal Organelle: A Novel Bacterial Microcompartment for the Aerobic Degradation of Plant Saccharides. Applied and Environmental Microbiology in press. (2013). Involvement of a Bacterial Microcompartment in the Metabolism of Fucose and Rhamnose by Clostridium phytofermentans. PLoS One 8:e54337.) for example by growing cells under conditions where the BMC-encapsulated metabolic pathway is required for growth. However, because the natural function of the H. ochraceum BMC is not known, isolation and characterization of the native BMC remains a challenge.

The goal of the present study was to devise methods for the production of synthetic shells to use as compartments or protein scaffolds for bioengineering, not to learn the function of the H. ochraceum BMC. In fact, this study is distinctive for its approach to synthetic shell production without regard to native function. The remarkable properties of these synthetic shells relative to results from the more familiar carboxysome, Pdu, and Eut systems illustrates the importance of sampling shell protein building blocks from the diversity of BMCs now apparent in sequenced genomes. The extent to which individual shell proteins can be swapped among different BMC types and source organisms remains unknown. But by heterologously coexpresing functionally related BMC shell proteins, it may be possible to produce other types of shells, even when the native functions are not known. Our results suggest that taking advantage of the many remaining BMC clusters of unknown function may offer new surprises and insights into BMC function and assembly

Engineered BMCs have the potential to increase product yields in metabolic engineering and to open up new avenues in biotechnology and synthetic biology. Further, understanding the self-assembly and function of BMCs will shed light on diverse biological processes including CO₂ fixation and bacterial pathogenicity in the mammalian intestine.[1, 34] Here we have described a new synthetic shell system that offers essential advantages for both fundamental physical studies and engineering applications: a defined and homogeneous structural composition, reliable purification in high yield from E. coli, stability and robustness, and the ability to encapsulate new proteins.

### Materials and Methods

Construction of Synthetic Operons.

For each natural BMC cluster, the shell proteins were categorized by type and placed in the following order after the promoter sequence: all BMC-H genes, all BMC-T genes, and finally all BMC-P genes. A ribosomal binding site sequence (RBS) was placed before each gene, such that expected translation initiation rates were greatest for BMC-H units and least for BMC-P units. Target predicted initiation rates had BMC-T and BMC-P genes at levels of about 50% and 5% of the predicted value for BMC-H, respectively. Gene synthesis was performed by Genscript using E. coli codon optimization.

Expression of Shell Proteins and Purification of Shells.

Synthetic operons were placed under the control of the T7 promoter in the pET-11 vector. Protein production in E. coli BL21-CodonPlus(DE3)-RIL was induced with 0.45 mM IPTG and cells were grown for 3-5 hours after induction at 37° C. Harvested cell pellets were suspended in (per 1 L growth): 2 mL TEMB buffer (5 mM Tris-Cl, 1 mM EDTA, 10 mM MgCl₂, 20 mM NaHCO₃, pH 8.0), 2 μL lysozyme (50 mg/mL), 10 μL DNAseI (2000 U/mL), 5 μL RNAse A (10 mg/mL). After a brief sonication (2×20 sec, 20% intensity, Branson micro tip), 2 mL BPER-II (Pierce) was added and the suspension was rocked at room temperature for 30 minutes. Cell debris was removed with an initial centrifugation at 12,000×g for 20 minutes. Extracts were then ultracentrifuged at 109,000 rcf (max) for 30 minutes. Small, glassy pellets were resuspended in TEMB. H. ochraceum shells were further purified with agarose gel electrophoresis as previously described[19] or with sucrose density gradient ultracentrifugation using step gradients of 20%-70% sucrose in TEMB with 5% step size.

Insertion of Stop Codons.

Inverse PCR mutagenesis was used to insert a single stop codon (TGA, TAA, or TAG) within the first 10 base triplets in each gene of the H. ochraceum construct. These knock-out mutations were expressed and purified as described for the standard construct.

Analysis and Quantitation of Shells and Shell Proteins.

Trypsin digests and mass spectrometry to identify H. ochraceum shell proteins from SDS-PAGE were performed by Stanford University Protein and Nucleic Acid Facility. Amino acid analysis of purified shell proteins and shells was performed by the U.C. Davis Genome Center Proteomics Core Facility. Deconvolution of shell protein amino acid analysis is further described. Estimates of protein mass ratios from gel densitometry were made using Image Lab software (Bio-Rad). Comparisons of protein masses from purified shells and between purified shells and quantitated shell proteins were made with multiple SDS-PAGE samples over 20-fold ranges of concentration, and uncertainties are estimated to be ˜20%.

Transmission Electron Microscopy and Gold Labeling.

Shells and shell proteins were negatively stained on formvar/carbon coated copper grids (Electron Microscopy Sciences, # FCF300-Cu) by floating the grids on a 4 μL sample droplet for 4 minutes, drying, and then floating the grid on a 4 μL droplet of 1% ammonium molybdate for 40 seconds. For immunogold labeling, antibodies were prepared by PRF & L (Canadensis, Pa.) using BMC-T(5812) protein purified by SDS-PAGE as the antigen. TEM Grids were floated on the BMC sample for 5 minutes, floated on 1% BSA with 0.05% Tween-20 in PBS buffer for 6 minutes to block, then floated at room temperature for 2 hours on primary antibody solution in a humid chamber. Grids were washed with 1% BSA in PBS and floated on 5 nm gold-labeled goat anti-rabbit (Abcam, # ab27235, 1 μg/mL in BSA/PBS) for 40 minutes then washed with PBS/BSA. After rinsing with water for 3×1 minutes, grids were stained by floating for 40 seconds on 1% uranyl acetate in water. Images were collected on Tecnai 12 and JEOL 1200EX TEM instruments.

Dynamic Light Scattering.

Shells were pelleted in a Ti-70 rotor at 42,000 rpm for 2 h and resuspended in 10 mM Tris pH 7.4. Shells at an A280 of 0.6 were measured in a Protein Solutions Dynapro dynamic light scattering instrument with an acquisition time of 5 s, averaging 10 acquisitions at a constant temperature of 25 C. The shell radius was calculated assuming a globular particle shape.

Encapsulation Experiments.

The constructs indicated in FIG. 6 were placed under control of the T7 promoter in the pCOLA-DUET-1 vector for coexpression with shell proteins in pET-11. Protein expression and shell purification was performed as described for shells. Western blots were performed with 1:3000 dilution of anti-GFP polyclonal antibody (Life Technologies) and detected with AP-conjugated anti-rabbit (Sigma) and BCIP/NBT substrate.

Our model suggests that two types of BMC-T units may have distinct roles in producing the icosahedral assembly. BMC-P units, in contrast, did not appear to be necessary for the formation of shells in this system, although they are expected to be a necessary component of the diffusive barrier needed for metabolic function.[26] The defined composition of these shells should enable systematic perturbations to further understand structural and functional roles of each unit.

This shell construct was produced synthetically, retaining no information about gene order, genome context, or potential biological function of the putative H. ochraceum microcompartment. Using a logic designed to produce expression levels that mimic protein abundances in natural BMCs, the shell proteins were assembled into a new operon. This strategy made it possible to consider a diverse set of potential new shell building blocks. It remains unknown whether the structure and properties of the compartments are recapitulated in a native H. ochraceum microcompartment in vivo. Nevertheless, the remarkable properties of these synthetic shells relative to results from the more familiar carboxysome, pdu, and eut systems illustrates the importance of sampling from the diversity of BMCs now apparent in sequenced genomes. Many of the BMC systems included in our screen were of unknown function and BMCs could be identified only by the presence of genes encoding shell proteins. Our results expand the range of known BMC shell morphologies and suggest that the many remaining BMC clusters of unknown function may offer new surprises and insights into BMC function and assembly

### Example 8

### Targeting Proteins for Encapsulation for Biofuel Production

Synthetic microcompartment shells containing Rubisco could be expressed in the chloroplast of tobacco or camelina to increase photosynthetic efficiency for agricultural or biofuel production.

Alternatively, the microcompartment shells could be designed to house enzymes for the production of high energy molecules (CoA derivative) or chemical feedstocks (isoprene) and expressed in plants to produce large quantities of desired product in crop or mass cultured species

### Example 9

### Using Self-Assembling Microcompartment Proteins to Form Sheets

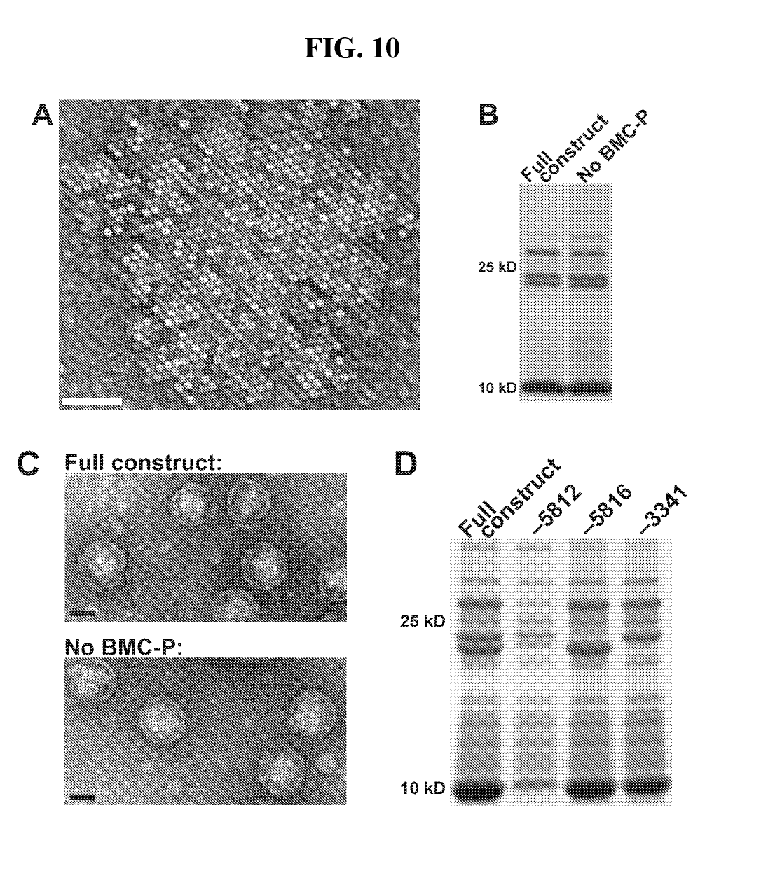

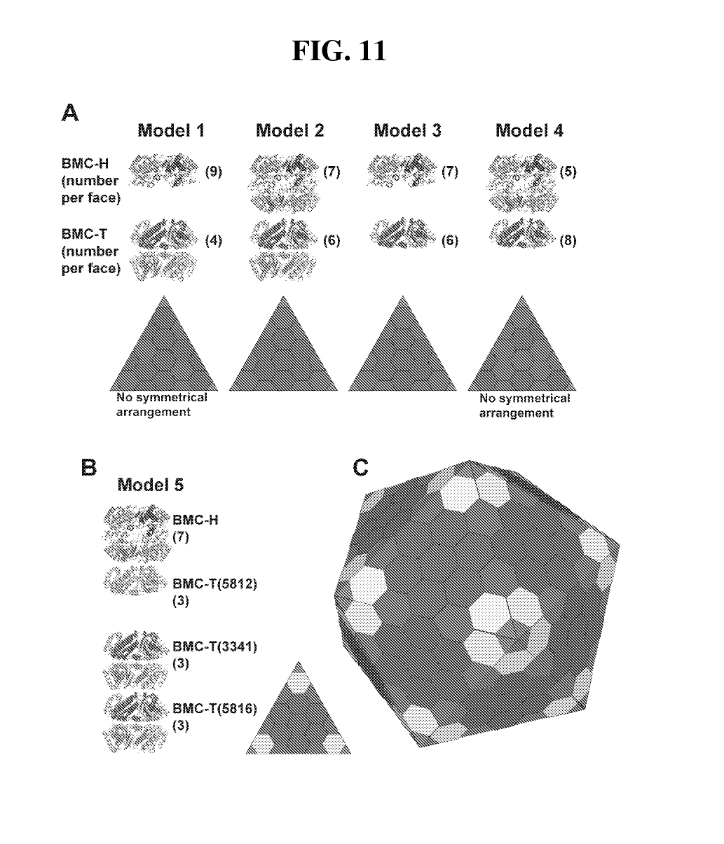

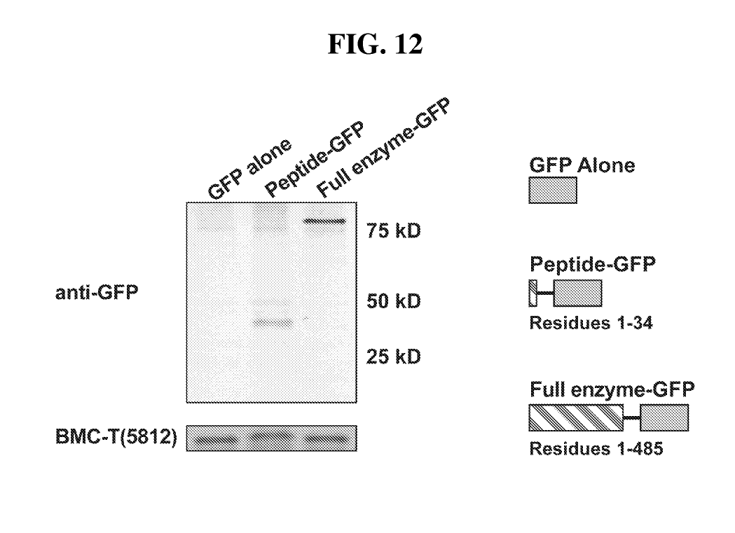

BMC shell proteins may also be used to produce self-assembling sheets that may be used for a variety of applications, including fabrication, production of molecular sieves, or creation of an ordered system with properties similar to those of liquid crystal. We observed self-assembly into protein layers of shell proteins from H. ochraceum. The hexamer protein (single pfam00936 domain) from H. ochraceum was expressed in E. coli and purified in a single extraction step with the BPER-2 reagent (Pierce). TEM images of this pure sample on Formvar carbon grids showed large sheets of closely-packed hexagonal proteins as shown in FIG. 10A

The microcompartment shell system described here differs substantially from natural BMC particles that have been isolated previously, with a much smaller and more uniform size.[11-13, 20, 21, 25] The size and uniformity enabled the construction of a structural model that includes 260 hexagonal units total and 13 hexagonal units per icosahedral face (FIG. 5C). A further quantitative analysis of shell composition led to a model for shell construction that suggests specific structural roles for the four gene products. Our results suggest that the BMC-H units readily self-assemble to form sheets as in FIG. 4A and that BMC-T units may serve to produce the curvature needed for shells and to produce the binding sites for pentameric vertex assemblies.

It may be possible to control the assembly into layers by altering the conditions. At this stage, however, it is known is that the layers form spontaneously under the given conditions as described in Example 7. It appears that in solution, the layers are constantly forming and dissociating because the pure protein solution has an iridescent and swirling appearance that is reminiscent of liquid crystal. Future experiments may be carried out to test or optimize what the requirements are in terms of protein concentrations, buffer conditions, temperature, etc.

### Example 10

### Evidence of Icosahedral Construction

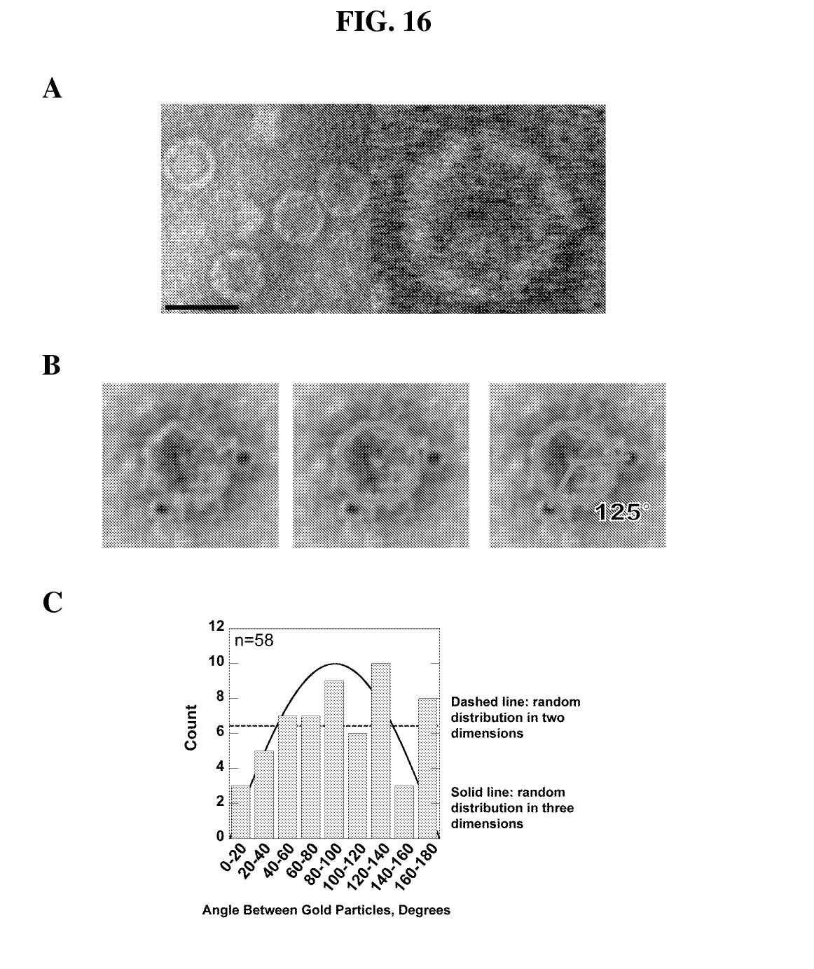

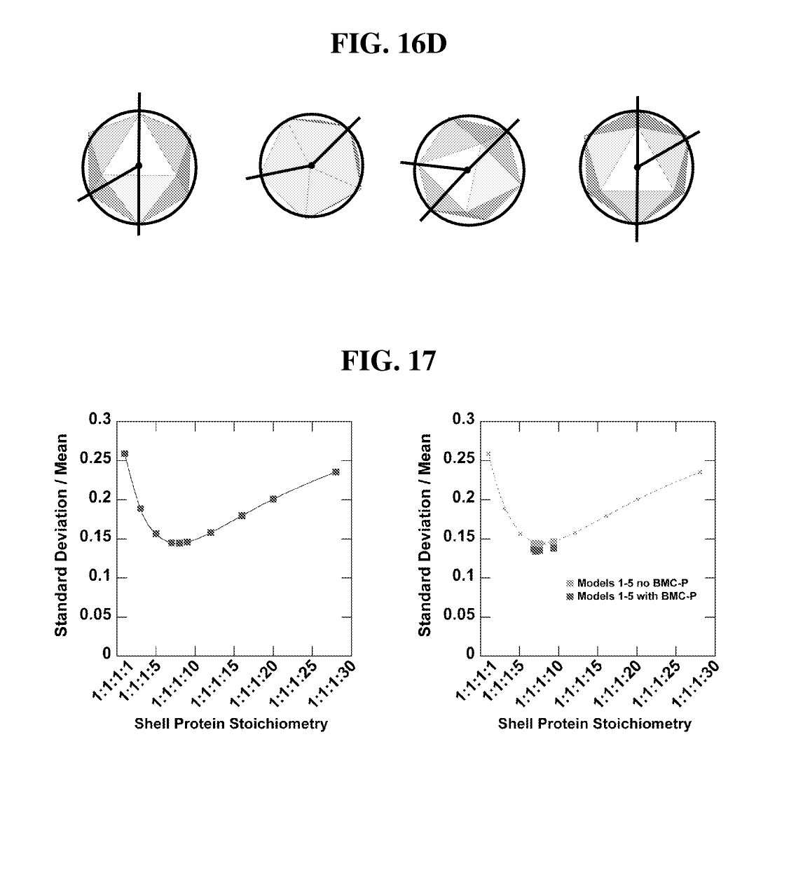

We also tested whether or not the angles between gold particles in TEM images reflect possible icosahedral symmetry. Angles were measured in images of shells that were treated with gold-labeled BMC-T(5812) antibodies. The angles between gold particles were determined as follows: Gold particles were selected only if the gold was positioned outside of the shell outline in TEM images. As shown in the example of FIG. 16B, a circle was drawn around the shell outline and used to define a center point. Angles were defined as gold-center-gold and all possible angles between gold particles were measured. These angles (n=58) are plotted in the histogram. In FIG. 16C, the expectations for two types of random distributions of angles are shown on the histogram. We have illustrated two limiting cases that represent random distributions of points placed either on the circumference of a two-dimensional circle or on the surface of a three-dimensional sphere.

A dashed line is shown that corresponds to the histogram expected for 58 measurements of angles between hypothetical points that are randomly positioned on a two-dimensional circle. These hypothetical points on a circle represent the expectations for a random distribution of angles in the case where our gold particles were perfectly selected such that the antibody binding sites were positioned exactly on the outermost edge of the shells. If the gold particles were perfectly selected in this way, they would correspond to points on a circle, and the random distribution is expected to be flat, as seen in the dashed line.

However, the selection of gold particles is likely to be imperfect because of the ˜7 nm length between the antibody binding site and the difficulty in knowing the site of binding of the antibody on the shell. The resulting imperfect measurements are expected to be intermediate between the above two-dimensional model and the distribution of angles between gold particles positioned on a sphere that is projected into two dimensions. The solid line illustrates the shape of the histogram expected for 58 measurements of angles between particles positioned on a three-dimensional sphere that is projected into a two-dimensional image. This distribution is curved, because the surface area of the sphere at an angle of 90° is much larger than the surface area of the sphere at an angle near 180°. As an example of this phenomenon, there are many different ways to stand at the equator of the earth (90°) but only one way to stand on the north pole (180°).

In contrast to both types of random distributions, the observed distribution of angles, shown in the histogram bars, suggests a higher representation of 120°-140° and 160°-180° angles, as would be expected for vertices of icosahedral particles projected onto two dimensions. Examples of these angles that are expected to be more highly represented in icosahedral particles are shown in FIG. 16D.

### Example 11

### Stoichiometry of Shell Proteins from Amino Acid Analysis Performed on Purified Shells

Amino acid analysis was performed on shells purified by multiple passages over sucrose gradients. The resulting shell preparation was similar to (but more pure than) that shown in FIG. 2C in the manuscript under the heading “Sucrose purification”. Purity is estimated at >90%, and the concentration of any single impurity was low.

Amino acid analysis of these shells yielded values for concentrations of 14 amino acid groups: Asp+Asn, Gln+Glu, Thr, Ser, Pro, Gly, Ala, Val, Ile, Leu, Tyr, Phe, His, Lys, and Arg. Data for Lys were not considered further because they showed large deviations in concentration values relative to the other amino acids in tests with the individual purified shell proteins.

Values for the 14 amino acid groups were used to calculate concentrations of shell units as in the example in the table below. This example does not include BMC-P, but these units are included in analyses described on the next page.

The first column of values in the table corresponds to the experimentally determined molar concentration of the amino acid group in the sample of purified shells. These were calculated directly from the results of amino acid analysis. The next four columns (“Number of each amino acid in sequence”) correspond to the count of each amino acid in the primary sequence of the given polypeptide. These values were used to calculate the number of each amino acid that would be expected in a shell unit of 8:1:1:1 molar ratio. For example, 87 Asn+Asp residues were calculated for an 8:1:1:1 shell unit by adding (8×6 in BMC-H)+(1×1 in BMC-T(5816))+(1×1 in BMC-T(3341))+(1×1 in BMC-T(5812)).

In the right-most column of the table, the effective concentration of shell units is calculated by using the concentration of amino acid from column 1 and the number of amino acids in an 8:1:1:1 shell unit. For example, the 83.2 μM concentration of Asn/Asp residues was divided by the expected 87 Asn/Asp residues in a single 8:1:1:1 shell unit to give a concentration of shell units in the sample of 0.957. This column thus gives a calculated concentration of shell units obtained from the data for each of the 14 amino acid groups.

TABLE 2

| | | Number of | | | | | |
|---|---|---|---|---|---|---|---|
| | Concentration | each amino acid in sequence | | | | Number per shell | Concentration of |
| | of amino acid | | BMC-T | BMC-T | BMC-T | unit using 8:1:1:1 | shell units using |
| | in shells, μM | BMC-H | (5816) | (3341) | (5812) | molar ratio | 8:1:1:1 molar ratio |
| Asn/Asp | 83.2 | 6 | 12 | 14 | 13 | 87 | 0.957 |
| Thr | 52.0 | 4 | 11 | 8 | 9 | 60 | 0.867 |
| Ser | 34.2 | 1 | 9 | 6 | 9 | 32 | 1.069 |
| Gln/Glu | 118.8 | 8 | 30 | 29 | 22 | 145 | 0.820 |
| Pro | 59.4 | 4 | 9 | 7 | 11 | 59 | 1.007 |
| Gly | 137.2 | 12 | 15 | 21 | 13 | 145 | 0.947 |
| Ala | 209.3 | 19 | 33 | 35 | 36 | 256 | 0.817 |
| Val | 143.2 | 17 | 18 | 13 | 16 | 183 | 0.783 |
| Ile | 41.6 | 3 | 15 | 12 | 10 | 61 | 0.682 |
| Leu | 72.8 | 4 | 20 | 23 | 25 | 100 | 0.728 |
| Tyr | 19.4 | 2 | 6 | 4 | 1 | 27 | 0.719 |
| Phe | 18.2 | 1 | 7 | 4 | 6 | 25 | 0.728 |
| His | 23.2 | 2 | 4 | 5 | 3 | 28 | 0.829 |
| Arg | 88.8 | 6 | 12 | 13 | 16 | 89 | 0.998 |

We have an expectation that if the molar ratio of 8:1:1:1 is accurate, then the calculated molarity of shell units (the right-most column) should be similar among each of the 14 different amino acid measurements. Similarly, if the molar ratio is far off from the true value, then different amino acids will be differentially represented and the calculated molarity of shell units will vary more substantially among the amino acids. While some amino acids are similarly represented among BMC-H and the BMC-T proteins, other amino acids such as Gln/Glu and Leu, have 2-fold to 6-fold differences in abundances and these variations will significantly affect the calculated molarities leading to wider variation among the amino acids if the molar ratio is inaccurate

We thus performed the calculations for a wide range of possible molar ratios, ranging from 1:1:1:1 to 30:1:1:1. To determine the degree of variation in calculated molarities of shell units among the different amino acids, we calculated standard deviations for the values in the right-most column in the table. The standard deviations divided by the means are plotted in red below for molar ratios ranging from 1:1:1:1 to 30:1:1:1. In these plots, the smallest variation was found with a molar ratio of 8:1:1:1, consistent with data from gel densitometry as described herein.

The data plotted in FIG. 17 left panel are those calculated with simple ratios of shell units as given in the example table on the prior page. We also plotted (FIG. 17 right panel) the results when exact stoichiometries for models 1-5 in the main text are used in the calculations, both with and without BMC-P subunits. Values in green include BMC-P, while values in orange do not include BMC-P. The variation is slightly lower when BMC-P subunits are included, but the differences are small and cannot rule out either the presence or absence of pentameric subunits.

### Example 12

### Measurement of Thickness of Shells in TEM Images

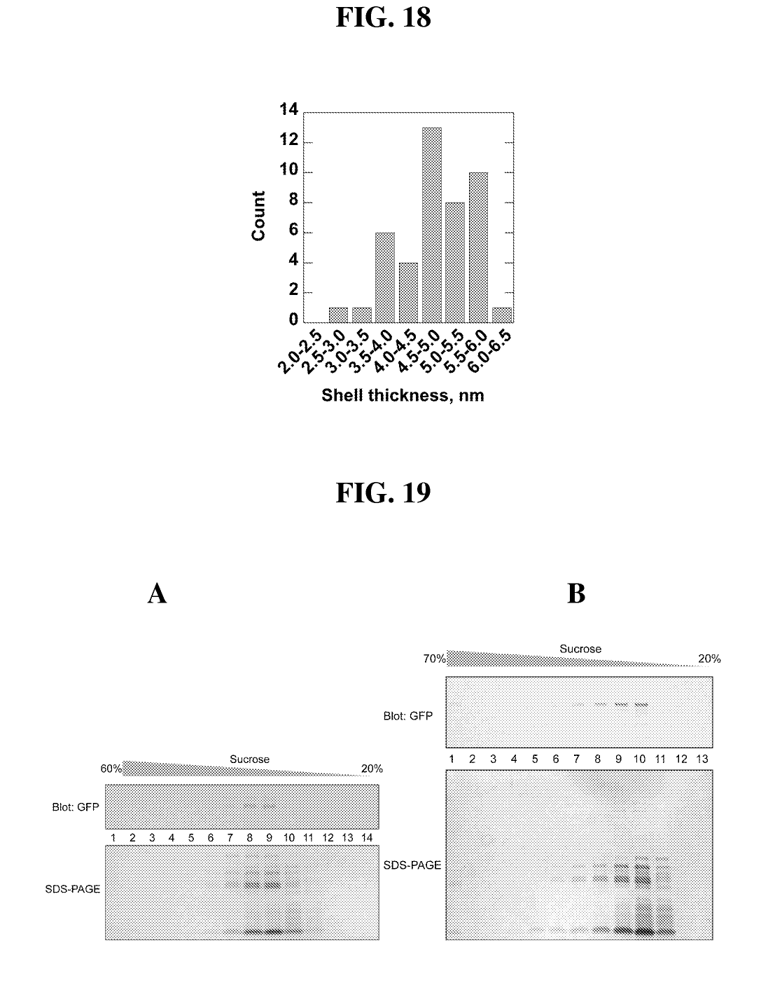

Shell thicknesses were measured in TEM images that were collected as described in Materials and Methods. Measurements from 31 different shells were taken from TEM images collected on two different instruments and from two different shell preparations. The distribution of thickness values is shown in FIG. 18.

The distribution of values suggests that the shell thicknesses are greater than those expected for a single-layered shell. Measurements from crystal structures of BMC-H homologs give a range of about 2.9-3.3 nm (e.g., pdb code 2EWH). However, there is substantial uncertainty in measuring these values from TEM images, including concerns about resolution, staining, and the effects of the drying process.

### Example 13

### Copurification of GFP-Labeled Constructs with Microcompartment Shells

Following co-expression of shell proteins (in the pET-11 vector) with the GFP fusions shown in FIG. 4 (in the pCOLA-DUET-1 vector) in E. coli BL21-CodonPlus(DE3)-RIL cells, the purification procedure described in Materials and Methods was followed. Following ultracentrifugation, the supernatants were separated with sucrose step gradients (20%-70% sucrose in TEMB buffer with 5% step sizes). BMC-containing fractions were pooled and concentrated by ultracentrifugation, then placed over a second sucrose gradient. Shown in FIG. 19 are SDS-PAGE and western blotting of the fractions collected from this purification step.

### REFERENCES

1. Kerfeld, C. A., S. Heinhorst, and G. C. Cannon, Bacterial Microcompartments. Annual Review of Microbiology, 2010. 64: p. 391-408.
2. Cheng, S., et al., The PduQ enzyme is an alcohol dehydrogenase used to recycle NAD(+) internally within the Pdu microcompartment of salmonella enterica. PLoS One, 2012. 7: p. e47144.
3. Huseby, D. L. and J. R. Roth, Evidence that a metabolic microcompartment contains and recycles private cofactor pools. Journal of Bacteriology, 2013.
4. Dou, Z., et al., CO2 fixation kinetics of Halothiobacilius neapolitanus mutant carboxysomes lacking carbonic anhydrase suggest the shell acts as a diffusional barrier for CO2. Journal of Biological Chemistry, 2008. 283: p. 10377-10384.
5. Price, G. D., et al., The cyanobacterial CCM as a source of genes for improving photosynthetic CO2 fixation in crop species. Journal of Experimental Botany, 2013. 64: p. 753-768.
6. Zarzycki, J., et al., Cyanobacterial-based approaches to improving photosynthesis in plants. Journal of Experimental Botany, 2013. 64: p. 787-798.
7. Agapakis, C. M., P. M. Boyle, and P. A. Silver, Natural strategies for the spatial optimization of metabolism in synthetic biology. Nature Chemical Biology, 2012. 8: p. 527-535.
8. Chen, A. H. and P. A. Silver, Designing biological compartmentalization. Trends in Cell Biology, 2012. 22: p. 662-670.
9. Howorka, S., Rationally engineering natural protein assemblies in nanobiotechnology. Current Opinion in Biotechnology, 2011. 22: p. 485-491.
10. Doll, T. A. P. F., et al., Nanoscale assemblies and their biomedical applications. Journal of the Royal Society Interface, 2013. 10: p. 20120740.
11. Bonacci, W., et al., Modularity of a carbon-fixing protein organelle. Proceedings of the National Academy of Sciences of the United States of America, 2012. 109: p. 478-483.
12. Choudhary, S., et al., Engineered protein nano-compartments for targeted enzyme localization. PLoS One, 2012. 7: p. e33342.
13. Parsons, J. B., et al., Synthesis of empty bacterial microcompartments, directed organelle protein incorporation, and evidence of filament-associated organelle movement. Molecular Cell, 2010. 38: p. 305-315.
14. Ivanova, N., et al., Complete genome sequence of Haliangium ochraceum type strain (SMP-2). Standards in Genomic Sciences, 2010. 2: p. 96-106.
15. Punta, M., et al., The Pfam protein families database. Nucleic Acids Research, 2012. 40: p. D290-D301.
16. Kerfeld, C. A., et al., Protein structures forming the shell of primitive bacterial organelles. Science, 2005. 309: p. 936-938.
17. Kinney, J. N., S. D. Axen, and C. A. Kerfeld, Comparative analysis of carboxysome shell proteins. Photosynthetic Research, 2011. 109: p. 21-32.
18. Yeates, T. O., C. S. Crowley, and S. Tanaka, Bacterial microcompartment organelles: Protein shell structure and evolution. Annual Review of Biophysics 2010. 39: p. 185-205.
19. Cannon, G. C. and J. M. Shively, Characterization of a homogeneous preparation of carboxysomes from Thiobacillus neapolitanus. Archives of Microbiology, 1983. 134: p. 52-59.
20. Havemann, G. D. and T. A. Bobik, Protein content of polyhedral organelles involved in coenzyme B12-dependent degradation of 1,2-propanediol in Salmonella enterica serovar typhimurium LT2. Journal of Bacteriology, 2003. 185: p. 5086-5095.
21. Roberts, E. W., et al., Isolation and characterization of the prochlorococcus carboxysome reveal the presence of the novel shell protein CsoS1D. Journal of Bacteriology, 2012. 194: p. 787-795.
22. Klein, M. G., et al., Identification and structural analysis of a novel carboxysome shell protein with implications for metabolite transport. Journal of Molecular Biology, 2009. 392: p. 319-333.
23. Cai, F., et al., The structure of CcmP, a tandem bacterial microcompartment domain protein from the β-carboxysome forms a subcompartment within a microcompartment. 2013. 288: p. 16055-16063.
24. Tanaka, S., et al., Atomic-level models of the bacterial carboxysome shell. Science, 2008. 319: p. 1083-1086.
25. So, A. K. C., et al., A novel evolutionary lineage of carbonic anhydrase (ε class) is a component of the carboxysome shell. Journal of Bacteriology, 2004. 186: p. 623-630.
26. Cai, F., et al., The pentameric vertex proteins are necessary for the icosahedral carboxysome shell to function as a CO2 leakage barrier. PLoS One, 2009. 4: p. e7521.
27. Caspar, D. L. D. and A. Klug, Physical principles in the construction of regular viruses. Cold Spring Harbor Symposia on Quantitative Biology, 1962. 27: p. 1-24.
28. Pang, A., M. J. Warren, and R. W. Pickersgill, Structure of PduT, a trimeric bacterial microcompartment protein with a 4Fe-4S cluster-binding site. Acta Crystallographica, Section D, 2011. 67: p. 91-96.
29. Crowley, C. S., et al., PduT C38S mutant from Salmonella enterica Typhimurium. Journal of Biological Chemistry, 2010. 285: p. 37838-37846.
30. Sagermann, M., A. Ohtaki, and K. Nikolakakis, Crystal structure of the EutL shell protein of the ethanolamine ammonia lyase microcompartment. Proceedings of the National Academy of Sciences of the United States of America, 2009. 106: p. 8883-8887.
31. Samborska, B. and M. S. Kimber, A dodecameric CcmK2 structure suggests β-carboxysomal shell facets have a double-layered organization. Structure, 2012. 20: p. 1353-1362.
32. Kinney, J. N., et al., Elucidating essential role of conserved carboxysomal protein CcmN reveals common feature of bacterial microcompartment assembly. Journal of Biological Chemistry, 2012. 287: p. 17729-17736.
33. Fan, C., et al., Short N-terminal sequences package proteins into bacterial microcompartments. Proceedings of the National Academy of Sciences of the United States of America, 2010. 107: p. 7509-7514.
34. Thiennimitr, P., et al., Intestinal inflammation allows Salmonella to use ethanolamine to compete with the microbiota. Proceedings of the National Academy of Sciences of the United States of America, 2011. 108: p. 17480-17485.

The above examples are provided to illustrate the invention but not to limit its scope. Other variants of the invention will be readily apparent to one of ordinary skill in the art and are encompassed by the appended claims. All publications, references, databases, and patents cited herein are hereby incorporated by reference for all purposes.

What is claimed is:

1. A method for producing bacterial microcompartments in a host organism, said method comprising introducing into an organism an expression vector comprising a heterologous nucleotide sequence or a fragment thereof comprising (a) an operably linked promoter that drives expression in the organism; (b) a ribosomal binding site sequence that controls expression efficiency in the organism, and (c) a bacterial microcompartment gene or designed variant thereof derived from a bacteria for producing a bacterial microcompartment.

2. The method of claim 1, wherein the host organism is a prokaryotic cell or a eukaryotic cell.

3. The method of claim 2, wherein the host organism is E. coli, B. subtilis, S. cerevisiae. cyanobacteria, plants, or algae.

4. An expression vector comprising a polynucleotide comprising one or more hexamer genes, tandem domains, and pentamers, constructed with a gene order and a set of RBS sequences preceding each gene such that the expression levels are highest for hexamers, intermediate for tandem domains, and lowest for pentamers.

5. A cell comprising in its genome at least one stably incorporated expression cassette, said expression cassette comprising a heterologous nucleotide sequence of claim 4 operably linked to a promoter that drives expression in the cell.

6. The cell of claim 5, wherein the cell is a prokaryotic cell or a eukaryotic cell.

7. The cell of claim 6, wherein the cell is E. coli, B. subtilis, S. cerevisiae. cyanobacteria, plant, or algae.

8. A plant comprising in its genome at least one stably incorporated expression cassette, said expression cassette comprising a heterologous nucleotide sequence of claim 4 operably linked to a promoter that drives expression in the plant.

9. The plant of claim 6, wherein the plant is Tobacco

10. A composition comprising an isolated empty bacterial microcompartment.

11. The composition of claim 10 wherein the isolated microcompartment is constructed from shell proteins derived from Haliangium ochraceum proteins with accession numbers YP_{—}003270184 (SEQ ID NO:6), YP_{—}003270181 (SEQ ID NO:8), YP_{—}003267736 (SEQ ID NO:10), YP_{—}003270185 (SEQ ID NO:12), and variants thereof.

12. The composition of claim 10 wherein the isolated microcompartment is constructed from shell proteins derived from Thermosynechococcus elongatus proteins SEQ ID NO: 40, SEQ ID NO:41, SEQ ID NO:42, SEQ ID NO:43, SEQ ID NO:44, and SEQ ID NO:45 and variants thereof.

13. The compositions of claim 10 further comprising a label, tag or other molecule coexpressed and/or fused to the microcompartment.

14. A method for enhancing metabolic activity in an organism, said method comprising introducing into the organism an expression cassette comprising a heterologous nucleotide sequence or a fragment thereof comprising (a) an operably linked promoter that drives expression in the organism; (b) a ribosomal binding site sequence that controls expression efficiency in the organism, and (c) bacterial microcompartment gene derived from a bacteria for producing a bacterial microcompartment, and further providing a polynucleotide encoding an encapsulating targeting peptide or a fragment thereof linked to a polynucleotide encoding a protein that provides enhanced metabolic activity in an organism.

15. The method of claim 6 wherein the polynucleotide expressing an encapsulating targeting peptide is native to the bacteria that the bacterial compartment genes are derived from.

16. The method of claim 6 wherein the polynucleotide expressing an encapsulating targeting peptide comprising a sequence selected from SEQ ID NOS: 24 and 28.

17. A composition comprising self-assembled protein sheets comprising two-dimensional ordered hexamer shell proteins of Haliangium ochraceum proteins with accession numbers YP_{—}003270184 (SEQ ID NO:6), YP_{—}003270181 (SEQ ID NO:8), YP_{—}003267736 (SEQ ID NO:10), YP_{—}003270185 (SEQ ID NO:12), and variants thereof.


SEQUENCE LISTING (58 sequences)

SEQ ID NO: 1 - DNA, 25 bp, Escherichia coli
tttagagaaagaggagaaatactag

SEQ ID NO: 2 - DNA, 28 bp, Escherichia coli
tttagagattaaagaggagaaatactag

SEQ ID NO: 3 - DNA, 27 bp, Escherichia coli
tttagagtcacacaggaaacctactag

SEQ ID NO: 4 - DNA, 34 bp, Halothiobacillus neapolitanus
gattttgaatgagtctttattgaggagagaagaa

SEQ ID NO: 5 - DNA, 255 bp, Verminephrobacter eiseniae
atgactgcatacatcaatcaggaagcagcggcaaagctcgatgaagtctttccccttgcagaattggcgctcaaggcctatgggccagagtttcagggtgaactcagcctgctgacacactctgaaaattcgacctatctggtgaacgcattttcagggcagcgcttcgtaatgcgcgtgcatcgtgcgcactaccacagcagaacggccattgaaagcgagttggcatggctggatgcactggccgatgaaggc

SEQ ID NO: 6 - protein, 99 aa, Haliangium ochraceum
MADALGMIEVRGFVGMVEAADAMVKAAKVELIGYEKTGGGYVTAVVRGDVAAVKAATEAGQRAAERVGEVVAVHVIPRPHVNVDAALPLGRTPGMDKSA

SEQ ID NO: 7 - DNA, 300 bp, Artificial Sequence
  Synthetic DNA sequence selected to encode the protein sequence of SEQ ID NO6 while utilizing high-frequency codons from E. coli, the host organism; YP_003270184
atggcggacgcactgggtatgattgaagttcgtggttttgttggtatggtggaagcggcggatgctatggtgaaagcggctaaagttgaactgattggttatgaaaaaaccggcggtggctacgtgacggcagtggttcgtggtgatgtcgcagcagttaaggcagctaccgaagccggtcagcgtgcagcagaacgtgttggtgaagtcgtggcagttcatgtcatcccgcgtccgcacgtgaacgttgatgcagctctgccgctgggtcgtacgccgggtatggacaaaagcgcgtaa

SEQ ID NO: 8 - protein, 205 aa, Haliangium ochraceum
MDHAPERFDATPPAGEPDRPALGVLELTSIARGITVADAALKRAPSLLLMSRPVSSGKHLLMMRGQVAEVEESMIAAREIAGAGSGALLDELELPYAHEQLWRFLDAPVVADAWEEDTESVIIVETATVCAAIDSADAALKTAPVVLRDMRLAIGIAGKAFFTLTGELADVEAAAEVVRERCGARLLELACIARPVDELRGRLFF

SEQ ID NO: 9 - DNA, 618 bp, Artificial Sequence
  Synthetic DNA sequence selected to encode the protein sequence of SEQ ID NO8 while utilizing high-frequency codons from E. coli, the host organism; YP_003270181
atggaccacgctccggaacgctttgatgcgaccccgccggcaggtgaaccggaccgcccggcactgggtgtgctggaactgacctcaattgctcgtggtatcaccgttgcggatgcggccctgaaacgtgcaccgagtctgctgctgatgtcccgcccggtcagctctggcaagcatctgctgatgatgcgtggccaggtggcagaagttgaagaatcaatgattgcagctcgcgaaatcgctggtgcaggttcgggtgctctgctggatgaactggaactgccgtatgcgcacgaacaactgtggcgctttctggacgcaccggtggttgcagatgcatgggaagaagacaccgaaagcgtcattatcgtggaaaccgcgacggtgtgcgcggccattgatagtgccgacgcagctctgaaaacggcaccggtcgtgctgcgtgatatgcgcctggccattggtatcgctggcaaggcgtttttcaccctgacgggtgaactggcagacgtggaagcggccgcagaagttgtccgtgaacgttgcggtgcacgtctgctggaactggcatgtatcgcacgcccggttgatgaactgcgtggccgcctgtttttctaa

SEQ ID NO: 10 - protein, 206 aa, Haliangium ochraceum
MELRAYTVLDALQPQLVAFLQTVSTGFMPMEQQASVLVEIAPGIAVNQLTDAALKATRCQPGLQIVERAYGLIEMHDDDQGQVRAAGDAMLAHLGAREADRLAPRVVSSQIITGIDGHQSQLINRMRHGDMIQAGQTLYILEVHPAGYAALAANEAEKAAPIKLLEVVTFGAFGRLWLGGGEAEIAEAARAAEGALAGLSGRDNRG

SEQ ID NO: 11 - DNA, 621 bp, Artificial Sequence
  Synthetic DNA sequence selected to encode the protein sequence of SEQ ID NO10 while utilizing high-frequency codons from E. coli, the host organism; YP_003267736
atggaactgcgtgcttatacggtcctggatgccctgcagccgcaactggtcgcctttctgcaaacggtgtcaacgggtttcatgccgatggaacagcaagcgagcgttctggtcgaaattgcaccgggtatcgctgtcaaccagctgaccgacgcagcactgaaagcaacgcgttgccagccgggtctgcaaattgtggaacgtgcgtatggcctgatcgaaatgcatgatgacgatcagggtcaagttcgtgcagctggtgacgcaatgctggcacacctgggtgcacgtgaagctgatcgtctggcaccgcgtgtggttagctctcagattatcaccggtattgacggccatcagagtcaactgatcaaccgtatgcgccacggtgatatgattcaggcaggccaaacgctgtatatcctggaagttcatccggcaggttacgcagcactggcagctaatgaagccgaaaaagcggccccgattaagctgctggaagtcgtgacctttggtgcattcggtcgtctgtggctgggtggtggtgaagcagaaatcgcagaagcagctcgtgcggcagaaggtgcactggctggtctgtccggccgtgataatcgcggctaa

SEQ ID NO: 12 - protein, 212 aa, Haliangium ochraceum
MSITLRTYIFLDALQPQLATFIGKTARGFLPVPGQASLWVEIAPGIAINRVTDAALKATKVQPAVQVVERAYGLLEVHHFDQGEVLAAGSTILDKLEVREEGRLKPQVMTHQIIRAVEAYQTQIINRNSQGMMILPGESLFILETQPAGYAVLAANEAEKAANVHLVNVTPYGAFGRLYLAGSEAEIDAAAEAAEAAIRSVSGVAQESFRDR

SEQ ID NO: 13 - DNA, 639 bp, Artificial
  Synthetic DNA sequence selected to encode the protein sequence of SEQ ID NO12 while utilizing high-frequency codons from E. coli, the host organism; YP_003270185
atgtcaatcaccctgcgcacctatatctttctggacgccctgcaaccgcaactggcaaccttcatcggcaaaacggctcgtggcttcctgccggtcccgggtcaggcaagcctgtgggtggaaattgctccgggtattgcgatcaaccgtgtgaccgatgcggccctgaaagctacgaaggtgcagccggcggttcaagtggttgaacgcgcgtatggcctgctggaagttcatcacttcgatcagggcgaagtcctggcagctggtagtaccatcctggacaaactggaagttcgtgaagaaggtcgcctgaagccgcaggtgatgacccatcaaattatccgtgctgttgaagcgtatcagacgcaaattatcaaccgcaatagtcagggcatgatgattctgccgggtgaatccctgtttatcctggaaacccaaccggcaggttacgcagtcctggcagccaatgaagccgaaaaagcagctaacgttcacctggtcaatgtgacgccgtatggcgcattcggtcgtctgtacctggccggctcagaagcagaaattgatgcggccgcagaagctgcggaagccgcaatccgcagcgtttctggtgtcgcgcaggaatcgtttcgtgaccgctaa

SEQ ID NO: 14 - protein, 92 aa, Haliangium ochraceum
MYLGRVIGTVVAERKVAGLEGAKLLLVQPLDDALSPVGGVQAAVDTVQAGPDDLVYLVGSREAALALTPSFVPVDAAIVGIVDDVHAPERAS

SEQ ID NO: 15 - DNA, 279 bp, Artificial Sequence
  Synthetic DNA sequence selected to encode the protein sequence of SEQ ID NO14 while utilizing high-frequency codons from E. coli, the host organism; YP_003268812
atgtatctgggtcgtgtgattggtaccgtggtggctgaacgcaaagtggcgggtctggaaggcgcaaaactgctgctggtgcaaccgctggatgacgcactgagtccggtcggtggtgtgcaggcagcagttgataccgtccaagcaggtccggatgacctggtgtatctggttggtagccgtgaagcagctctggcgctgacgccgtcttttgtgccggttgatgcggccattgtcggcatcgttgatgacgtgcatgcaccggaacgcgctagctaa

SEQ ID NO: 16 - protein, 127 aa, Haliangium ochraceum
MRLCRVLGSVVATVKHPVYNGLPLMIVQPLDDAGRDAGASFLAVDNVQSGPGDRVLVLTEGGGVRQILALGDQVPIRSLIVGVVDAVDGVAATGVDDAGGAADSAAAAKSVRADELPADASAAGRGE

SEQ ID NO: 17 - DNA, 384 bp, Artificial Sequence
  Synthetic DNA sequence selected to encode the protein sequence of SEQ ID NO16 while utilizing high-frequency codons from E. coli, the host organism; YP_003268813
atgcgtctgtgtcgtgttctgggctccgtcgtcgccaccgtcaagcacccggtctacaatggtctgccgctgatgatcgttcaaccgctggatgacgcaggtcgtgatgcaggcgctagttttctggctgttgataacgtccagtccggtccgggtgaccgtgtcctggtgctgaccgaaggtggtggtgtgcgtcagattctggcactgggtgatcaagtcccgattcgcagcctgatcgtgggcgtggttgatgcagtggacggtgttgcagcaacgggtgttgatgacgcaggtggtgcagctgatagcgcagcagcagctaaatctgtccgtgcagatgaactgccggcagacgcaagcgcggccggtcgcggcgaataa

SEQ ID NO: 18 - protein, 96 aa, Haliangium ochraceum
MVLGKVVGTVVASRKEPRIEGLSLLLVRACDPDGTPTGGAVVCADAVGAGVGEVVLYASGSSARQTEVTNNRPVDATIMAIVDLVEMGGDVRFRKD

SEQ ID NO: 19 - DNA, 291 bp, Artificial Sequence
  Synthetic DNA sequence selected to encode the protein sequence of SEQ ID NO18 while utilizing high-frequency codons from E. coli, the host organism; YP_003270183
atggtcctgggtaaagtcgtgggtacggtggtggcgagccgcaaagaaccgcgcattgaaggtctgagcctgctgctggtccgtgcctgcgatccggacggtaccccgacgggtggtgcagtggtttgtgcagatgcagtgggtgcaggtgttggtgaagtcgtgctgtatgcgagtggcagctctgcccgtcagaccgaagtcacgaacaatcgcccggttgatgcaaccattatggctatcgttgacctggtcgaaatgggcggtgatgtgcgttttcgcaaagactaa

SEQ ID NO: 20 - DNA, 41 bp, Escherichia coli
tctagaaataattttgtttagagaaagaggagaaatactag

SEQ ID NO: 21 - DNA, 41 bp, Escherichia coli; identical to SEQ ID NO: 20

SEQ ID NO: 22 - DNA, 28 bp, Escherichia coli
tttagagattaaagaggagaaatactag

SEQ ID NO: 23 - protein, 55 aa, Artificial Sequence
  Part of Tag-GFP construct
MALREDRIAEIVERVLARLDGNSGSSAAPHSGSGAGSGSGSGSGSGSGSGSGSGS

SEQ ID NO: 24 - DNA, 165 bp, Artificial Sequence
  Part of Tag-GFP construct synthetic DNA sequence used with Tag-GFP construct of SEQ ID 23 derived from the N-terminus of an aldehyde dehydrogenase from H. ochraceum
atggcactgcgtgaagatcgtatcgctgaaatcgtggaacgtgtcctggcccgtctggatggcaactcgggctcgtcggctgcaccgcatagcggctctggtgcgggtagcggttcgggttcgggctctggttctggtagtggcagtggtagtggttcgggctcc

SEQ ID NO: 25 - protein, 505 aa, Artificial Sequence
  Part of Full Enzyme-GFP construct; aldehyde dehyrdogenase (Protein accession number  YP_003270182; Locus tag Hoch_5813)
MALREDRIAEIVERVLARLDGNSGSSAAPHSGSGAPAATAGGASLDIPRGTLGVYADADAAVNAARRGFAANEALPLRTRQAMIDAMRKVARAHIPELARYAVAETGLGRYEDKLAKNELVIAKTPGPEILAPVAYTGDDGLTLTERAPYGVIGAITPCTNPTETVICNAIGMLSGGNAVVFNVHPSAARVCNWLVHLLNEAIMSVGGPRDAITSVESPTIDSAQTLMTHAGVRLVVVTGGPGVVRAAMKSGKKVIAAGPGNPPAVVDETANLAKAAAAIIKGASIDNNIICTAEKEIVAVASIADELSRLLGQRGALVLGDAQVRALERVVLDGEHVNKEWVGKDASRIAEQIGLRGHGSDLRLLVCPVDEGHPFVQHELLMPVIGLVRVSDATEAMATAVRVEHGFCHTAVMHSTHIDRLSAMARVCNASIFVKNDCNLAGLGLGGEGFTSFTIASPTGEGLTTARDFTRVRRCTLKESFRFVGSGSGSGSGSGSGSGSGSGS

SEQ ID NO: 26 - DNA, 1515 bp, Artificial Sequence
  Synthetic DNA sequence to encode DNA sequence used to encode the fused protein of SEQ ID NO25 as Part of Full Enzyme-GFP construct
atggctctgcgtgaagatcgtatcgctgaaatcgtggaacgtgtcctggcccgtctggatggtaactccggctcgtcggctgccccgcacagcggctctggtgcgccggcggccaccgctggcggtgcgagcctggatattccgcgtggcacgctgggtgtgtatgcagatgctgacgcagctgttaacgcagcacgtcgcggttttgcagctaatgaagccctgccgctgcgtacccgtcaggcaatgattgatgcaatgcgtaaagttgcgcgcgcccatatcccggaactggcacgttatgcagtggctgaaaccggcctgggtcgctacgaagataaactggctaagaacgaactggttattgcgaagacgccgggcccggaaatcctggcaccggtggcatatacgggcgatgacggtctgaccctgacggaacgcgccccgtacggcgttattggtgcaatcaccccgtgcacgaacccgaccgaaacggtgatttgtaatgccatcggtatgctgtcaggcggtaacgcagtggttttcaatgtgcatccgtcggcggcccgtgtttgcaactggctggtccacctgctgaatgaagctattatgagtgttggcggtccgcgcgatgccattaccagtgtcgaatccccgacgatcgactccgctcaaaccctgatgacgcacgcgggcgttcgtctggtcgtggttaccggcggtccgggcgtcgtgcgtgcagctatgaaatcaggtaaaaaggttatcgcggcgggtccgggcaacccgccggcggttgtggatgaaaccgctaatctggcgaaagcagctgcggccattatcaagggtgcatcgatcgataacaatattatctgtaccgcggaaaaagaaattgtcgcggtggccagcatcgcagacgaactgtctcgtctgctgggtcagcgtggtgcgctggtcctgggcgatgctcaagtgcgtgcgctggaacgcgtggttctggacggcgaacatgtgaacaaagaatgggttggcaaggatgccagccgtattgcagaacagatcggtctgcgtggccacggttctgatctgcgtctgctggtctgcccggtggacgaaggccatccgtttgtccaacacgaactgctgatgccggtgattggtctggttcgtgtcagcgatgccaccgaagcaatggctacggcggtgcgcgttgaacatggcttttgtcacaccgcggtgatgcatagtacgcacattgaccgtctgtccgcgatggcgcgtgtgtgcaatgcgtctatcttcgtcaaaaacgattgtaatctggcaggtctgggtctgggcggtgaaggttttacctcattcacgatcgcatcgccgaccggtgaaggtctgaccacggcacgtgatttcacccgcgttcgtcgctgcacgctgaaagaatcttttcgcttcgtgggttctggtagtggctctggttcaggcagtggctcaggctcaggctcgggcagtggttct

SEQ ID NO: 27 - protein, 31 aa, Artificial Sequence
  Synthetic protein sequence fused to C-terminus of GFP of a Part of Noncognate Tag-GFP construct
GSGSGSGSGSEPEDNEDVQAIVKAIMAKLNL

SEQ ID NO: 28 - DNA, 99 bp, Artificial Sequence
  Synthetic DNA sequence fused to the 3′ end of the DNA encoding GFP of Part of Noncognate Tag-GFP construct
ctcgagggcagcggcagcggcagcggcagcggctctgaaccggaagacaatgaagatgtgcaggcaatcgtgaaagcaattatggctaaactgaacctg

SEQ ID NO: 29 - DNA, 30 bp, Artificial Sequence
  Synthetic RBS sequence for operon of Example 4 derived from E.coli
ttttgtttagagaaagaggagaaatactag

SEQ ID NO: 30 - protein, 93 aa, Mycobacterium smegmatis
MSSNAIGLIETKGYVAALAAADAMVKAANVTITDRQQVGDGLVAVIVTGEVGAVKAATEAGAETASQVGELVSVHVIPRPHSELGAHFSVSSK

SEQ ID NO: 31 - DNA, 282 bp, Mycobacterium smegmatis
atgagcagcaatgcaatcggtctgatcgaaacgaaaggctatgtggcggcactggcagcggcggatgcaatggtgaaggcagcaaatgtcaccattacggatcgtcagcaagttggcgacggtctggtggcggttatcgtcaccggcgaagtgggtgccgttaaagcggccaccgaagcaggcgctgaaacggcaagtcaagtgggtgaactggtgtccgttcatgtcattccgcgtccgcacagcgaactgggtgcacattttagcgttagctctaagtaa

SEQ ID NO: 32 - DNA, 28 bp, Artificial Sequence
  Synthetic RBS sequence for operon of Example 4 derived from E.coli
tttagagattaaagaggagaaatactag

SEQ ID NO: 33 - protein, 202 aa, Mycobacterium smegmatis
MAELRSFIFIDRLQPQTMSYLGTWIKGALPRANMAAQIIEVAPGLDIEGVTDVALKHAEVKAGILVVERQFGYLEFHGETGAVKAAADAALDYLGGDPDAAVRPEILASRIISSIDHQHAFLINRNKIGSMVLPGESLFVLEVAPASYAILATNEAEKAADVKVVDFRMIGATGRVYLSGTEADVRQAADAARDALAVLQGA

SEQ ID NO: 34 - DNA, 609 bp, Mycobacterium smegmatis
atggccgaactgcgtagcttcattttcattgaccgcctgcaaccgcaaacgatgtcctatctgggcacctggattaagggtgctctgccgcgtgcgaacatggcggcccagattatcgaagttgccccgggcctggatattgaaggtgttaccgacgtcgccctgaaacatgcagaagtcaaggctggcatcctggtggttgaacgccaatttggttatctggaatttcatggcgaaacgggtgcggtgaaagcagctgcggatgccgcactggactacctgggtggtgatccggacgctgcagttcgtccggaaattctggcctctcgcattatcagctctatcgatcatcagcacgcatttctgattaaccgtaataagatcggcagtatggtcctgccgggtgaatccctgttcgtgctggaagttgctccggcgagctatgcgattctggcgaccaatgaagcggaaaaagccgcagatgttaaggtcgtggactttcgtatgatcggtgcaaccggtcgtgtctacctgtcgggcacggaagctgatgtgcgtcaggctgcagatgcagcacgcgacgcactggcagtgctgcaaggtgcctaa

SEQ ID NO: 35 - DNA, 27 bp, Artificial Sequence
  Synthetic RBS sequence for operon of Example 4 derived from E.coli
tttagagtcacacaggaaacctactag

SEQ ID NO: 36 - protein, 87 aa, Mycobacterium smegmatis
MLRATVTGNVWSTRRIEGIPAGAFLEVEVEGTGSRMIAFDVLGSGVGEHVLIAQGSVASSWFTGTPPPIDALIIGSIDTRSDSNPAE

SEQ ID NO: 37 - DNA, 264 bp, Mycobacterium smegmatis
atgctgcgtgctaccgttaccggcaatgtctggtctacccgtcgtatcgaaggcatcccggctggtgcttttctggaagtggaagtcgaaggcaccggttcacgtatgattgcctttgatgtcctgggctcgggtgtgggcgaacatgttctgatcgcgcagggtagcgttgccagctcttggttcaccggtacgccgccgccgattgacgcactgattatcggtagtatcgatacgcgcagtgactccaacccggctgaataa

SEQ ID NO: 38 - protein, 471 aa, Artificial Sequence
  Synthetic non-native enzyme Rubisco protein sequence
MAAKKYSAGVKEYRQTYWTPDYVPLDTDLLACFKVTPQPGVPREEAAAAVAAESSTGTWTTVWTDLLTDMDYYKGRCYRIEDVPGDDESFYAFIAYPLDLFEEGSVTNVLTSLVGNVFGFKALRALRLEDIRFPMAYVKTCAGPPHGIQVERDKMNKYGRPLLGCTIKPKLGLSAKNYGRAVYECLRGGLDFTKDDENINSQPFQRWRDRFEFVAEAVEKAEAETGERKGHYLNVTAPTPEEMYKRAEFAKELGAPIIMHDYITGGFTANTGLAKWCRDNGVLLHIHRAMHAVIDRHPNHGIHFRVLAKCLRLSGGDHLHTGTVVGKLEGDRASTLGYIDLLRESFIPEDRSRGIFFDQDWGSMPGVFAVASGGIHVWHMPALVSIFGDDSVLQFGGGTLGHPWGNAAGAAANRVALEACVQARNEGRDIEKEGKDILTEAAKHSPELAIALETWKEIKFEFDTVDKLDTQ

SEQ ID NO: 39 - DNA, 1419 bp, Artificial Sequence
  Synthetic non-native enzyme Rubisco DNA sequence
catatggcagcgaaaaaatacagcgcaggcgtgaaagaataccgccaaacctactggactcccgattatgttcccctcgatacggacctcctggcctgctttaaagttaccccccagccaggtgtgccccgcgaagaggcagcagctgcagtcgcagcagaaagctcgactggcacctggaccacggtttggaccgacctgctcacggatatggactactataagggtcgctgttaccgcatcgaggatgtgcctggcgatgacgaaagcttttacgctttcattgcatatccattggatctgtttgaagagggctcggttactaacgtgctgaccagtctcgtcggtaatgtttttggcttcaaagccctgcgcgcgctccgcttggaagatatccgcttcccgatggcctacgtgaagacctgcgcaggtcccccgcatggcattcaagtcgaacgcgataaaatgaacaagtatggtcgccccttgctgggctgcacgatcaaaccgaagctgggtctctcggctaaaaattacggccgcgccgtgtatgaatgtttgcgcggcggtctggattttaccaaggatgacgagaacattaatagccagcccttccaacgctggcgcgatcgctttgaatttgtggcggaagctgtcgagaaagcagaagccgagacgggcgagcgcaagggccattacctgaacgtcaccgcgcctacgccagaagagatgtataaacgcgctgaatttgcaaaggagctcggcgctcccatcattatgcacgattacatcaccggcggtttcactgccaacaccggtttggcgaaatggtgccgcgacaatggcgttctcttgcacatccatcgcgccatgcacgcggtgattgatcgccacccgaatcatggcatccactttcgcgtcctcgcgaaatgtttgcgcctgagtggcggtgatcacttgcatacgggtactgtggtcggcaagttggaaggtgaccgcgccagcaccctgggctatattgatctgctccgcgagagctttatcccggaagatcgctcgcgcggcatctttttcgatcaggactggggctcgatgcccggtgtgttcgcagtcgctagtggtggtatccatgtgtggcacatgccggcgctcgtcagtatttttggcgatgacagcgtgctgcagttcggtggtggtaccctcggtcatccttggggtaacgctgcaggtgcagcagctaatcgcgtcgctctggaggcatgcgttcaagcccgcaacgaaggtcgcgacatcgaaaaagagggcaaggatattctcactgaggcagccaagcacagcccggaactcgcaatcgccttggaaacgtggaaagagattaagtttgaatttgatacggtcgacaaactggatactcaatag

SEQ ID NO: 40 - protein, 102 aa, Thermosynechococcus elongatus
MPIAVGMIETRGFPAVVEAADAMVKAARVTLVGYEKIGSGRVTVIVRGDVSEVQASVAAGVDSAKRVNGGEVLSTHIIARPHENLEYVLPIRYTEAVEQFRN

SEQ ID NO: 41 - DNA, 309 bp, Artificial sequence
  Synthetic hexamer DNA sequence for synthetic operon of Example 6 from Thermosynechococcus elongatus BP-1 NC_004113. This sequence has been codon-optimized for expression in E. coli
atgccaattgctgtgggaatgattgaaacgcgcggatttcccgccgtcgtcgaagcagcagatgcaatggtcaaagccgctcgggttaccctggtgggctacgaaaaaattgggagtgggcgggtcaccgtgattgtgcggggtgatgtctccgaagtgcaagcgtcagtagctgccggggtcgattctgccaagcgtgtcaatggcggagaggtgctgtccacgcacattattgcccgtccccacgaaaaccttgagtacgtattgcccattcgctataccgaggcagtggagcaattccgaaactaa

SEQ ID NO: 42 - protein, 253 aa, Thermosynechococcus elongatus
MERRDDFTDLALGLVSVQSFPAIVGIADHMLKSSDVLLVGYEKIGGGHCTAIVRGRIADVRLAVEEGAERAQQFGQELSTLVIPRPDPNLEKILPIGSLLAQIASKSRGHRLSSHAVGLLETRGFPAMVGAADAMLKAADVMLTAYETIGAGLCTAIIRGTASNTAIALEAGMAEADRIGELHAVMLVPRPLEDLDQSLPLAPALQRELQPLRLPLTLKQKETEPLALQGAAQASVAVEAAAERVPVDPPANP

SEQ ID NO: 43 - DNA, 762 bp, Artificial sequence
  Synthetic tandem domain DNA sequence for synthetic operon of Example 6 from Thermosynechococcus elongatus BP-1 NC_004113. This sequence has been codon-optimized for expression in E. coli
atggagcgacgggatgactttacggatttagccttagggctggtctcagtccagagctttccggcgatcgttggcattgccgatcacatgctcaaatcctccgatgtcctcctagtgggctatgaaaaaattggcggtggtcactgtaccgcgatcgtccgcgggcgaattgccgatgtgcgccttgcggtagaagagggggccgagcgggcgcagcaattcggtcaggaactgagtacgttagtgattccccgacccgatcccaacctagagaaaattctccccattggcagtctccttgcccagattgcttctaaaagtcgcggccatcgcctcagtagccatgccgtaggtcttctggaaacccggggatttccagccatggtgggggcagccgatgccatgctcaaggcagcggatgtgatgctgacggcctacgaaaccattggggcagggttatgtacggctattattcgcggtacggcctccaataccgcgatcgccctcgaggccggaatggcagaagccgatcgcattggtgaactccatgcggtgatgttggtgccccgtccccttgaggatttggatcaatccttgcccttggcacctgccctccaacgggaactgcaacccctgcgtctcccccttaccctcaagcaaaaagaaaccgaaccccttgccctccaaggggcggctcaagcgagtgtggctgtggaagccgccgccgaaagggtgcccgtcgacccccctgccaatccctga

SEQ ID NO: 44 - protein, 99 aa, Thermosynechococcus elongatus
MKIARVCGTVTSTQKEDTLTGVKFLVLQYLGEDGEFLPDYEVAADTVGAGQDEWVLVSRGSAARHIINGTDKPIDAAVVAIIDTVSRDNYLLYSKRTQY

SEQ ID NO: 45 - DNA, 300 bp, Artificial sequence
  Synthetic pentamer DNA sequence for synthetic operon of Example 6 from Thermosynechococcus elongatus BP-1 NC_004113. This sequence has been codon-optimized for expression in E. coli
gtgaaaatcgcgcgagtgtgcggcaccgttaccagtactcaaaaagaagacaccttaacgggagtcaagtttctcgtcttgcaatatttgggtgaggacggcgaatttttacccgactacgaagtggctgcggatacggttggtgcaggacaggatgagtgggtattggtaagccgaggcagtgccgcccgccatattatcaatggcaccgacaaacccattgacgcagccgttgtcgccattattgacaccgtaagtcgggataattatttgctctacagcaaacgtacccagtattag

SEQ ID NO: 46 - DNA, 19 bp, Artificial sequence
  Synthetic B1010 ribosome binding site
tttaagaaggagatatacc

SEQ ID NO: 47 - DNA, 20 bp, Artificial sequence
  Synthetic B1001 ribosome binding site
ggctaacatagggtggatct

SEQ ID NO: 48 - protein, 238 aa, artificial
  Synthetic Protein sequence of SuperFolderGFP used in the SFGFP-CcmK2 fusion proteins
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 49 - DNA, 714 bp, Artificial sequence
  Synthetic construct sfGFP_iGEM gene, complete cds [synthetic contstruct, GI532528632]
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaa

SEQ ID NO: 50 - protein, 55 aa, Haliangium ochraceum
AGLGQAADAATQHAHAYAAAPRQSASESASGGGRDDLVRVIREELVRALAGEESR

SEQ ID NO: 51 - protein, 16 aa, Haliangium ochraceum
RDDLVRVIREELVRAL

SEQ ID NO: 52 - DNA, 165 bp, Haliangium ochraceum
gcgggtctgggtcaggcagcagacgcagctacccaacatgctcacgcatacgcagcagcaccgcgtcagtcagcttcggaaagcgcatctggcggtggccgtgatgacctggtccgtgtgatccgcgaagaactggtgcgtgccctggcaggtgaagaatcccgc

SEQ ID NO: 53 - protein, 18 aa, Haliangium ochraceum
ALREDRIAEIVERVLARL

SEQ ID NO: 54 - DNA, 54 bp, Haliangium ochraceum
gcgctgcgcgaagatcgcattgcggaaattgtggaacgcgtgctggcgcgcctg

SEQ ID NO: 55 - DNA, 28 bp, Artificial sequence
  Synthetic Ribosomal binding site for BMC-H
tctagagaaagaggagaaatactagatg

SEQ ID NO: 56 - DNA, 31 bp, Artificial
  Synthetic Ribosomal binding site for BMC-T
tctagagattaaagaggagaaatactagatg

SEQ ID NO: 57 - DNA, 30 bp, Artificial
  Synthetic Ribosomal binding site for BMC-P
tctagagtcacacaggaaacctactagatg

SEQ ID NO: 58 - DNA, 3332 bp, Artificial
  Full sequence of the synthetic H. ochraceum operon
aataattttgtttagagaaagaggagaaatactagatggcggacgcactgggtatgattgaagttcgtggttttgttggtatggtggaagcggcggatgctatggtgaaagcggctaaagttgaactgattggttatgaaaaaaccggcggtggctacgtgacggcagtggttcgtggtgatgtcgcagcagttaaggcagctaccgaagccggtcagcgtgcagcagaacgtgttggtgaagtcgtggcagttcatgtcatcccgcgtccgcacgtgaacgttgatgcagctctgccgctgggtcgtacgccgggtatggacaaaagcgcgtaatttagagattaaagaggagaaatactagatggaccacgctccggaacgctttgatgcgaccccgccggcaggtgaaccggaccgcccggcactgggtgtgctggaactgacctcaattgctcgtggtatcaccgttgcggatgcggccctgaaacgtgcaccgagtctgctgctgatgtcccgcccggtcagctctggcaagcatctgctgatgatgcgtggccaggtggcagaagttgaagaatcaatgattgcagctcgcgaaatcgctggtgcaggttcgggtgctctgctggatgaactggaactgccgtatgcgcacgaacaactgtggcgctttctggacgcaccggtggttgcagatgcatgggaagaagacaccgaaagcgtcattatcgtggaaaccgcgacggtgtgcgcggccattgatagtgccgacgcagctctgaaaacggcaccggtcgtgctgcgtgatatgcgcctggccattggtatcgctggcaaggcgtttttcaccctgacgggtgaactggcagacgtggaagcggccgcagaagttgtccgtgaacgttgcggtgcacgtctgctggaactggcatgtatcgcacgcccggttgatgaactgcgtggccgcctgtttttctaatttagagattaaagaggagaaatactagatggaactgcgtgcttatacggtcctggatgccctgcagccgcaactggtcgcctttctgcaaacggtgtcaacgggtttcatgccgatggaacagcaagcgagcgttctggtcgaaattgcaccgggtatcgctgtcaaccagctgaccgacgcagcactgaaagcaacgcgttgccagccgggtctgcaaattgtggaacgtgcgtatggcctgatcgaaatgcatgatgacgatcagggtcaagttcgtgcagctggtgacgcaatgctggcacacctgggtgcacgtgaagctgatcgtctggcaccgcgtgtggttagctctcagattatcaccggtattgacggccatcagagtcaactgatcaaccgtatgcgccacggtgatatgattcaggcaggccaaacgctgtatatcctggaagttcatccggcaggttacgcagcactggcagctaatgaagccgaaaaagcggccccgattaagctgctggaagtcgtgacctttggtgcattcggtcgtctgtggctgggtggtggtgaagcagaaatcgcagaagcagctcgtgcggcagaaggtgcactggctggtctgtccggccgtgataatcgcggctaatttagagattaaagaggagaaatactagatgtcaatcaccctgcgcacctatatctttctggacgccctgcaaccgcaactggcaaccttcatcggcaaaacggctcgtggcttcctgccggtcccgggtcaggcaagcctgtgggtggaaattgctccgggtattgcgatcaaccgtgtgaccgatgcggccctgaaagctacgaaggtgcagccggcggttcaagtggttgaacgcgcgtatggcctgctggaagttcatcacttcgatcagggcgaagtcctggcagctggtagtaccatcctggacaaactggaagttcgtgaagaaggtcgcctgaagccgcaggtgatgacccatcaaattatccgtgctgttgaagcgtatcagacgcaaattatcaaccgcaatagtcagggcatgatgattctgccgggtgaatccctgtttatcctggaaacccaaccggcaggttacgcagtcctggcagccaatgaagccgaaaaagcagctaacgttcacctggtcaatgtgacgccgtatggcgcattcggtcgtctgtacctggccggctcagaagcagaaattgatgcggccgcagaagctgcggaagccgcaatccgcagcgtttctggtgtcgcgcaggaatcgtttcgtgaccgctaatttagagtcacacaggaaacctactagatgtatctgggtcgtgtgattggtaccgtggtggctgaacgcaaagtggcgggtctggaaggcgcaaaactgctgctggtgcaaccgctggatgacgcactgagtccggtcggtggtgtgcaggcagcagttgataccgtccaagcaggtccggatgacctggtgtatctggttggtagccgtgaagcagctctggcgctgacgccgtcttttgtgccggttgatgcggccattgtcggcatcgttgatgacgtgcatgcaccggaacgcgctagctaatttagagtcacacaggaaacctactagatgcgtctgtgtcgtgttctgggctccgtcgtcgccaccgtcaagcacccggtctacaatggtctgccgctgatgatcgttcaaccgctggatgacgcaggtcgtgatgcaggcgctagttttctggctgttgataacgtccagtccggtccgggtgaccgtgtcctggtgctgaccgaaggtggtggtgtgcgtcagattctggcactgggtgatcaagtcccgattcgcagcctgatcgtgggcgtggttgatgcagtggacggtgttgcagcaacgggtgttgatgacgcaggtggtgcagctgatagcgcagcagcagctaaatctgtccgtgcagatgaactgccggcagacgcaagcgcggccggtcgcggcgaataatttagagtcacacaggaaacctactagatggtcctgggtaaagtcgtgggtacggtggtggcgagccgcaaagaaccgcgcattgaaggtctgagcctgctgctggtccgtgcctgcgatccggacggtaccccgacgggtggtgcagtggtttgtgcagatgcagtgggtgcaggtgttggtgaagtcgtgctgtatgcgagtggcagctctgcccgtcagaccgaagtcacgaacaatcgcccggttgatgcaaccattatggctatcgttgacctggtcgaaatgggcggtgatgtgcgttttcgcaaagactaa